(19) United States
(12) Patent Application Publication
(10) Pub. No.: US 2024/0216283 A1
(43) Pub. Date: Jul. 4, 2024

(54) GAMMA-HYDROXYBUTYRATE FORMULATIONS HAVING IMPROVED STABILITY AFTER STORAGE

(71) Applicant: Flamel Ireland Limited, Dublin (IE)

(72) Inventor: Hervé Guillard, Villeurbanne (FR)

(21) Appl. No.: 18/605,502

(22) Filed: Mar. 14, 2024

Related U.S. Application Data
(63) Continuation-in-part of application No. 17/156,053, filed on Jan. 22, 2021, now abandoned.
Application No. 17/156,053 is a continuation of application No. 16/223,940, filed on Dec. 18, 2018, now abandoned.
(60) Provisional application No. 62/607,937, filed on Dec. 20, 2017.
(60) Provisional application No. 62/618,832, filed on Jan. 18, 2018.

(51) Int. Cl. A61K 9/50; A61J 1/10; A61J 1/14; A61K 31/19; A61K 47/02; A61K 47/12; A61K 47/38; A61P 25/00

(52) U.S. Cl. CPC A61K 9/5026; A61J 1/1468; A61K 9/5015; A61K 9/5036; A61K 9/5047; A61K 9/5073; A61K 31/19; A61K 47/02; A61K 47/12; A61K 47/38; A61P 25/00; A61J 1/10

(57) ABSTRACT

Packaged formulations of gamma-hydroxybutyrate having improved dissolution and chemical stability after storage for up to at least 72 months, packaging for supporting said stability, and therapeutic uses thereof.

### CROSS-REFERENCE TO RELATED APPLICATIONS

This application is a continuation-in-part of U.S. application Ser. No. 17/156,053, filed Jan. 22, 2021, which is a continuation of U.S. application Ser. No. 16/223,940, filed Dec. 18, 2018, which claims priority to U.S. Provisional Application Ser. No. 62/607,937, filed Dec. 20, 2017, and U.S. Provisional Application Ser. No. 62/618,832, filed Jan. 18, 2018.

### FIELD OF THE INVENTION

The present invention relates to packaged modified release formulations of gamma-hydroxybutyrate having improved dissolution and chemical stability, packaging for supporting said stability, and to therapeutic uses thereof.

### BACKGROUND

Narcolepsy is a devastating disabling condition. The cardinal symptoms are excessive daytime sleepiness (EDS), cataplexy (a sudden loss of muscle tone triggered by strong emotions, seen in approximately 60% of patients), hypnogogic hallucination (HH), sleep paralysis (SP), and disturbed nocturnal sleep (DNS). Other than EDS, DNS is the most common symptom seen among narcolepsy patients.

One of the major treatments for narcolepsy is gamma-hydroxybutyrate, also known in its sodium form as sodium 4-hydroxybutanoate, sodium oxybate, gamma-hydroxybutyric acid sodium salt, or NaGHB. Gamma-hydroxybutyrate or GHB is a neuroactive agent with a variety of central nervous system (CNS) pharmacological properties. The species is present endogenously in many tissues, where it acts as a neurotransmitter on a gamma-hydroxybutyrate (GHB) receptor (GHBR), and possesses neuromodulatory properties with significant effects on dopamine and gamma-aminobutyric acid (GABA).

Gamma-hydroxybutyrate is marketed commercially in the United States as XYREM®. This product is formulated as an immediate release liquid solution that is taken once immediately before bed, and a second time approximately 2.5 to 4 hours later, in equal doses. For each dose, a measured amount of the oral solution must be removed from the primary container and transferred to a separate container where it is diluted with water before administration. The second dose is prepared at bedtime and stored for administration in the middle of the night. Sleep-onset can be dramatic and fast, and patients are advised to be sitting in bed when consuming the dose.

When initiating treatment with gamma-hydroxybutyrate, careful titration up to an adequate level is essential both to obtain positive results and avoid adverse effects. The recommended starting dose is 4.5 g divided into 2 equal doses of 2.25 g, the first taken at bedtime and the second taken 2.5 to 4 hours later. The starting dosage can be decreased to 3.0 g/day or increased to as high as 9.0 g/day in increments of 1.5 g/day (0.75 g per dose). Two weeks are recommended between dosage adjustments to optimize reduction of daytime symptoms and minimize side effects. The ideal dose will provide an effective eight hours of sleep but, at the end of eight hours, very little of the drug will remain in the patient's bloodstream to affect the patient's wakefulness.

The requirement to take XYREM® twice each night is a substantial inconvenience to narcolepsy patients. The patient must typically set an alarm to take the second dose, which can interrupt ongoing productive sleep. This regimen is cumbersome and prone to errors in the preparation of the individual doses. For these reasons, a more convenient unit dosage form of the drug would be clinically advantageous.

Several efforts have been made to provide a once-nightly modified release dosage form of gamma-hydroxybutyrate, but none has yet received approval from the United States Food and Drug Administration (“FDA”) or proven effective in the clinic. One of the biggest drawbacks of these once-nightly formulations is the reduction in bioavailability that occurs when gamma-hydroxybutyrate is formulated in a modified release dosage form, as measured by the blood concentration/time area under the curve (“AUC”). U.S. Patent Publication 2012/0076865 and U.S. Pat. No. 8,193,211 report relative bioavailabilities from their once-nightly formulations at a fraction of the immediate release dose. Due to the high amount of daily dose, up to 9 g per day, there is a need for once a day formulation of gamma-hydroxybutyrate that can provide a comparable bioavailability to current treatments so that the total daily dose need not be increased.

Formulating modified release solid dosage forms of gamma-hydroxybutyrate is challenging, not only because of the large amount of the drug that may be needed to achieve an adequate clinical response, but also because gamma-hydroxybutyrate is highly water-soluble, hygroscopic, and strongly alkaline. Gamma-hydroxybutyrate is prone to attract water from the environment, which in turns promotes a high local pH, migration of gamma-hydroxybutyrate, and interactions with excipients, which can promote the formation of GBL (gamma-butyrolactone) as a degradant or induce dissolution instability. These properties make it difficult to formulate a product that remains stable over time, particularly in terms of dissolution during storage and/or chemical stability. Thus, there is a need for solid modified release formulations of gamma-hydroxybutyrate that retain their dissolution profile over time, i.e. they have stable dissolution profiles, and are chemically stable over time, with reduced chemical degradation products.

### SUMMARY OF INVENTION

Among the various aspects of the present invention is the provision of packaged modified release formulations of gamma-hydroxybutyrate that have stable dissolution profiles over time. The packaged gamma-hydroxybutyrate compositions disclosed herein maintain chemical and dissolution stability, particularly when maintained within a defined range of relative humidity values.

The present invention, therefore, provides a packaged pharmaceutical composition comprising a modified release gamma-hydroxybutyrate pharmaceutical composition within a package. The pharmaceutical composition comprises (a) an immediate release component comprising gamma-hydroxybutyrate or a pharmaceutically acceptable salt thereof; and (b) a modified release component comprising gamma-hydroxybutyrate or a pharmaceutically acceptable salt thereof, wherein the package has an interior volume having a relative humidity from 29% to 54%, and the pharmaceutical composition has a stable dissolution profile over time.

In some aspects, the relative humidity of the package is from 29% to 54% for a period of at least 2 months when stored at 40° C. and 75% relative humidity. In other aspects, the relative humidity of the package is greater than 29% at 1 week and less than 54% at 2 months when stored at 40° C. and 75% relative humidity. In still other aspects, the relative humidity of the package is greater than 29% and less than 44% at one week and less than 54% at 2 months when stored at 40° C. and 75% relative humidity. In further aspects, the relative humidity of the package is from 35% to 39% after one week and from 39% to 48% after 2 months when stored at 40° C. and 75% relative humidity.

In other iterations, the packaging prevents no more than 0.4% of gamma-hydroxybutyrate in the pharmaceutical composition from being converted to gamma-butyrolactone (GBL) when stored two months at 40° C. and 75% relative humidity. In additional aspects, the package has a water vapor transmission rate of less than 7 mg/day/liter when measured according to USP 38<671>.

In further aspects, the pharmaceutical composition has a dissolution of gamma-hydroxybutyrate that differs by less than 10% than the dissolution of gamma-hydroxybutyrate before the storage period when tested for at least four consecutive hourly time points in a dissolution apparatus 2 according to USP 38<711> in 900 mL of 0.1N hydrochloric acid at a temperature of 37° C. and a paddle speed of 75 rpm when the packaged composition is stored for two months at 40° C. and 75% relative humidity. In still other iterations, the pharmaceutical composition has a dissolution of gamma-hydroxybutyrate that differs by less than 10% than the dissolution of gamma-hydroxybutyrate before the storage period when tested for at least four consecutive hourly time points in a dissolution apparatus 2 according to USP 38<711> in 900 mL of 0.1N hydrochloric acid at a temperature of 37° C. and a paddle speed of 75 rpm when the packaged composition is stored for two months at 40° C. and 75% relative humidity.

In additional aspects, the modified release component comprises a core comprising gamma-hydroxybutyrate or a pharmaceutically acceptable salt thereof and a coating comprising a hydrophobic compound and a mixture of methacrylic acid copolymers. In some aspects, the hydrophobic compound is glyceryl tristearate or hydrogenated vegetable oil, and the mixture of methacrylic acid copolymers comprises methacrylic acid and ethyl acrylate copolymer NF and methacrylic acid and methyl methacrylate copolymer (1:2) NF. In other aspects, the coating comprises from 40 to 70 weight parts of the hydrophobic compound and from 30 to 60 weight parts of the mixture of methacrylic acid copolymers, and the coating is from 10% to 50% of the weight of the modified release component. In further aspects, the hydrophobic compound has a melting point equal to or greater than 40° C. and the mixture of methacrylic acid copolymers has a pH trigger greater than 5.6. In some embodiments, the immediate release component comprises particles having an average diameter from 95 to 600 micrometers and/or the modified release component comprises particles having an average diameter from 200 to 800 micrometers.

In some aspects, the weight ratio of gamma-hydroxybutyrate in the immediate release and modified release components is from 10/90 to 65/35, or from 40/60 to 60/40. In certain aspects, the package comprises from 0.5 gram to 12.0 grams of sodium salt of gamma-hydroxybutyrate, e.g., 3.0 4.5, 6.0, 7.5 or 9.0 g of sodium oxybate. In specific aspects, the package is a pouch or sachet, e.g., an aluminum foil pouch or sachet having an aluminum foil thickness of at least 6 micrometers.

Another aspect of the present invention includes a pharmaceutical formulation stored within a package, the pharmaceutical formulation comprising: an immediate release portion comprising gamma-hydroxybutyrate or a pharmaceutically acceptable salt thereof; and a modified release portion comprising gamma-hydroxybutyrate or a pharmaceutically acceptable salt thereof, wherein the pharmaceutical formulation has a pH of less than 5.9 after the pharmaceutical formulation has been stored for up to at least 72 months at 25° C. to 40° C. and 60% to 75% relative humidity. In various aspects, the pharmaceutical formulation has a pH of 5.6 to 5.9 after the pharmaceutical formulation has been stored for up to 72 months, up to 60 months, up to 48 months, up to 36 months, up to 24 months, up to 18 months, up to 12 months, up to 6 months, or up to 3 months at 25° C. to 40° C. and 60% to 75% relative humidity.

Any of the pharmaceutical formulations disclosed herein may further include: one or more suspending or viscosifying agents selected from the group consisting of xanthan gum, medium viscosity sodium carboxymethyl cellulose, mixtures of microcrystalline cellulose and sodium carboxymethyl cellulose, mixtures of microcrystalline cellulose and guar gum, medium viscosity hydroxyethyl cellulose, agar, sodium alginate, mixtures of sodium alginate and calcium alginate, gellan gum, carrageenan gum, medium viscosity hydroxypropylmethyl cellulose, and mixtures thereof; and an acidifying agent selected from the group consisting of malic acid, citric acid, tartaric acid, adipic acid, boric acid, maleic acid, phosphoric acid, ascorbic acid, oleic acid, capric acid, caprylic acid, and benzoic acid, wherein the suspending or viscosifying agent and the acidifying agent are separate and distinct from the immediate release portion and the modified release portion. The suspending or viscosifying agent may be present at 1% to 15% by weight of the formulation, and the acidifying agent is present at 1.2% to 15% by weight of the formulation. The amount of gamma-hydroxybutyrate may be equivalent to 4.5 g, 6.0 g, 7.5 g, or 9.0 g of sodium oxybate.

In other aspects, the pharmaceutical formulation stored within a package may include: an immediate release portion comprising gamma-hydroxybutyrate or a pharmaceutically acceptable salt thereof; and a modified release portion comprising gamma-hydroxybutyrate or a pharmaceutically acceptable salt thereof, wherein less than 0.18% w/w gamma-butyrolactone is present in the pharmaceutical formulation after the pharmaceutical formulation has been stored for up to at least 72 months at 25° C. to 40° C. and 60% to 75% relative humidity. For example, less than 0.15% w/w gamma-butyrolactone is present in the pharmaceutical formulation after the pharmaceutical formulation has been stored for up to 72 months, up to 60 months, up to 48 months, up to 36 months, up to 24 months, up to 18 months, up to 12 months, up to 6 months, or up to 3 months at 25° C. to 40° C. and 60% to 75% relative humidity.

In additional aspects, the pharmaceutical formulation stored within a package may include: an immediate release portion comprising gamma-hydroxybutyrate or a pharmaceutically acceptable salt thereof; and a modified release portion comprising gamma-hydroxybutyrate or a pharmaceutically acceptable salt thereof, wherein the amount of gamma-hydroxybutyrate in the pharmaceutical formulation after the pharmaceutical formulation has been stored for up to at least 72 months at 25° C. to 40° C. and 60% to 75% relative humidity is 90.0% to 110.0% of the amount of gamma-hydroxybutyrate in the pharmaceutical formulation before storage. In some examples, the amount of gamma-hydroxybutyrate in the pharmaceutical formulation after the pharmaceutical formulation has been stored for up to 72 months, up to 60 months, up to 48 months, up to 36 months, up to 24 months, up to 18 months, up to 12 months, up to 6 months, or up to 3 months at 25° C. to 40° C. and 60% to 75% relative humidity is 99.0% to 105.0% of the amount of gamma-hydroxybutyrate in the pharmaceutical formulation before storage.

In further aspects, the pharmaceutical formulation stored within a package may include: an immediate release portion comprising gamma-hydroxybutyrate or a pharmaceutically acceptable salt thereof; and a modified release portion comprising gamma-hydroxybutyrate or a pharmaceutically acceptable salt thereof, wherein the water content in the pharmaceutical formulation is ≤4.0% w/w after the pharmaceutical formulation has been stored for up to at least 72 months at 25° C. to 40° C. and 60% to 75% relative humidity. For example, the water content in the pharmaceutical formulation is ≤1.0% w/w after the pharmaceutical formulation has been stored for up to 72 months, up to 60 months, up to 48 months, up to 36 months, up to 24 months, up to 18 months, up to 12 months, up to 6 months, or up to 3 months at 25° C. to 40° C. and 60% to 75% relative humidity.

In some aspects, the pharmaceutical formulation stored within a package may include: an immediate release portion comprising gamma-hydroxybutyrate or a pharmaceutically acceptable salt thereof; and a modified release portion comprising gamma-hydroxybutyrate or a pharmaceutically acceptable salt thereof, wherein after storage for up to at least 72 months at 25° C./60% RH, the pharmaceutical formulation has a dissolution of gamma-hydroxybutyrate that differs by less than 10% than the dissolution of gamma-hydroxybutyrate before the storage period when tested for at least three time points using a 0.1 N HCl dissolution test by HPLC or when tested after one hour using a pH 6.8 dissolution test by HPLC. For example, after storage for up to 72 months, up to 60 months, up to 48 months, up to 36 months, up to 24 months, up to 18 months, up to 12 months, up to 6 months, or up to 3 months at 25° C./60% RH, 30° C./65% RH, or 40° C./75% RH, the pharmaceutical formulation has a dissolution of gamma-hydroxybutyrate that differs by less than 10% than the dissolution of gamma-hydroxybutyrate before the storage period when tested for at least three time points using a 0.1 N HCl dissolution test by HPLC or when tested after one hour using a pH 6.8 dissolution test by HPLC.

In additional aspects, the pharmaceutical formulation stored within a package may include: an immediate release portion comprising gamma-hydroxybutyrate or a pharmaceutically acceptable salt thereof; and a modified release portion comprising gamma-hydroxybutyrate or a pharmaceutically acceptable salt thereof, wherein after storage for up to at least 72 months at 25° C./60% RH, 30° C./65% RH, or 40° C./75% RH, the pharmaceutical formulation has a dissolution of gamma-hydroxybutyrate of between about 40% and about 60% after 1 hour of dissolution in 0.1 N HCl, between about 45% and about 65% after 4 hours of dissolution in 0.1 N HCl, and greater than or equal to 80% dissolution after 12 hours of dissolution in 0.1 N HCl. In some examples, after storage for up to 72 months, up to 60 months, up to 48 months, up to 36 months, up to 24 months, up to 18 months, up to 12 months, up to 6 months, or up to 3 months at 25° C./60% RH, the pharmaceutical formulation has a dissolution of gamma-hydroxybutyrate of between about 40% and about 60% after 1 hour of dissolution in 0.1 N HCl, between about 45% and about 65% after 4 hours of dissolution in 0.1 N HCl, and greater than or equal to 80% dissolution after 12 hours of dissolution in 0.1 N HCl. In additional examples, after storage for up to 24 months, up to 18 months, up to 15 months, up to 12 months, up to 6 months, or up to 3 months at 30° C./65% RH, the pharmaceutical formulation has a dissolution of gamma-hydroxybutyrate of between about 40% and about 60% after 1 hour of dissolution in 0.1 N HCl, between about 45% and about 65% after 4 hours of dissolution in 0.1 N HCl, and greater than or equal to 80% dissolution after 12 hours of dissolution in 0.1 N HCl. In other examples, after storage for up to 6 months or up to 3 months at 40° C./75% RH, the pharmaceutical formulation has a dissolution of gamma-hydroxybutyrate of between about 40% and about 60% after 1 hour of dissolution in 0.1 N HCl, between about 45% and about 65% after 4 hours of dissolution in 0.1 N HCl, and greater than or equal to 80% dissolution after 12 hours of dissolution in 0.1 N HCl.

In additional aspects, A pharmaceutical formulation stored within a package, the pharmaceutical formulation comprising: an immediate release portion comprising gamma-hydroxybutyrate or a pharmaceutically acceptable salt thereof; and a modified release portion comprising gamma-hydroxybutyrate or a pharmaceutically acceptable salt thereof, wherein after storage for up to at least 72 months, up to 60 months, up to 48 months, up to 36 months, up to 24 months, up to 18 months, up to 12 months, up to 6 months, or up to 3 months at 25° C./60% RH, 30° C./65% RH, or 40° C./75% RH, the pharmaceutical formulation has a dissolution of gamma-hydroxybutyrate of >85% after 1 hour in a pH 6.8 dissolution test by HPLC. In some examples, after storage for up to 24 months, up to 18 months, up to 15 months, up to 12 months, up to 6 months, or up to 3 months at 30° C./65% RH, the pharmaceutical formulation has a dissolution of gamma-hydroxybutyrate of >85% after 1 hour in a pH 6.8 dissolution test by HPLC. In additional examples, after storage for up to 6 months or up to 3 months at 40° C./75% RH, the pharmaceutical formulation has a dissolution of gamma-hydroxybutyrate of ≥85% after 1 hour in a pH 6.8 dissolution test by HPLC.

Additional embodiments and sub-embodiments will be set forth in part in the description which follows, and in part will be obvious from the description, or may be learned by practice of the invention. The embodiments and advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE FIGURES

The patent or application file contains at least one drawing executed in color. Copies of this patent or patent application publication with color drawing(s) will be provided by the Office upon request and payment of the necessary fee.

FIG. 1 depicts the qualitative and quantitative structure of the immediate release (IR) and modified release (MR) microparticles of gamma-hydroxybutyrate of Example 1 (first formulation).

FIG. 2 plots the dissolution profile of a packaged formulation prior to and after storage for one month at 40° C. and 75% relative humidity, illustrating the computation of lag time according to the current invention, as described in Example 2.

FIG. 3 depicts the dissolution profile of a packaged formulation over six months at 40° C./75% RH in a PET/ALU/PE aluminum pouch (9 μm aluminum foil) from Bischof & Klein (Lengerich Germany), as described in Example 3.

FIG. 4 depicts the dissolution profile of a packaged formulation over six months at 40° C./75% RH in a CONSTANTIA™ stick-pack (PET/adhesive layer/ALU/copolymer with 12 μm aluminum foil), as described in Example 3.

FIG. 5 depicts the dissolution profile of a packaged formulation over six months at 40° C./75% RH in a LOG™ H2OO2 bottle, as described in Example 3.

FIG. 6 depicts the dissolution profile of a packaged formulation over five months at 40° C./75% RH in a LOG™ H2OO2 bottle, as described in Example 4

FIG. 7 depicts the dissolution profile of a packaged formulation over three months at 40° C./75% RH in a DUMA™ bottle (30 ml) without desiccant, as described in Example 4.

FIG. 8 depicts the dissolution profile of a packaged formulation over one month at 40° C./75% RH in a DUMA™ bottle (30 ml) with 2 g of silica gel desiccant, as described in Example 4.

FIG. 9 depicts the dissolution profile of a packaged formulation over one month at 40° C./75% RH in a DUMA™ bottle (30 ml) with 2 g of Intelisorb™ desiccant, as described in Example 4.

FIG. 10 plots the relative humidity over time inside sachets with aluminum foil, Duma bottles, Duma bottles with silica gel desiccant, Duma bottles with INTELLISORB™ desiccants, and LOG™ H2OO2 bottles, when maintained in a climatic chamber at 40° C. and 75% RH, as described in Example 4.

FIG. 11 is FIG. 10 with dots overlaid to indicate RH values at which the packaged formulation was considered stable (clear circles), unstable due to a slowdown of the dissolution profile (hatched circles), or unstable due to an acceleration of the dissolution profile (black circles), as described in Examples 3 and 4.

FIG. 12 depicts the dissolution profile of a packaged modified release formulation over three months at 40° C./75% RH, wherein the modified release particles have 40% LUBRITAB™ in the coating, and the formulation is packaged in a PET/ALU/PE aluminum pouch (9 μm aluminum foil) from Bischof & Klein (Lengerich Germany), as described in Example 6.

FIG. 13 depicts the dissolution profile of a packaged modified release formulation over two months at 40° C./75% RH, wherein the modified release particles have 40% LUBRITAB™ in the coating, and the formulation is packaged in a DUMA™ bottle (30 ml) with 2 g of silica gel desiccant, as described in Example 6.

FIG. 14 plots the dissolution profile of a packaged formulation prior to and after storage for 0 and 18 months at 30° C. and 65% relative humidity in a DUMA™ bottle without desiccant, as described in Example 7.

FIG. 15 plots the dissolution profile of a packaged formulation prior to and after storage for 0 and 18 months at 30° C. and 65% relative humidity in a DUMA™ bottle with 2 g silica gel desiccant in cap, as described in Example 7.

FIG. 16 plots the dissolution profile of a packaged formulation prior to and after storage for 0 and 18 months at 30° C. and 65% relative humidity in a REXAM™ bottle heat sealed without desiccant, as described in Example 7.

FIG. 17 plots the dissolution profile of a packaged formulation prior to and after storage for 0 and 18 months at 30° C. and 65% relative humidity in a Bischof & Klein PET/ALU/PE sachet with 9 μm ALU foil, as described in Example 7.

FIG. 18 plots the mean+SD (standard deviation) plasma gamma-hydroxybutyrate concentrations (microgram/mL) versus time for two different formulations of gamma-hydroxybutyrate tested in vivo according to the methods of Example 8. Time profiles are given for a 4.5 g dose of the second formulation of Example 1 administered once (• symbols) (N=26) and a 4.5 g dose of XYREM® administered in two divided doses (- symbols) (N=15).

FIG. 19 plots the mean+SD (standard deviation) plasma gamma-hydroxybutyrate concentrations (microgram/mL) versus time after a Single Oral Administration of 4.5 g (• symbols) and 6 g (▴ symbols) of the second formulation of Example 1 in the same 7 subjects tested in vivo according to the methods of Example 8.

FIG. 20 plots the mean+SD (standard deviation) plasma gamma-hydroxybutyrate concentrations (microgram/mL) versus time of three separate doses of the second formulation prepared according to Example 1 tested in vivo according to the methods of Example 8. Mean time profiles are given for a single oral administration of 4.5 g (N=26) (•), 6.0 g (N=19) (▴) or 7.5 g (▪) doses (N=11).

FIG. 21 plots the mean plasma gamma-hydroxybutyrate concentrations (microgram/mL) of a single dose of 7.5 g (▪) of the second formulation prepared according to Example 1 compared to 2×4.5 g XYREM® post-fed (Source NDA 21-196 review).

FIGS. 22A and 22B depict a planar view of sachet-type packaging for use in the present invention. The packaging comprises two flat sheets of equal dimension sealed to one another around their periphery in FIG. 22A to define a hollow interior in which the drug product is packaged. In FIG. 22B the packaging is cut across one end so that the drug product can be dispensed.

FIG. 23 depicts the left hand of an individual holding open the sachet depicted in FIG. 22B, with the drug contents exposed and ready to be poured into the cup of water which is also depicted.

FIG. 24 depicts an alternative type of packaging for the drug product of the present invention. The packaging is a bottle constructed of moisture resistant material, and has a screw lid cap removed thereby exposing the drug product inside the bottle.

FIG. 25 depicts the design of the human comparative trial of a formulation manufactured at two different scales as reported in Example 9.

FIG. 26 plots time concentration curves for the gamma-hydroxybutyrate plasma concentrations for formulations reported in Table 9b produced during the human comparative trial reported in Example 9.

FIG. 27A depicts the dissolution profile in 0.1N HCl of a 4.5 g dose of the formulation over 72 months at 25° C./60% RH, as described in Example 10.

FIG. 27B depicts the dissolution profile in pH 6.8 of a 4.5 g dose of the formulation over 72 months at 25° C./60% RH, as described in Example 10.

FIG. 27C depicts the dissolution profile in 0.1N HCl of a 4.5 g dose of the formulation over 24 months at 30° C./65% RH, as described in Example 10.

FIG. 27D depicts the dissolution profile in pH 6.8 of a 4.5 g dose of the formulation over 24 months at 30° C./65% RH, as described in Example 10.

FIG. 27E depicts the dissolution profile in 0.1N HCl of a 4.5 g dose of the formulation over 6 months at 40° C./75% RH, as described in Example 10.

FIG. 27F depicts the dissolution profile in pH 6.8 of a 4.5 g dose of the formulation over 6 months at 40° C./75% RH, as described in Example 10.

FIG. 27G depicts the dissolution profile in 0.1N HCl of a 4.5 g dose of the formulation over 72 months at 25° C./60% RH, as described in Example 10.

FIG. 27H depicts the dissolution profile in pH 6.8 of a 4.5 g dose of the formulation over 72 months at 25° C./60% RH, as described in Example 10.

FIG. 27I depicts the dissolution profile in 0.1N HCl of a 4.5 g dose of the formulation over 24 months at 30° C./65% RH, as described in Example 10.

FIG. 27J depicts the dissolution profile in pH 6.8 of a 4.5 g dose of the formulation over 24 months at 30° C./65% RH, as described in Example 10.

FIG. 27K depicts the dissolution profile in 0.1N HCl of a 4.5 g dose of the formulation over 6 months at 40° C./75% RH, as described in Example 10.

FIG. 27L depicts the dissolution profile in pH 6.8 of a 4.5 g dose of the formulation over 6 months at 40° C./75% RH, as described in Example 10.

FIG. 27M depicts the dissolution profile in 0.1N HCl of a 4.5 g dose of the formulation over 72 months at 25° C./60% RH, as described in Example 10.

FIG. 27N depicts the dissolution profile in pH 6.8 of a 4.5 g dose of the formulation over 72 months at 25° C./60% RH, as described in Example 10.

FIG. 27O depicts the dissolution profile in 0.1N HCl of a 4.5 g dose of the formulation over 24 months at 30° C./65% RH, as described in Example 10.

FIG. 27P depicts the dissolution profile in pH 6.8 of a 4.5 g dose of the formulation over 24 months at 30° C./65% RH, as described in Example 10.

FIG. 27Q depicts the dissolution profile in 0.1N HCl of a 4.5 g dose of the formulation over 6 months at 40° C./75% RH, as described in Example 10.

FIG. 27R depicts the dissolution profile in pH 6.8 of a 4.5 g dose of the formulation over 6 months at 40° C./75% RH, as described in Example 10.

FIG. 28A depicts the dissolution profile in 0.1N HCl of a 6.0 g dose of the formulation over 72 months at 25° C./60% RH, as described in Example 11.

FIG. 28B depicts the dissolution profile in pH 6.8 of a 6.0 g dose of the formulation over 72 months at 25° C./60% RH, as described in Example 11.

FIG. 28C depicts the dissolution profile in 0.1N HCl of a 6.0 g dose of the formulation over 10 months at 30° C./65% RH, as described in Example 11.

FIG. 28D depicts the dissolution profile in pH 6.8 of a 6.0 g dose of the formulation over 10 months at 30° C./65% RH, as described in Example 11.

FIG. 28E depicts the dissolution profile in 0.1N HCl of a 6.0 g dose of the formulation over 6 months at 40° C./75% RH, as described in Example 11.

FIG. 28F depicts the dissolution profile in pH 6.8 of a 6.0 g dose of the formulation over 6 months at 40° C./75% RH, as described in Example 11.

FIG. 29A depicts the dissolution profile in 0.1N HCl of a 7.5 g dose of the formulation over 72 months at 25° C./60% RH, as described in Example 12.

FIG. 29B depicts the dissolution profile in pH 6.8 of a 7.5 g dose of the formulation over 72 months at 25° C./60% RH, as described in Example 12.

FIG. 29C depicts the dissolution profile in 0.1N HCl of a 7.5 g dose of the formulation over 15 months at 30° C./65% RH, as described in Example 12.

FIG. 29D depicts the dissolution profile in pH 6.8 of a 7.5 g dose of the formulation over 15 months at 30° C./65% RH, as described in Example 12.

FIG. 29E depicts the dissolution profile in 0.1N HCl of a 7.5 g dose of the formulation over 6 months at 40° C./75% RH, as described in Example 12.

FIG. 29F depicts the dissolution profile in pH 6.8 of a 7.5 g dose of the formulation over 6 months at 40° C./75% RH, as described in Example 12.

FIG. 30A depicts the dissolution profile in 0.1N HCl of a 9.0 g dose of the formulation over 72 months at 25° C./60% RH, as described in Example 13.

FIG. 30B depicts the dissolution profile in pH 6.8 of a 9.0 g dose of the formulation over 72 months at 25° C./60% RH, as described in Example 13.

FIG. 30C depicts the dissolution profile in 0.1N HCl of a 9.0 g dose of the formulation over 18 months at 30° C./65% RH, as described in Example 13.

FIG. 30D depicts the dissolution profile in pH 6.8 of a 9.0 g dose of the formulation over 18 months at 30° C./65% RH, as described in Example 13.

FIG. 30E depicts the dissolution profile in 0.1N HCl of a 9.0 g dose of the formulation over 6 months at 40° C./75% RH, as described in Example 13.

FIG. 30F depicts the dissolution profile in pH 6.8 of a 9.0 g dose of the formulation over 6 months at 40° C./75% RH, as described in Example 13.

FIG. 30G depicts the dissolution profile in 0.1N HCl of a 9.0 g dose of the formulation over 72 months at 25° C./60% RH, as described in Example 13.

FIG. 30H depicts the dissolution profile in pH 6.8 of a 9.0 g dose of the formulation over 72 months at 25° C./60% RH, as described in Example 13.

FIG. 30I depicts the dissolution profile in 0.1N HCl of a 9.0 g dose of the formulation over 18 months at 30° C./65% RH, as described in Example 13.

FIG. 30J depicts the dissolution profile in pH 6.8 of a 9.0 g dose of the formulation over 18 months at 30° C./65% RH, as described in Example 13.

FIG. 30K depicts the dissolution profile in 0.1N HCl of a 9.0 g dose of the formulation over 6 months at 40° C./75% RH, as described in Example 13.

FIG. 30L depicts the dissolution profile in pH 6.8 of a 9.0 g dose of the formulation over 6 months at 40° C./75% RH, as described in Example 13.

FIG. 30M depicts the dissolution profile in 0.1N HCl of a 9.0 g dose of the formulation over 72 months at 25° C./60% RH, as described in Example 13.

FIG. 30N depicts the dissolution profile in pH 6.8 of a 9.0 g dose of the formulation over 72 months at 25° C./60% RH, as described in Example 13.

FIG. 30O depicts the dissolution profile in 0.1N HCl of a 9.0 g dose of the formulation over 18 months at 30° C./65% RH, as described in Example 13.

FIG. 30P depicts the dissolution profile in pH 6.8 of a 9.0 g dose of the formulation over 18 months at 30° C./65% RH, as described in Example 13.

FIG. 30Q depicts the dissolution profile in 0.1N HCl of a 9.0 g dose of the formulation over 6 months at 40° C./75% RH, as described in Example 13.

FIG. 30R depicts the dissolution profile in pH 6.8 of a 9.0 g dose of the formulation over 6 months at 40° C./75% RH, as described in Example 13.

### DETAILED DESCRIPTION OF THE INVENTION

The present invention may be understood more readily by reference to the following detailed description of particular non-limiting embodiments of the invention described herein and the Examples included therein.

### Definitions and Use of Terms

Wherever an analysis or test is required to understand a given property or characteristic recited herein, it will be understood that the analysis or test is performed in accordance with applicable guidances, draft guidances, regulations and monographs of the United States Food and Drug Administration (“FDA”) and United States Pharmacopoeia (“USP”)/National Formulary (“NF”) applicable to drug products in the United States in force as of Oct. 1, 2017 unless otherwise specified.

When a pharmacokinetic comparison is made between a formulation described or claimed herein and a reference product, it will be understood that the comparison is preferably performed in a suitable designed cross-over trial, although it will also be understood that a cross-over trial is not required unless specifically stated. It will also be understood that the comparison can be made either directly or indirectly. For example, even if a formulation has not been tested directly against a reference formulation, it can still satisfy a comparison to the reference formulation if it has been tested against a different formulation, and the comparison with the reference formulation can be deduced therefrom.

As used in this specification and in the claims which follow, the singular forms “a,” “an” and “the” include plural referents unless the context dictates otherwise. Thus, for example, reference to “an ingredient” includes mixtures of ingredients, reference to “an active pharmaceutical agent” includes more than one active pharmaceutical agent, and the like.

When ranges are given by specifying the lower end of a range separately from the upper end of the range, it will be understood that the range can be defined by selectively combining any one of the lower end variables with any one of the upper end variables that is mathematically and physically possible. Thus, for example, if a formulation may contain from 1 to 10 weight parts of a particular ingredient, or 2 to 8 parts of a particular ingredient, it will be understood that the formulation may also contain from 2 to 10 parts of the ingredient. In like manner, if a formulation may contain greater than 1 or 2 weight parts of an ingredient and up to 10 or 9 weight parts of the ingredient, it will be understood that the formulation may contain 1-10 weight parts of the ingredient, 2-9 weight parts of the ingredient, etc. unless otherwise specified, the boundaries of the range (lower and upper ends of the range) are included in the claimed range.

In like manner, when various sub-embodiments of a principal embodiment are described herein, it will be understood that the sub-embodiments for the principal embodiment can be combined to define another sub-embodiment. Thus, for example, when a principal embodiment includes sub-embodiments 1, 2 and 3, it will be understood that the principal embodiment can be further limited by any one of sub-embodiments 1, 2 and 3, or any combination of sub-embodiments 1, 2 and 3 that is mathematically and physically possible. In like manner, it will be understood that the principal embodiments described herein can be combined in any manner that is mathematically and physically possible, and that the invention extends to such combinations.

When used herein the term “about” or “substantially” or “approximately” will compensate for variability allowed for in the pharmaceutical industry and inherent in pharmaceutical products, such as differences in product strength due to manufacturing variation and time-induced product degradation. The term allows for any variation which in the practice of pharmaceuticals would allow the product being evaluated to be considered bioequivalent to the recited strength, as described in FDA's March 2003 Guidance for Industry on BIOAVAILABILITY AND BIOEQUIVALENCE STUDIES FOR ORALLY ADMINISTERED DRUG PRODUCTS—GENERAL CONSIDERATIONS.

“Bioavailability” means the rate and extent to which the active ingredient or active moiety is absorbed from a drug product and becomes available at the site of action.

“Relative bioavailability” or “Rel BA” or “RBA” means the percentage of mean AUCinf of the tested product relative to the mean AUCinf of the reference product. Unless otherwise specified, relative bioavailability refers to the percentage of the mean AUCinf observed for a full dose of the test product relative to the mean AUCinf observed for two ½- doses of an immediate release liquid solution administered four hours apart.

In all pharmacokinetic testing described herein, unless otherwise stated, the dosage form, or the initial dosage form if the dosing regimen calls for more than one administration, is administered approximately two hours after consumption of a standardized dinner consisting of 25.5% fat, 19.6% protein, and 54.9% carbohydrates.

The term “chemically stable” means that the GHB in a formulation under consideration does not exhibit unacceptable chemical degradation during storage. Thus, for example, a formulation would be considered chemically stable if, after 72 months, 60 months, 48 months, 36 months, 24 months, 18 months, 12 months, 10 months, 6 months, or 3 months of storage at 25° C./60% RH, 30° C./65% RH, or 40° C./75% RH, the formulation does not contain greater than 3% GHB degradation products. In one particular embodiment, a packaged formulation is chemically stable if the package prevents no more than 0.4% w/w, 0.2% w/w, 0.1% w/w, or 0.05% w/w of the gamma-hydroxybutyrate in the composition from converting to gamma-butyrolactone (GBL) when stored up to 72 months, 60 months, 48 months, 36 months, 24 months, 18 months, 12 months, 10 months, 6 months, or 3 months at 25° C./60% RH, 30° C./65% RH, or 40° C./75% RH. In other examples, the formulation is stable if it has pH<5.9, <0.18% w/w GBL, 90.0% to 110.0% of the amount of gamma-hydroxybutyrate in the pharmaceutical formulation before storage, ≤4.0% w/w water content, and/or a stable dissolution profile after storage for more than 3 months, more than 6 months, more than 12 months, more than 18 months, more than 24 months, more than 36 months, more than 48 months, more than 60 months, or more than 70 months.

“Dissolution stability” refers to a formulation's ability to maintain its stability profile over time. Examples of ways to measure dissolution stability, and criteria which can be used to evaluate dissolution stability, are given in Example 2 hereto. Dissolution stability is also shown in Examples 10-13. In one embodiment, a formulation is considered to have a stable dissolution profile if, after a two-month 40° C./75% relative humidity storage period the composition exhibits a lag time that is less than 70, 60, or 50 minutes different than the lag time at the beginning of the storage period, wherein the lag time is determined from testing in a dissolution apparatus 2 according to USP 38<711> in 900 mL of 0.1N hydrochloric acid at a temperature of 37° C. and a paddle speed of 75 rpm. In another embodiment, a formulation is considered to have a stable dissolution profile if the percentage of gamma-hydroxybutyrate dissolved after a two-month 40° C./75% relative humidity storage period at all tested time points or at 4, 6 or 8 consecutive hourly time points is less than 10% different than the percentage of gamma-hydroxybutyrate dissolved before the storage period at the same time points when tested in a dissolution apparatus 2 according to USP 38<711> in 900 mL of 0.1N hydrochloric acid at a temperature of 37° C. and a paddle speed of 75 rpm. In another embodiment, a formulation is considered to have a stable dissolution profile if the percentage of gamma-hydroxybutyrate dissolved after storage for up to 72 months, 60 months, 48 months, 36 months, 24 months, 18 months, 12 months, 10 months, 6 months, or 3 months at 25° C./60% RH, 30° C./65% RH, or 40° C./75% RH at all tested time points or at 1, 4, 6, 8, or 12 hourly time points is less than 10% different than the percentage of gamma-hydroxybutyrate dissolved before the storage period at the same time points when tested in a dissolution apparatus 2 according to USP 38<711> in 900 mL of 0.1N hydrochloric acid at a temperature of 37° C. and a paddle speed of 75 rpm.

When used herein the term “gamma-hydroxybutyrate” or GHB, including hydrates, solvates, complexes and tautomers. Gamma-hydroxybutyric acid salts can be selected from the sodium salt of gamma-hydroxybutyric acid (i.e. sodium oxybate), the potassium salt of gamma-hydroxybutyric acid, the magnesium salt of gamma-hydroxybutyric acid, the calcium salt of gamma-hydroxybutyric acid, the lithium salt of gamma-hydroxybutyric, the tetra ammonium salt of gamma-hydroxybutyric acid or any other pharmaceutically acceptable salt form.

“Lag time” refers to the latency period for release of gamma-hydroxybutyrate from a given formulation determined according to the method described in Example 2 hereto.

“Packaging” or “package” refers to any packaging material suitable for packaging either unit dose of bulk pharmaceutical products. The term thus includes bottles (glass and plastic), barrels, bags, vials, ampules, blister packs, sachets, stick-packs, and other containers. The size, type and physical characteristics of the packaging or package are limited only by the compatibility of the packaging with the pharmaceutical product contained therein, and the distribution requirements for the packaging.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use. The term “formulation” or “composition” refers to the quantitative and qualitative characteristics of a drug product or dosage form prepared in accordance with the current invention.

As used herein the doses and strengths of gamma-hydroxybutyrate are expressed in equivalent-gram (g) weights of sodium oxybate unless stated expressly to the contrary. Thus, when considering a dose of gamma-hydroxybutyrate other than the sodium salt of gamma-hydroxybutyrate, one must convert the recited dose or strength from sodium oxybate to the gamma-hydroxybutyrate under evaluation. Thus, if an embodiment is said to provide a 4.5 g dose of gamma-hydroxybutyrate, because the form of gamma-hydroxybutyrate is not specified, it will be understood that the dose encompasses a 4.5 g dose of sodium oxybate, a 5.1 g dose of potassium gamma-hydroxybutyrate (assuming a 126.09 g/mol MW for sodium oxybate and a 142.20 g/mol MW for potassium gamma-hydroxybutyrate), and a 3.7 g dose of the free base (assuming a 126.09 g/mol MW for sodium oxybate and a 104.1 g/mol MW for the free base of gamma-hydroxybutyrate), or by the weight of any mixture of salts of gamma-hydroxybutyric acid that provides the same amount of GHB as 4.5 g of sodium oxybate.

As used herein “microparticle” means any discreet particle of solid material. The particle can be made of a single material or have a complex structure with core and shells and be made of several materials. The terms “microparticle,” “particle,” “microspheres” or “pellet” are interchangeable and have the same meaning. Unless otherwise specified, the microparticle has no particular particle size or diameter and is not limited to particles with volume mean diameter D(4,3) below 1 mm.

As used herein, the “volume mean diameter D(4,3)” is calculated according to the following formula:

$$D ( 4 , 3 ) = \sum ( {d}^{4} i . ni ) / \sum ( {d}^{3} i . ni )$$

wherein the diameter d of a given particle is the diameter of a hard sphere having the same volume as the volume of that particle.

As used herein, the terms “RH” and “relative humidity” are used interchangeably.

As used herein, the terms “finished composition,” “finished formulation” or “formulation” are interchangeable and designate the modified release formulation of gamma-hydroxybutyrate preferably comprising modified release microparticles of gamma-hydroxybutyrate, immediate release microparticles of gamma-hydroxybutyrate, and any other excipients. A “composition” can always be a “finished composition.”

As used herein and in the claims that follow, an “immediate release (IR) component” or “immediate release portion” of a formulation includes physically discreet portions of a formulation, mechanistically discreet portions of a formulation, and discreet portions of a formulation that lend to or support a defined IR dissolution characteristic. Thus, for example, any formulation that releases active ingredient at the rate and extent required of the immediate release component of the formulations of the present invention includes an “immediate release component,” even if the immediate release component is physically integrated in what might otherwise be considered an extended release formulation. Thus, the IR component can be structurally discreet or structurally indiscreet from (i.e. integrated with) the MR component. In a particular embodiment, the IR component and MR component are provided as particles, and in an even more particular subembodiments the IR component and MR component are provided as particles discreet from each other.

As used here in, “immediate release formulation”, “immediate release component” or “immediate release portion” refers to a composition that releases at least 80% of its gamma-hydroxybutyrate in 1 hour when tested in a dissolution apparatus 2 according to USP 38<711> in a 0.1N HCl dissolution medium at a temperature of 37° C. and a paddle speed of 75 rpm.

In like manner, a “modified-release (MR) component” or “modified release portion” includes that portion of a formulation or dosage form that lends to or supports a particular MR characteristic, regardless of the physical formulation in which the MR component is integrated. The modified release drug delivery systems are designed to deliver drugs at a specific time or over a period of time after administration, or at a specific location in the body. The USP defines a modified release system as one in which the time course or location of drug release or both, are chosen to accomplish objectives of therapeutic effectiveness or convenience not fulfilled by conventional IR dosage forms. More specifically, MR solid oral dosage forms include extended release (ER) and delayed-release (DR) products. A DR product is one that releases a drug all at once at a time other than promptly after administration. Typically, coatings (e.g., enteric coatings) are used to delay the release of the drug substance until the dosage form has passed through the acidic medium of the stomach. An ER product is formulated to make the drug available over an extended period after ingestion, thus allowing a reduction in dosing frequency compared to a drug presented as a conventional dosage form, e.g. a solution or an immediate release dosage form. For oral applications, the term “extended-release” is usually interchangeable with “sustained-release,” “prolonged-release” or “controlled-release.”

Traditionally, extended-release systems provided constant drug release to maintain a steady concentration of drug. For some drugs, however, zero-order delivery may not be optimal and more complex and sophisticated systems have been developed to provide multi-phase delivery. One can distinguish among four categories of oral MR delivery systems: (1) delayed-release using enteric coatings, (2) site-specific or timed release (e.g. for colonic delivery), (3) extended-release (e.g., zero-order, first-order, biphasic release, etc.), and (4), programmed release (e.g., pulsatile, delayed extended release, etc.) See Modified Oral Drug Delivery Systems at page 34 in Gibaldi's DRUG DELIVERY SYSTEMS IN PHARMACEUTICAL CARE, AMERICAN SOCIETY OF HEALTH-SYSTEM PHARMACISTS, 2007 and Rational Design of Oral Modified-release Drug Delivery Systems at page 469 in DEVELOPING SOLID ORAL DOSAGE FORMS: PHARMACEUTICAL THEORY AND PRACTICE, Academic Press, Elsevier, 2009. As used herein, “modified release formulation” or “modified release component” in one embodiment refers to a composition that releases its gamma-hydroxybutyrate according a multiphase delivery that is comprised in the fourth class of MR products, e.g. delayed extended release. As such it differs from the delayed release products that are classified in the first class of MR products.

As used herein the terms “coating,” “coating layer,” “coating film,” “film coating” and like terms are interchangeable and have the same meaning. The terms refer to the coating applied to a particle comprising gamma-hydroxybutyrate that controls the modified release of the gamma-hydroxybutyrate.

Type 1 Narcolepsy (NT1) refers to narcolepsy characterized by excessive daytime sleepiness (“EDS”) and cataplexy. Type 2 Narcolepsy (NT2) refers to narcolepsy characterized by excessive daytime sleepiness without cataplexy. A diagnosis of narcolepsy (with or without cataplexy) can be confirmed by one or a combination of (i) an overnight polysomnogram (PSG) and a Multiple Sleep Latency Test (MSLT) performed within the last 2 years, (ii) a full documentary evidence confirming diagnosis from the PSG and MSLT from a sleep laboratory must be made available, (iii) current symptoms of narcolepsy including: current complaint of EDS for the last 3 months (Epworth Sleepiness Scale (ESS) greater than 10), (iv) mean Maintenance of Wakefulness Test (MWT) less than 8 minutes, (v) mean number of cataplexy events of 8 per week on baseline Sleep/Cataplexy Diary, and/or (vi) presence of cataplexy for the last 3 months and 28 events per week during screening period.

Unless otherwise specified herein, percentages, ratios and numeric values recited herein are based on weight; averages and means are arithmetic means.

It will be understood, when defining a composition by its dissolution properties herein, that the formulation can in the alternative be defined as “means for” achieving the recited dissolution properties. Thus, a formulation in which the modified release component releases less than 20% of its gamma-hydroxybutyrate at one hour can instead be defined as a formulation comprising “means for” or “modified release means for” releasing less than 20% of its gamma-hydroxybutyrate at one hour. It will be further understood that the preferred structures for achieving the recited dissolution properties are the structures described in the examples hereof that accomplish the recited dissolution properties.

### Discussion of Principal Embodiments

The invention can be described in terms of principal embodiments, which in turn can be recombined to make other principal embodiments, and limited by sub-embodiments to make other principal embodiments.

In one principal embodiment, the invention provides a packaged pharmaceutical composition having a stable dissolution profile, wherein the pharmaceutical composition comprises immediate release and modified release components of gamma-hydroxybutyrate or a pharmaceutically acceptable salt thereof.

In another principal embodiment, the invention provides a packaged pharmaceutical composition comprising a modified release gamma-hydroxybutyrate pharmaceutical composition within a package, wherein the pharmaceutical composition comprises immediate release and modified release components of gamma-hydroxybutyrate or a pharmaceutically acceptable salt thereof, and the package has an interior volume having a relative humidity from 29% to 54%. In some iterations, the relative humidity of the package is from 29% to 54% for a period of at least 2 months when stored at 40° C. and 75% relative humidity. In other iterations, the relative humidity of the package is greater than 29% at 1 week and less than 54% at 2 months when stored at 40° C. and 75% relative humidity. In further iterations, the relative humidity of the package is from 35% to 39% after one week and from 39% to 48% after 2 months when stored at 40° C. and 75% relative humidity. In still other iterations, no more than 0.4% of gamma-hydroxybutyrate in the pharmaceutical composition is converted to gamma-butyrolactone (GBL) when stored two months at 40° C. and 75% relative humidity. In other iterations, the package has a water vapor transmission rate of less than 7 mg/day/liter when measured according to USP 38<671>.

In some iterations, after a two-month 40° C./75% relative humidity storage period, the pharmaceutical composition exhibits a lag time that is less than 70 minutes different than the lag time at the beginning of the storage period, wherein the lag time is determined from testing in a dissolution apparatus 2 according to USP 38 <711> in 900 mL of 0.1N hydrochloric acid at a temperature of 37° C. and a paddle speed of 75 rpm. In other iterations, after a two-month 40° C./75% relative humidity storage period, the pharmaceutical composition has a dissolution of gamma-hydroxybutyrate that differs by less than 10% than the dissolution of gamma-hydroxybutyrate before the storage period when tested for at least four consecutive hourly time points in a dissolution apparatus 2 according to USP 38<711> in 900 mL of 0.1N hydrochloric acid at a temperature of 37° C. and a paddle speed of 75 rpm.

In another embodiment, after storage for up to 72 months, 60 months, 48 months, 36 months, 24 months, 18 months, 12 months, 10 months, 6 months, or 3 months at 25° C./60% RH, 30° C./65% RH, or 40° C./75% RH, the pharmaceutical formulation has a dissolution of gamma-hydroxybutyrate that differs by less than 10%, 9%, 8%, 7%, 6%, 5%, 4%, 3%, 2% or 1% than the dissolution of gamma-hydroxybutyrate before the storage period when tested for at least three time points by a 0.1 N HCl dissolution test by HPLC. In an example, after storage for up to 72 months, 60 months, 48 months, 36 months, 24 months, 18 months, 12 months, 10 months, 6 months, or 3 months at 25° C./60% RH, 30° C./65% RH, or 40° C./75% RH, the pharmaceutical formulation has a dissolution of gamma-hydroxybutyrate of between about 40% and about 60% after 1 hour of dissolution in 0.1 N HCl, between about 45% and about 65% after 4 hours of dissolution in 0.1 N HCl, and greater than or equal to 80% dissolution after 12 hours of dissolution in 0.1 N HCl. In another example, after storage for up to 72 months, 60 months, 48 months, 36 months, 24 months, 18 months, 12 months, 10 months, 6 months, or 3 months at 25° C./60% RH, 30° C./65% RH, or 40° C./75% RH, the pharmaceutical formulation has a dissolution of gamma-hydroxybutyrate of ≥85%, ≥90%, ≥95%, or ≥98% dissolution after 1 hour by a pH 6.8 dissolution test by HPLC.

In yet another principal embodiment, the invention provides a packaged pharmaceutical composition having a stable dissolution profile, comprising a pharmaceutical composition within a package, wherein the pharmaceutical composition comprises immediate release and modified release components of gamma-hydroxybutyrate or a pharmaceutically acceptable salt thereof, and wherein the modified release component comprises a core comprising gamma-hydroxybutyrate or a pharmaceutically acceptable salt thereof and a coating comprising a hydrophobic compound and a mixture of methacrylic acid copolymers. In some iterations, the hydrophobic compound is glyceryl tristearate or hydrogenated vegetable oil, and the mixture of methacrylic acid copolymers comprises methacrylic acid and ethyl acrylate copolymer NF and methacrylic acid and methyl methacrylate copolymer (1:2) NF. In certain iterations, the coating comprises from 40 to 70 weight parts of the hydrophobic compound and from 30 to 60 weight parts of the mixture of methacrylic acid copolymers. In other iterations, the weight ratio of the hydrophobic compound to the mixture of methacrylic acid copolymers is about 1.5:1. In yet other iterations, the hydrophobic compound and the mixture of methacrylic polymers are greater than 90% of the weight of the coating. In still other iterations, the coating is from 10 to 50% of the weight of the modified release component. In other iterations, the mixture of methacrylic acid copolymers is substantially ionized at pH 7.5. In alternate iterations, the hydrophobic compound comprises hydrogenated vegetable oil. In still other iterations, the hydrophobic compound has a melting point equal to or greater than 40° C. and the mixture of methacrylic acid copolymers have a pH trigger greater than 5.6. In further iterations, the modified release component does not contain a barrier coat between the core containing the gamma hydroxybutyrate and the coating. In particular embodiments, and the modified release component comprises particles having an average diameter of from 200 to 800 microns. In certain iterations, the immediate release component comprises particles. For example, the immediate release component comprises particles having an average diameter from 95 to 600 microns. In additional iterations, the pharmaceutical composition can further comprise an acidifying agent and a suspending or viscosifying agent as detailed below.

In a further principal embodiment, the invention provides a packaged solid particulate pharmaceutical composition having a stable dissolution profile over time, comprising immediate release and modified release components of gamma-hydroxybutyrate or a pharmaceutically acceptable salt thereof, wherein: (a) the modified release component comprises: (i) a core comprising gamma-hydroxybutyrate or a pharmaceutically acceptable salt thereof; and (ii) a coating comprising a hydrophobic compound selected from glyceryl tristearate and hydrogenated vegetable oil and a mixture of methacrylic acid copolymers comprising methacrylic acid and ethyl acrylate copolymer NF and methacrylic acid and methyl methacrylate copolymer (1:2) NF; and (b) the relative humidity inside the packaging is in a range of from 29% to 54% after one week at 40° C. and 75% relative humidity and the package maintains the relative humidity within a range of from 29% to 54% for a period of at least 2 months when stored at 40° C. and 75% relative humidity.

In another principal embodiment, the invention provides a packaged solid particulate pharmaceutical composition having a stable dissolution profile over time, comprising immediate release and modified release components of gamma-hydroxybutyrate or a pharmaceutically acceptable salt thereof, wherein: (a) the modified release component comprises: (i) a core comprising gamma-hydroxybutyrate or a pharmaceutically acceptable salt thereof; and (ii) a coating comprising a hydrophobic compound selected from glyceryl tristearate and hydrogenated vegetable oil and a mixture of methacrylic acid copolymers comprising methacrylic acid and ethyl acrylate copolymer NF and methacrylic acid and methyl methacrylate copolymer (1:2) NF; and (b) after a two-month 40° C./75% relative humidity storage period the composition exhibits a lag time that is less than 70, 60, or 50 minutes different than the lag time at the beginning of the storage period, wherein the lag time is determined from testing in a dissolution apparatus 2 according to USP 38<711> in 900 mL of 0.1N hydrochloric acid at a temperature of 37° C. and a paddle speed of 75 rpm.

In yet another principal embodiment, the invention provides a packaged solid particulate pharmaceutical composition having a stable dissolution profile over time, comprising immediate release and modified release components of gamma-hydroxybutyrate or a pharmaceutically acceptable salt thereof, wherein: (a) the modified release component comprises: (i) a core comprising gamma-hydroxybutyrate or a pharmaceutically acceptable salt thereof; and (ii) a coating comprising a hydrophobic compound selected from glyceryl tristearate and hydrogenated vegetable oil and a mixture of methacrylic acid copolymers comprising methacrylic acid and ethyl acrylate copolymer NF and methacrylic acid and methyl methacrylate copolymer (1:2) NF; and (b) the percentage of gamma-hydroxybutyrate dissolved after a two-month 40° C./75% relative humidity storage period at all time points tested, or 4, 6 or 8 consecutive hourly time points, is less than 10% different than the percentage of gamma-hydroxybutyrate dissolved before the storage period at the same 4, 6 or 8 consecutive hourly time points when tested in a dissolution apparatus 2 according to USP 38<711> in 900 mL of 0.1N hydrochloric acid at a temperature of 37° C. and a paddle speed of 75 rpm.

In still another principal embodiment, the invention provides a packaged solid particulate pharmaceutical composition having a stable dissolution profile over time, comprising immediate release and modified release components of gamma-hydroxybutyrate or a pharmaceutically acceptable salt thereof, wherein: (a) the modified release component comprises: (i) a core comprising gamma-hydroxybutyrate or a pharmaceutically acceptable salt thereof; and (ii) a coating comprising a hydrophobic compound selected from glyceryl tristearate and hydrogenated vegetable oil and a mixture of methacrylic acid copolymers comprising methacrylic acid and ethyl acrylate copolymer NF and methacrylic acid and methyl methacrylate copolymer (1:2) NF; and (b) the package has a water vapor transmission rate of less than 7 mg/day/liter when measured according to USP 38<671>.

In an alternate embodiment, the invention provides a packaged solid particulate pharmaceutical composition having a stable dissolution profile over time, comprising immediate release and modified release components of gamma-hydroxybutyrate or a pharmaceutically acceptable salt thereof, wherein: (a) the modified release component comprises: (i) a core comprising gamma-hydroxybutyrate or a pharmaceutically acceptable salt thereof; and (ii) a coating comprising a hydrophobic compound selected from glyceryl tristearate and hydrogenated vegetable oil and a mixture of methacrylic acid copolymers comprising methacrylic acid and ethyl acrylate copolymer NF and methacrylic acid and methyl methacrylate copolymer (1:2) NF; (b) the package has a water vapor transmission rate of less than 7 mg/day/liter when measured according to USP 38<671>; and (c) the package prevents no more than 0.4% of the gamma-hydroxybutyrate in the composition from converting to gamma-butyrolactone (GBL) when stored two months at 40° C. and 75% relative humidity.

In a further embodiment, the invention provides a solid particulate pharmaceutical composition having a stable dissolution profile over time comprising immediate release and modified release components of gamma-hydroxybutyrate or a pharmaceutically acceptable salt thereof, wherein the modified release component comprises: (a) a core comprising gamma-hydroxybutyrate or a pharmaceutically acceptable salt thereof; and (b) a coating comprising a hydrophobic compound selected from glyceryl tristearate and hydrogenated vegetable oil and a mixture of methacrylic acid copolymers comprising methacrylic acid and ethyl acrylate copolymer NF and methacrylic acid and methyl methacrylate copolymer (1:2) NF.

In still another embodiment, the invention provides a solid particulate pharmaceutical composition having a stable dissolution profile over time comprising immediate release and modified release components of gamma-hydroxybutyrate or a pharmaceutically acceptable salt thereof, wherein: (a) the modified release component comprises: (i) a core comprising gamma-hydroxybutyrate or a pharmaceutically acceptable salt thereof; and (ii) a coating comprising a hydrophobic compound selected from glyceryl tristearate and hydrogenated vegetable oil and a mixture of methacrylic acid copolymers comprising methacrylic acid and ethyl acrylate copolymer NF and methacrylic acid and methyl methacrylate copolymer (1:2) NF; and (b) after a two-month 40° C./75% relative humidity storage period the composition exhibits a lag time that is less than 70, 60, or 50 minutes different than the lag time at the beginning of the storage period, wherein the lag time is determined from testing in a dissolution apparatus 2 according to USP 38<711> in 900 mL of 0.1N hydrochloric acid at a temperature of 37° C. and a paddle speed of 75 rpm.

In yet another embodiment, the invention provides a solid particulate pharmaceutical composition having a stable dissolution profile over time comprising immediate release and modified release components of gamma-hydroxybutyrate or a pharmaceutically acceptable salt thereof, wherein: (a) the modified release component comprises: (i) a core comprising gamma-hydroxybutyrate or a pharmaceutically acceptable salt thereof; and (ii) a coating comprising a hydrophobic compound selected from glyceryl tristearate and hydrogenated vegetable oil and a mixture of methacrylic acid copolymers comprising methacrylic acid and ethyl acrylate copolymer NF and methacrylic acid and methyl methacrylate copolymer (1:2) NF; and (b) the percentage of gamma-hydroxybutyrate dissolved after a two-month 40° C./75% relative humidity storage period at 4, 6 or 8 consecutive hourly time points is less than 10% different than the percentage of gamma-hydroxybutyrate dissolved before the storage period at the same 4, 6 or 8 consecutive hourly time points when tested in a dissolution apparatus 2 according to USP 38<711> in 900 mL of 0.1N hydrochloric acid at a temperature of 37° C. and a paddle speed of 75 rpm.

In still other embodiments, the invention provides methods of using the packaged pharmaceutical composition to treat narcolepsy Type 1 or Type 2. The packaged pharmaceutical composition may further be used to treat symptoms of narcolepsy, including but not limited to excessive daytime sleepiness (EDS), cataplexy (a sudden loss of muscle tone triggered by strong emotions, seen in approximately 60% of patients), hypnogogic hallucination (HH), sleep paralysis (SP), and disturbed nocturnal sleep (DNS). The composition is also effective to induce sleep for six to eight, most preferably eight consecutive hours. The methods comprise administering the pharmaceutical composition to an individual in need thereof. The individual may be a human subject. In general, the methods comprise opening the package comprising the gamma-hydroxybutyrate composition, mixing (e.g., via shaking, stirring, or otherwise agitating) the solid pharmaceutical composition with liquid (e.g., water) to form a mixture, and orally administering the mixture to the individual.

Surprisingly, the pharmaceutical formulation may be stored for up to at least 72 months prior to orally administering. For example, the formulation may be stable and suitable for administration after storage for more than 3 months, more than 6 months, more than 12 months, more than 18 months, more than 24 months, more than 36 months, more than 48 months, more than 60 months, or more than 70 months. The pharmaceutical formulation may be stored for 3 months to 72 months prior to orally administering and remain stable (e.g. pH<5.9, <0.18% w/w GBL, 90.0% to 110.0% of the amount of gamma-hydroxybutyrate in the pharmaceutical formulation before storage, ≤4.0% w/w water content, and stable dissolution profile) such that the formulation is still suitable for administration after storage within a packaging at temperatures ranging from about 25° C. to about 40° C. and relative humidity ranging from about 60% to about 75%.

In some embodiments, a pharmaceutical formulation may be stored within a package, the pharmaceutical formulation comprising: an immediate release portion comprising gamma-hydroxybutyrate or a pharmaceutically acceptable salt thereof; and a modified release portion comprising gamma-hydroxybutyrate or a pharmaceutically acceptable salt thereof. The pharmaceutical formulation may further include one or more suspending or viscosifying agents selected from xanthan gum, medium viscosity sodium carboxymethyl cellulose, mixtures of microcrystalline cellulose and sodium carboxymethyl cellulose, mixtures of microcrystalline cellulose and guar gum, medium viscosity hydroxyethyl cellulose, agar, sodium alginate, mixtures of sodium alginate and calcium alginate, gellan gum, carrageenan gum, medium viscosity hydroxypropylmethyl cellulose, and mixtures thereof. In other embodiments, the pharmaceutical formulation further includes an acidifying agent selected from malic acid, citric acid, tartaric acid, adipic acid, boric acid, maleic acid, phosphoric acid, ascorbic acid, oleic acid, capric acid, caprylic acid, or benzoic acid. In some examples, the suspending or viscosifying agent and the acidifying agent are separate and distinct from the immediate release portion and the modified release portion. The dose of gamma-hydroxybutyrate in the pharmaceutical formulation stored in each package may be an amount equivalent to 4.5 g, 6.0 g, 7.5 g, or 9.0 g sodium oxybate.

The pharmaceutical formulation may remain stable during storage in the package such that the pharmaceutical formulation may still be administered to a patient after storage for an extended period of time. An extended period of time may be any time between 2 months and 72 months. For example, the pharmaceutical formulation may be suitable for administration to the patient after being stored in the package for up to about 72 months, about 71 months, about 70 months, about 69 months, about 68 months, about 67 months, about 66 months, about 65 months, about 64 months, about 63 months, about 62 months, about 60 months, about 59 months, about 58 months, about 57 months, about 56 months, about 55 months, about 54 months, about 53 months, about 52 months, about 51 months, about 50 months, about 49 months, about 48 months, about 47 months, about 46 months, about 45 months, about 44 months, about 43 months, about 42 months, about 41 months, about 40 months, about 39 months, about 38 months, about 37 months, about 36 months, about 35 months, about 34 months, about 33 months, about 32 months, about 31 months, about 30 months, about 29 months, about 28 months, about 27 months, about 26 months, about 25 months, about 24 months, about 23 months, about 22 months, about 21 months, about 20 months, about 19 months, about 18 months, about 17 months, about 16 months, about 15 months, about 14 months, about 13 months, about 12 months, about 11 months, about 10 months, about 9 months, about 8 months, about 7 months, about 6 months, about 5 months, about 4 months, about 3 months, about 2 months, or about 1 month. In various examples, the pharmaceutical formulation may be stable and suitable for administration after storage for more than 3 months, more than 6 months, more than 12 months, more than 18 months, more than 24 months, more than 36 months, more than 48 months, more than 60 months, or more than 70 months. For example, the pharmaceutical formulation may be stable and suitable for administration after storage for 3 months to 72 months, 3 months to 6 months, 3 months to 24 months, 3 months to 36 months, 3 months to 48 months, 3 months to 60 months, 6 months to 12 months, 6 months to 24 months, 6 months to 48 months, 6 months to 36 months, 6 months to 48 months, 6 months to 60 months, 6 months to 72 months, 12 months to 24 months, 12 months to 36 months, 12 months to 48 months, 12 months to 60 months, 12 months 72 months, 24 months to 36 months, 24 months to 48 months, 24 months to 60 months, 24 months to 72 months, 36 months to 48 months, 36 months to 60 months, 36 months to 72 months, 48 months to 60 months, 48 months to 72 months, or 60 months to 72 months at 25° C. to 40° C. and 60% to 75% relative humidity. In some examples, the pharmaceutical formulation may be stable and suitable for administration after storage for more than 72 months.

The pharmaceutical formulation may be stored at about 25° C., about 30° C., about 35° C., about 40° C. or any temperature therebetween. The packaged pharmaceutical formulation may be stored at a relative humidity of about 60%, about 65%, about 70%, about 75%, or any RH therebetween.

In some embodiments, the pharmaceutical formulation may have a pH of ≤5.9, ≤5.8, ≤5.7, or ≤5.6 after the pharmaceutical formulation has been stored for up to 3 months to up to 72 months at 25° C. to 40° C. and 60% to 75% relative humidity. In some examples, the pharmaceutical formulation may have a pH of 5.6 to 5.9, 5.6 to 5.8, or 5.7 to 5.9 after the pharmaceutical formulation has been stored for up to 6 months to up to 72 months at 25° C. to 40° C. and 60% to 75% relative humidity. In some examples, the pharmaceutical formulation may have a pH of 5.6, 5.7, 5.8, or 5.9 after the pharmaceutical formulation has been stored for up to 3 months to up to 72 months at 25° C. to 40° C. and 60% to 75% relative humidity.

In an example, the pharmaceutical formulation has a pH of 5.6 to 5.9 after the pharmaceutical formulation has been stored for up to about 72 months, about 60 months, about 48 months, about 36 months, about 24 months, about 18 months, about 12 months, about 6 months, or about 3 months at 25° C. and 60% RH. In another example, the pharmaceutical formulation has a pH of 5.6 to 5.9 after the pharmaceutical formulation has been stored for up to about 24 months, about 18 months, about 15 months, about 12 months, or about 6 months at 30° C. and 65% RH. In yet another example, the pharmaceutical formulation has a pH of 5.6 to 5.9 after the pharmaceutical formulation has been stored for up to about 6 months or about 3 months at 40° C. and 75% RH.

In some embodiments, ≤ about 0.18% w/w, ≤ about 0.17% w/w, ≤ about 0.16% w/w, ≤ about 0.15% w/w, ≤ about 0.14% w/w, ≤ about 0.13% w/w, ≤ about 0.12% w/w, ≤ about 0.11% w/w, ≤ about 0.10% w/w, ≤ about 0.09% w/w, ≤ about 0.08% w/w, ≤ about 0.07% w/w, ≤ about 0.06% w/w, or ≤ about 0.05% w/w gamma-butyrolactone is present in the pharmaceutical formulation after the pharmaceutical formulation has been stored for up to 3 months to up to 72 months at 25° C. to 40° C. and 60% to 75% relative humidity. In other embodiments, about 0.18% w/w to about 0.15% w/w, about 0.17% w/w to about 0.14% w/w, about 0.16% w/w to about 0.13% w/w, about 0.15% w/w to about 0.12% w/w, about 0.14% w/w to about 0.11% w/w, about 0.13% w/w to about 0.10% w/w, about 0.12% w/w to about 0.9% w/w, about 0.11% w/w to about 0.08% w/w, about 0.10% w/w to about 0.7% w/w, about 0.09% w/w to about 0.06% w/w, about 0.08% w/w to about 0.05% w/w, or ≤ about 0.05% w/w gamma-butyrolactone is present in the pharmaceutical formulation after the pharmaceutical formulation has been stored for up to about 3 months up to 72 months at 25° C. to 40° C. and 60% to 75% relative humidity.

In an example, ≤0.18% w/w, ≤0.13% w/w, ≤0.11% w/w, ≤0.08% w/w, ≤0.07% w/w, or ≤0.05% w/w gamma-butyrolactone is present in the pharmaceutical formulation after the pharmaceutical formulation has been stored for up to about 72 months, about 60 months, about 48 months, about 36 months, about 24 months, about 18 months, about 12 months, about 6 months, or about 3 months at 25° C. and 60% RH. In another example, ≤0.18% w/w, ≤0.13% w/w, ≤0.11% w/w, ≤0.08% w/w, ≤0.07% w/w, or ≤0.05% gamma-butyrolactone is present in the pharmaceutical formulation after the pharmaceutical formulation has been stored for up to about 24 months, about 18 months, about 15 months, about 12 months, or about 6 months at 30° C. and 65% RH. In yet another example, ≤0.18% w/w, ≤0.13% w/w, ≤0.11% w/w, ≤0.08% w/w, ≤0.07% w/w, or ≤0.05% gamma-butyrolactone is present in the package after the pharmaceutical formulation has been stored for up to about 6 months or about 3 months at 40° C. and 75% RH.

In some embodiments, the amount of gamma-hydroxybutyrate in the pharmaceutical formulation after the pharmaceutical formulation has been stored for up to 3 months to up to 72 months at 25° C. to 40° C. and 60% to 75% relative humidity is 90.0% to 110.0%, 90.0% to 100.0%, 95.0% to 105%, or about 100.0% to 110.0% of the amount of gamma-hydroxybutyrate in the pharmaceutical formulation before storage. In some embodiments, the amount of gamma-hydroxybutyrate in the pharmaceutical formulation after the pharmaceutical formulation has been stored for up to 3 months to up to 72 months at 25° C. to 40° C. and 60% to 75% relative humidity is about 90%, about 91%, 92%, 93%, 94%, 95%, 96%, 97%, 98%, 99%, 100%, 101%, 102%, 103%, 104%, 105%, 106%, 107%, 108%, 109%, or 110% of the amount of gamma-hydroxybutyrate in the pharmaceutical formulation before storage.

In some examples, the amount of gamma-hydroxybutyrate in the pharmaceutical formulation after the pharmaceutical formulation has been stored for up to about 72 months, about 60 months, about 48 months, about 36 months, about 24 months, about 18 months, about 12 months, about 6 months, or about 3 months at 25° C. and 60% RH is 99.0% to 105.0% of the amount of gamma-hydroxybutyrate in the pharmaceutical formulation before storage. In another example, the amount of gamma-hydroxybutyrate in the pharmaceutical formulation after the pharmaceutical formulation has been stored for up to about 24 months, about 18 months, about 15 months, about 12 months, or about 6 months at 30° C. and 65% RH is 99.0% to 105.0% of the amount of gamma-hydroxybutyrate in the pharmaceutical formulation before storage. In another example, the amount of gamma-hydroxybutyrate in the pharmaceutical formulation after the pharmaceutical formulation has been stored for up to about 6 months or about 3 months at 40° C. and 75% RH is 99.0% to 105.0% of the amount of gamma-hydroxybutyrate in the pharmaceutical formulation before storage.

In some embodiments, the water content in the pharmaceutical formulation is ≤4.0% w/w, ≤3.0% w/w, ≤2.0% w/w, ≤1.0% w/w, ≤0.9% w/w, ≤0.8% w/w, ≤0.7% w/w, ≤0.6% w/w, ≤0.5% w/w, or ≤0.4% w/w after the pharmaceutical formulation has been stored for up to about 3 months to up to about 72 months at 25° C. to 40° C. and 60% to 75% relative humidity. Stated differently, the water content in the pharmaceutical formulation may be about 2.0% to about 4.0% w/w, about 1.0% w/w to about 3.0% w/w, about 0.5% w/w to about 2.0% w/w, about 0.5% w/w to about 1.0% w/w, about 0.4% w/w to about 0.9% w/w, about 0.3% w/w to about 0.8% w/w, about 0.2% w/w to about 0.7% w/w, or about 0.1% w/w to about 0.6% w/w after the pharmaceutical formulation has been stored for up to about 3 months to up to about 72 months at 25° C. to 40° C. and 60% to 75% relative humidity.

For example, the water content in the pharmaceutical formulation is ≤2.0% w/w, ≤1.0% w/w, ≤0.9% w/w, ≤0.8% w/w, ≤0.7% w/w, ≤0.6% w/w, ≤0.5% w/w, or ≤0.4% w/w after the pharmaceutical formulation has been stored for up to about 72 months, about 60 months, about 48 months, about 36 months, about 24 months, about 18 months, about 12 months, about 6 months, or about 3 months at 25° C. and 60% RH. In an example, the water content in the pharmaceutical formulation is ≤2.0% w/w, ≤1.0% w/w, ≤0.9% w/w, ≤0.8% w/w, ≤0.7% w/w, ≤0.6% w/w, ≤0.5% w/w, or ≤0.4% w/w after the pharmaceutical formulation has been stored for up to about 24 months, about 18 months, about 15 months, about 12 months, or about 6 months at 30° C. and 65% RH. In another example, the water content in the pharmaceutical formulation is ≤2.0% w/w, ≤1.0% w/w, ≤0.9% w/w, ≤0.8% w/w, ≤0.7% w/w, ≤0.6% w/w, ≤0.5% w/w, or ≤0.4% w/w after the pharmaceutical formulation has been stored for up to about 6 months or about 3 months at 40° C. and 75% RH.

### Sub-Embodiments

As mentioned in the definitions section of this document, each of the sub-embodiments can be used to further characterize and limit each of the foregoing principal embodiments. In addition, more than one of the following sub-embodiments can be combined and used to further characterize and limit each of the foregoing principal embodiments, in any manner that is mathematically and physically possible.

In various sub-embodiments, the composition is defined based on its dissolution stability. Thus, in some subembodiments, after a two-month 40° C./75% relative humidity storage period the composition exhibits a lag time that is less than 70, 60, or 50 minutes different than the lag time exhibited at the beginning of the storage period, wherein the lag time is determined from testing in a dissolution apparatus 2 according to USP 38<711> in 900 mL of 0.1N hydrochloric acid at a temperature of 37° C. and a paddle speed of 75 rpm.

In other subembodiments, the quantity of gamma-hydroxybutyrate dissolved after a two-month 40° C./75% relative humidity storage period is less than 10% different than the quantity of gamma-hydroxybutyrate dissolved before the storage period when tested in a dissolution apparatus 2 according to USP 38<711> in 900 mL of 0.1N hydrochloric acid at a temperature of 37° C. and a paddle speed of 75 rpm at 4, 6 or 8 consecutive hourly time points.

In other subembodiments the packaged composition is defined based on its chemical stability. Thus, in another subembodiment, the package prevents no more than 0.4% of the gamma-hydroxybutyrate from converting to gamma-butyrolactone (GBL) when stored two months at 40° C. and 75% relative humidity.

### Packaging

In one particular subembodiment, applicable to any of the principal embodiments, the composition is housed inside a package's interior volume. The atmosphere inside the interior volume is preferably defined in terms of its humidity or its humidity over time. In one subembodiment, the atmosphere inside the interior volume has a relative humidity in a range of from 29% to 54% and the package maintains the relative humidity in the range for a period of at least 2 months when stored at 40° C. and 75% relative humidity. In another subembodiment, the interior volume has a relative humidity of greater than 29% at 1 week and less than 54% at 2 months when stored at 40° C. and 75% relative humidity. In another subembodiment, the interior volume has a relative humidity of greater than 29% and less than 44% at one week and less than 54% at 2 months when stored at 40° C. and 75% relative humidity. In yet another subembodiment, the interior volume has a relative humidity of from 35 to 39% after one week and from 39 to 48% after 2 months when stored at 40° C. and 75% relative humidity.

In one subembodiment, the dissolution profile is unstable, and the packaging is not suitable, if:
after one week at 40° C./75% RH, RH is below 29%; or
before 2 two months at 40° C./75% RH, RH is higher than 54%

The package can further be defined based on its water vapor transmission rate. In various subembodiments, the package has a water vapor transmission rate of less than 7, 3.5, or 1 mg/day/liter when measured according to USP 38<671>. Particular packaging materials include an aluminum foil pouch or sachet or stick-pack, as well as modified HDPE bottles with decreased water permeability such as the H2OO2™ bottle manufactured by LOG Pharma Packaging (Israel).

When an aluminum foil pouch or sachet or stick-pack is used, further subembodiments can be defined based on the thickness of the aluminum film. This, in various other subembodiments, the aluminum film used in the packaging has a thickness equal to or greater than 6 μm, 9 μm, or 12 μm.

The modified release formulation of gamma-hydroxybutyrate is typically supplied in sachets or stick-packs comprising a particulate formulation. The sachets or stick-packs are typically available in several different doses, comprising gamma-hydroxybutyrate in amounts equivalents to 0.5 g, 1.0 g, 1.5 g, 3.0 g, 4.5 g, 6.0 g, 7.5 g, 9.0 g, 10.5 g and/or 12 g of sodium oxybate. Depending on the dose required, one or more of these sachets or stick-packs can be opened, and its contents mixed with tap or drinking water to provide the nightly dose of gamma-hydroxybutyrate.

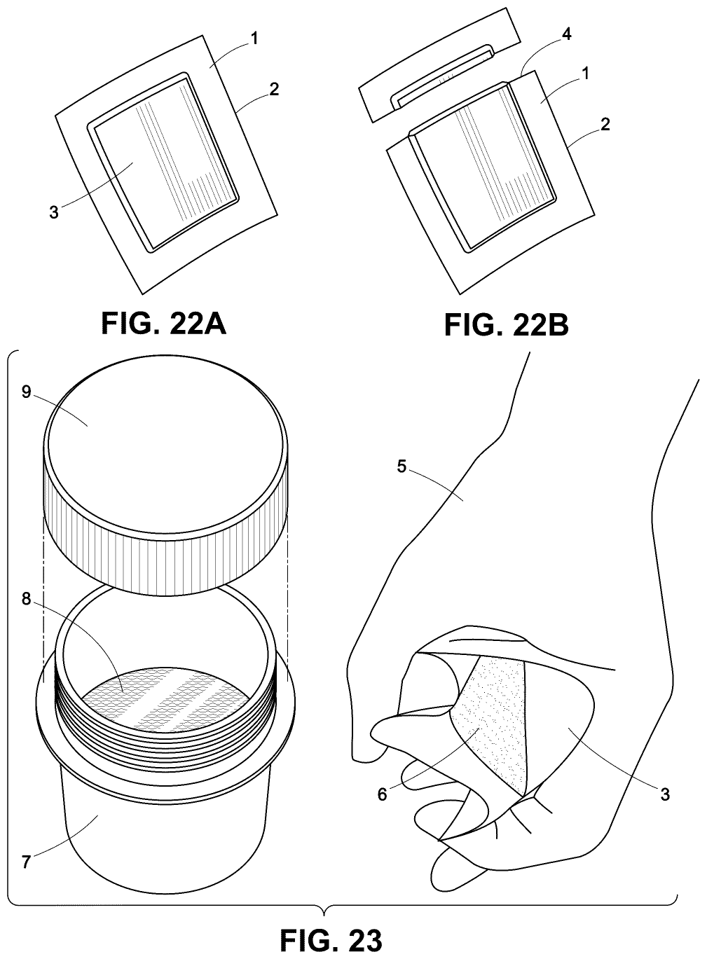

Turning to FIGS. 22-24, one can see various embodiments of exemplary packaging of the present invention and uses of the packaging. FIGS. 22A and 22B depict a planar view of sachet-type packaging for use in the present invention. The packaging comprises two flat sheets of equal dimension (1) sealed to one another around their periphery (2) in FIG. 22A to define a hollow interior (3) in which the drug product is packaged. In FIG. 22B the packaging is cut across one end (4) so that the drug product can be dispensed.

FIG. 23 depicts the left hand of an individual (5) holding open the sachet depicted in FIG. 22B, with the drug contents (6) in the hollow interior (3) exposed and ready to be poured into a cup (7) of water (8) which is also depicted. After drug contents (6) are poured into cup (7) and mixed with water (8), cap (9) is screwed onto the top of cup (7) so that the contents can be shaken into a homogenous suspension.

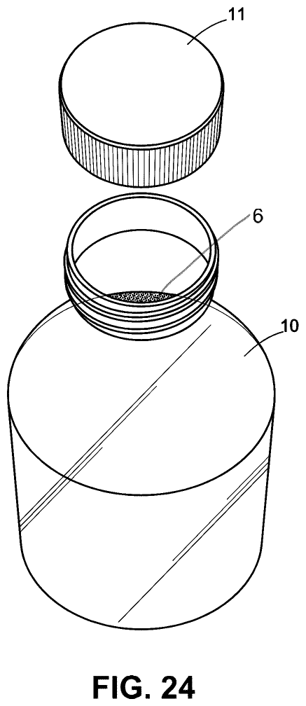

FIG. 24 depicts an alternative type of packaging for the drug product of the present invention. The packaging is a bottle (10) constructed of moisture resistant material, and has a screw lid cap (11) removed thereby exposing the drug product (6) inside the bottle.

### Composition Sub-Embodiments

The gamma-hydroxybutyrate composition of the present invention can be provided in any dosage form that is suitable for oral administration, including tablets, capsules, liquids, orally dissolving tablets, and the like, but they are typically provided as dry particulate formulations (i.e. granules, powders, coated particles, microparticles, pellets, microspheres, etc.), in a sachet or other suitable discreet packaging units. A preferred particulate formulation will be mixed with water shortly before administration, preferably 50 mL.

In various subembodiments, when the composition is a particulate formulation, the formulation will include excipients to improve the viscosity and the pourability of the mixture of the particulate formulation with water. As such, the particulate formulation comprises, besides the immediate release and modified release particles of gamma-hydroxybutyrate, one or more suspending or viscosifying agents or lubricants.

Particular suspending or viscosifying agents are chosen from the group consisting of xanthan gum, medium viscosity sodium carboxymethyl cellulose, mixtures of microcrystalline cellulose and sodium carboxymethyl cellulose, mixtures of microcrystalline cellulose and guar gum, medium viscosity hydroxyethyl cellulose, agar, sodium alginate, mixtures of sodium alginate and calcium alginate, gellan gum, carrageenan gum grade iota, kappa or lambda, and medium viscosity hydroxypropylmethyl cellulose.

Medium viscosity sodium carboxymethyl cellulose corresponds to grade of sodium carboxymethyl cellulose whose viscosity, for a 2% solution in water at 25° C., is greater than 200 mPa's and lower than 3100 mPa·s.

Medium viscosity hydroxyethyl cellulose corresponds to a grade of hydroxyethyl cellulose whose viscosity, for a 2% solution in water at 25° C., is greater than 250 mPa·s and lower than 6500 mPa·s. Medium viscosity hydroxypropylmethyl cellulose corresponds to a grade of hydroxypropylmethyl cellulose whose viscosity, for a 2% solution in water at 20° C., is greater than 80 mPa·s, and lower than 3800 mPa·s.

Particular suspending or viscosifying agents are xanthan gum, especially Xantural 75™ from Kelco, hydroxyethylcellulose, especially Natrosol 250M™ from Ashland, Kappa carrageenan gum, especially Gelcarin PH812™ from FMC Biopolymer, and lambda carrageenan gum, especially Viscarin PH209™ from FMC Biopolymer.

In a particular embodiment, the gamma-hydroxybutyrate formulation comprises from 1 to 15% of viscosifying or suspending agents, typically from 2 to 10%, more typically from 2 to 5%, and most preferably from 2 to 3% of the formulation.

In a particular embodiment, the formulation of gamma-hydroxybutyrate is in the form of a powder that is intended to be dispersed in water prior to administration and further comprises from 1 to 15% of a suspending or viscosifying agent selected from a mixture of xanthan gum, carrageenan gum and hydroxyethylcellulose or xanthan gum and carrageenan gum.

In a particular embodiment, the formulation of gamma-hydroxybutyrate is in the form of a powder that is intended to be dispersed in water prior to administration and further comprises: from 1.2 to 15% of an acidifying agent selected from malic acid and tartaric acid; and from 1 to 15% of a suspending or viscosifying agent selected from a mixture of xanthan gum, carrageenan gum and hydroxyethylcellulose or xanthan gum and carrageenan gum.

In a most preferred embodiment, the formulation of gamma-hydroxybutyrate comprises about 1% of lambda carrageenan gum or Viscarin PH209™, about 1% of medium viscosity grade of hydroxyethyl cellulose or Natrosol 250M™, and about 0.7% of xanthan gum or Xantural 75™. For a 4.5 g dose unit, these percentages will typically equate to about 50 mg xanthan gum (Xantural 75™), about 75 mg carrageenan gum (Viscarin PH209™), and about 75 mg hydroxyethylcellulose (Natrasol 250M™).

Alternative packages of viscosifying or suspending agents, for a 4.5 g dose, include about 50 mg xanthan gum (Xantural 75™) and about 100 mg carrageenan gum (Gelcarin PH812™), or about 50 mg xanthan gum (Xantural 75™), about 75 mg hydroxyethylcellulose (Natrasol 250M™), and about 75 mg carrageenan gum (Viscarin PH109™).

In a particular embodiment, the formulation of gamma-hydroxybutyrate further comprises a lubricant or a glidant, besides the immediate release and modified release particles of gamma-hydroxybutyrate. Particular lubricants and glidants are chosen from the group consisting of salts of stearic acid, in particular magnesium stearate, calcium stearate or zinc stearate, esters of stearic acid, in particular glyceryl monostearate or glyceryl palmitostearate, stearic acid, glycerol behenate, sodium stearyl fumarate, talc, and colloidal silicon dioxide. The preferred lubricant or glidant is magnesium stearate.

The lubricant or glidant can be used in the particulate formulation in an amount of from 0.1 to 5%. The preferred amount is about 0.5%. Most preferably, the modified release formulation of gamma-hydroxybutyrate comprises about 0.5% of magnesium stearate.

A particular formulation of gamma-hydroxybutyrate further comprises an acidifying agent. The acidifying agent helps to ensure that the dissolution profile of the formulation in 0.1N HCl will remain substantially unchanged for at least 15 minutes after mixing, even 30 minutes after mixing, which is approximately the maximum length of time a patient might require before consuming the dose after mixing the formulation with tap water.

In one particular subembodiment the formulation is a powder, and further comprising an acidifying agent and a suspending or viscosifying agent, typically in the weight percentages recited herein.

The particular acidifying agents are chosen from the group consisting of malic acid, citric acid, tartaric acid, adipic acid, boric acid, maleic acid, phosphoric acid, ascorbic acid, oleic acid, capric acid, caprylic acid, and benzoic acid. In a particular embodiment, the acidifying agent is typically present in the formulation from 1.2 to 15%, from 1.2 to 10%, or from 1.2 to 5%. Preferred acidifying agents are tartaric acid and malic acid, with malic acid being most preferred.

When tartaric acid is employed, it is typically employed in an amount of from 1 to 10%, from 2.5 to 7.5%, or about 5%. In a most preferred embodiment, the amount of malic acid in the modified release formulation of gamma-hydroxybutyrate is from 1.2 to 15%, typically from 1.2 to 10%, typically from 1.2 to 5%, and most preferably 1.6% or 3.2%.

In a most a particular embodiment, the amount of malic acid in the modified release formulation of gamma-hydroxybutyrate is about 1.6%.

The molar ratio of gamma-hydroxybutyrate in the immediate release and modified release components typically ranges from 0.11:1 to 1.86:1, from 0.17:1 to 1.5:1, from 0.25:1 to 1.22:1, from 0.33:1 to 1.22:1, from 0.42:1 to 1.22:1, from 0.53:1 to 1.22:1, from 0.66:1 to 1.22:1, from 0.66:1 to 1.5:1, from 0.8:1 to 1.22:1, and preferably is about 1:1. The molar percentage of gamma-hydroxybutyrate in the immediate release component relative to the total of gamma-hydroxybutyrate in the formulation typically ranges from 10% to 65%, from 15 to 60%, from 20 to 55%, from 25 to 55%, from 30 to 55%, from 35 to 55%, from 40 to 55%, from 40 to 60%, or from 45 to 55%, preferably from 40% to 60%. In a particular embodiment, the molar percentage of the gamma-hydroxybutyrate in the immediate release component relative to the total of gamma-hydroxybutyrate in the formulation is about 50%. The molar percentage of gamma-hydroxybutyrate in the modified release component relative to the total of gamma-hydroxybutyrate in the formulation typically ranges from 90% to 35%, from 85 to 40%, from 80 to 45%, from 75 to 45%, from 70 to 45%, from 65 to 45%, from 60 to 45%, from 60 to 40%, or from 55 to 45%, preferably from 60% to 40%. In a particular embodiment, the molar ratio of the gamma-hydroxybutyrate in the modified release component relative to the total of gamma-hydroxybutyrate in the formulation is about 50%. The weight percentage of the IR microparticles relative to the total weight of IR microparticles and MR microparticles, typically ranges from 7.2% to 58.2%, from 11.0% to 52.9%, from 14.9% to 47.8%, from 18.9% to 47.8%, from 23.1% to 47.8%, from 27.4% to 47.8%, from 31.8% to 47.8%, from 31.8% to 52.9%, or from 36.4% to 47.8%. In other embodiments, the weight percentage of the IR microparticles relative to the total weight of IR microparticles and MR microparticles typically ranges from 5.9% to 63.2%, from 9.1% to 58.1%, from 12.4% to 53.1%, from 19.9% to 53.1%, from 19.6% to 53.1%, from 23.4% to 53.1%, from 27.4% to 53.1% from 27.4% to 58.1%, preferably from 31,7% to 53.1%.

In a particular embodiment, the finished formulation comprises 50% of its sodium oxybate content in immediate-release particles consisting of 80.75% w/w of sodium oxybate, 4.25% w/w of Povidone K30 and 15% of microcrystalline cellulose spheres with a volume mean diameter of about 95 microns to 450 microns and 50% of its sodium oxybate content in modified release particles consisting of 10.5% w/w of microcrystalline cellulose spheres with a volume mean diameter of about 95 microns to about 450 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of Eudragit™ L100-55 (methacrylic acid and ethyl acrylate copolymer NF) and 8% of Eudragit™ S100 (methacrylic acid and methyl methacrylate copolymer (1:2) NF).

In a particular embodiment, the finished formulation comprises 50% of its sodium oxybate content in immediate-release particles consisting of 80.75% w/w of sodium oxybate, 4.25% w/w of Povidone K30 and 15% of microcrystalline cellulose spheres with a volume mean diameter of about 95 microns to 170 microns and 50% of its sodium oxybate content in modified release particles consisting of 10.5% w/w of microcrystalline cellulose spheres with a volume mean diameter of about 95 microns to about 170 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of Eudragit™ L100-55 (methacrylic acid and ethyl acrylate copolymer NF) and 8% of Eudragit™ S100 (methacrylic acid and methyl methacrylate copolymer (1:2) NF).

In a particular embodiment, the finished formulation comprises 50% of its sodium oxybate content in immediate-release particles consisting of 80.75% w/w of sodium oxybate, 4.25% w/w of Povidone K30 and 15% of microcrystalline cellulose spheres with a volume mean diameter of about 95 microns to about 450 microns and 50% of its sodium oxybate content in modified release particles consisting of 11.3% w/w of microcrystalline cellulose spheres with a volume mean diameter of about 95 microns to about 450 microns, layered with 60.5% w/w of sodium oxybate mixed with 3.2% w/w of Povidone™ K30 and finally coated with a coating composition consisting of 15% w/w of hydrogenated vegetable oil (Lubritab™ or equivalent), 0.75% of Eudragit™ L100-55 (methacrylic acid and ethyl acrylate copolymer NF) and 9.25% of Eudragit™ S100 (methacrylic acid and methyl methacrylate copolymer (1:2) NF).

In a particular embodiment, the finished formulation comprises 50% of its sodium oxybate content in immediate-release particles consisting of 80.75% w/w of sodium oxybate, 4.25% w/w of Povidone™ K30 and 15% of microcrystalline cellulose spheres with a volume mean diameter of about 95 microns to about 170 microns and 50% of its sodium oxybate content in modified release particles consisting of 11.3% w/w of microcrystalline cellulose spheres with a volume mean diameter of about 95 microns to about 170 microns, layered with 60.5% w/w of sodium oxybate mixed with 3.2% w/w of Povidone™ K30 and finally coated with a coating composition consisting of 15% w/w of hydrogenated vegetable oil (Lubritab™ or equivalent), 0.75% of Eudragit™ L100-55 (methacrylic acid and ethyl acrylate copolymer NF) and 9.25% of Eudragit™ S100 (methacrylic acid and methyl methacrylate copolymer (1:2) NF).

In a particular embodiment, the finished formulation comprises 50% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 450 microns and 50% of its gamma-hydroxybutyrate content in modified release particles consisting of 10.5% w/w of microcrystalline cellulose spheres with a volume mean diameter of about 95 microns to about 450 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of Eudragit™ L100-55 (methacrylic acid and ethyl acrylate copolymer NF) and 8% of Eudragit™ S100 (methacrylic acid and methyl methacrylate copolymer (1:2) NF).

In a particular embodiment, the finished formulation comprises 50% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 170 microns and 50% of its gamma-hydroxybutyrate content in modified release particles consisting of 10.5% w/w of microcrystalline cellulose spheres with a volume mean diameter of about 95 microns to about 170 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of Eudragit™ L100-55 (methacrylic acid and ethyl acrylate copolymer NF) and 8% of Eudragit™ S100 (methacrylic acid and methyl methacrylate copolymer (1:2) NF).

In a particular embodiment, the finished formulation comprises 16.7% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 450 microns, 16.7% of its gamma-hydroxybutyrate content in immediate-release particles consisting of 80.75% w/w of magnesium salt of gamma-hydroxybutyric acid, 4.25% w/w of Povidone K30 and 15% of microcrystalline cellulose spheres with a volume mean diameter of about 95 microns to about 450 microns, 16.7% of its gamma-hydroxybutyrate content in immediate-release particles consisting of 80.75% w/w of calcium salt of gamma-hydroxybutyric acid, 4.25% w/w of Povidone K30 and 15% of microcrystalline cellulose spheres with a volume mean diameter of about 95 microns to about 450 microns and 50% of its gamma-hydroxybutyrate content in modified release particles consisting of 10.5% w/w of microcrystalline cellulose spheres with a volume mean diameter of about 95 microns to about 450 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of Eudragit™ L100-55 (methacrylic acid and ethyl acrylate copolymer NF) and 8% of Eudragit™ S100 (methacrylic acid and methyl methacrylate copolymer (1:2) NF).

In a particular embodiment, the finished formulation comprises 16.7% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 170 microns, 16.7% of its gamma-hydroxybutyrate content in immediate-release particles consisting of 80.75% w/w of magnesium salt of gamma-hydroxybutyric acid, 4.25% w/w of Povidone K30 and 15% of microcrystalline cellulose spheres with a volume mean diameter of about 95 microns to about 170 microns, 16.7% of its gamma-hydroxybutyrate content in immediate-release particles consisting of 80.75% w/w of calcium salt of gamma-hydroxybutyric acid, 4.25% w/w of Povidone K30 and 15% of microcrystalline cellulose spheres with a volume mean diameter of about 95 microns to about 170 microns and 50% of its gamma-hydroxybutyrate content in modified release particles consisting of 10.5% w/w of microcrystalline cellulose spheres with a volume mean diameter of about 95 microns to about 170 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of Eudragit™ L100-55 (methacrylic acid and ethyl acrylate copolymer NF) and 8% of Eudragit™ S100 (methacrylic acid and methyl methacrylate copolymer (1:2) NF).

In a particular embodiment, the finished formulation comprises 50% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 450 microns and 50% of its gamma-hydroxybutyrate content in modified release particles consisting of 10.5% w/w of microcrystalline cellulose spheres with a volume mean diameter of about 95 microns to about 450 microns, layered with 56.5% w/w of calcium salt of gamma-hydroxybutyric acid mixed with 3% w/w of Povidone™ K30 and finally coated with a coating composition consisting of 18% w/w of hydrogenated vegetable oil (Lubritab™ or equivalent), 4% of Eudragit™ L100-55 (methacrylic acid and ethyl acrylate copolymer NF) and 8% of Eudragit™ S100 (methacrylic acid and methyl methacrylate copolymer (1:2) NF).

In a particular embodiment, the finished formulation comprises 50% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 170 microns and 50% of its gamma-hydroxybutyrate content in modified release particles consisting of 10.5% w/w of microcrystalline cellulose spheres with a volume mean diameter of about 95 microns to about 170 microns, layered with 56.5% w/w of calcium salt of gamma-hydroxybutyric acid mixed with 3% w/w of Povidone™ K30 and finally coated with a coating composition consisting of 18% w/w of hydrogenated vegetable oil (Lubritab™ or equivalent), 4% of Eudragit™ L100-55 (methacrylic acid and ethyl acrylate copolymer NF) and 8% of Eudragit™ S100 (methacrylic acid and methyl methacrylate copolymer (1:2) NF).

### Other Characteristics of Immediate Release Component

The immediate release component of the formulation can take any form capable of achieving an immediate release of the gamma-hydroxybutyrate when ingested. For example, when the formulation is a particulate formulation, the formulation can include unmodified “raw” gamma-hydroxybutyrate, rapidly dissolving gamma-hydroxybutyrate granules, particles or microparticles comprised of a core covered by a gamma-hydroxybutyrate loaded layer containing a binder such as povidone.

The IR granules or particles of gamma-hydroxybutyrate can be made using any manufacturing process suitable to produce the required particles, including:
agglomeration of the gamma-hydroxybutyrate sprayed typically in the molten state, such as the Glatt ProCell™ technique,
extrusion and spheronization of the gamma-hydroxybutyrate, optionally with one or more physiologically acceptable excipients,
wet granulation of the gamma-hydroxybutyrate, optionally with one or more physiologically acceptable excipients,
compacting of the gamma-hydroxybutyrate, optionally with one or more physiologically acceptable excipients,
granulation and spheronization of the gamma-hydroxybutyrate, optionally with one or more physiologically acceptable excipients, the spheronization being carried out for example in a fluidized bed apparatus equipped with a rotor, in particular using the Glatt CPS™ technique,
spraying of the gamma-hydroxybutyrate, optionally with one or more physiologically acceptable excipients, for example in a fluidized bed type apparatus equipped with zig-zag filter, in particular using the Glatt MicroPx™ technique, or
spraying, for example in a fluidized bed apparatus optionally equipped with a partition tube or Wurster tube, the gamma-hydroxybutyrate, optionally with one or more physiologically acceptable excipients, in dispersion or in solution in an aqueous or organic solvent on a core.

Typically, the immediate release component of the formulation is in the form of microparticles comprising the immediate release gamma-hydroxybutyrate and optional pharmaceutically acceptable excipients. In a particular embodiment, the immediate release microparticles of gamma-hydroxybutyrate have a volume mean diameter D(4,3) of from 10 to 1000 microns, typically from 95 to 600 microns, more typically from 150 to 400 microns. Most preferably their volume mean diameter is about 270 microns.

The preferred immediate release particles of gamma-hydroxybutyrate of the present invention comprise a core and a layer deposited on the core that contains the gamma-hydroxybutyrate. The core can be any particle chosen from the group consisting of:
crystals or spheres of lactose, sucrose (such as Compressuc™ PS from Tereos), microcrystalline cellulose (such as Avicel™ from FMC Biopolymer, Cellet™ from Pharmatrans or Celphere™ from Asahi Kasei), sodium chloride, calcium carbonate (such as Omyapure™ 35 from Omya), sodium hydrogen carbonate, dicalcium phosphate (such as Dicafos™ AC 92-12 from Budenheim) or tricalcium phosphate (such as Tricafos™ SC93-15 from Budenheim);
composite spheres or granules, for example sugar spheres comprising sucrose and starch (such as Suglets™ from NP Pharm), spheres of calcium carbonate and starch (such as Destab™ 90 S Ultra 250 from Particle Dynamics) or spheres of calcium carbonate and maltodextrin (such as Hubercal™ CCG4100 from Huber).

The core can also comprise other particles of pharmaceutically acceptable excipients such as particles of hydroxypropyl cellulose (such as Klucel™ from Aqualon Hercules), guar gum particles (such as Grinsted™ Guar from Danisco), xanthan particles (such as Xantural™ 180 from CP Kelco).

According to a particular embodiment of the invention, the cores are sugar spheres or microcrystalline cellulose spheres, such as Cellets™90, Cellets™100 or Cellets™127 marketed by Pharmatrans, or also Celphere™ CP 203, Celphere™ CP305, Celphere™ SCP 100. Typically the core is a microcrystalline cellulose sphere. Most preferably the core is a Cellets™127 from Pharmatrans.

The core typically has a mean volume diameter of about 95 to about 450 microns, more typically about 95 to about 170 microns, most preferably about 140 microns.

The layer deposited onto the core comprises the immediate release gamma-hydroxybutyrate. Typically the layer also comprises a binder, which can be chosen from the group consisting of:
low molecular weight hydroxypropyl cellulose (such as Klucel™μF from Aqualon-Hercules), low molecular weight hydroxypropyl methylcellulose (or hypromellose) (such as Methocel™E3 or E5 from Dow), or low molecular weight methylcellulose (such as Methocel™A15 from Dow);
low molecular weight polyvinyl pyrrolidone (or povidone) (such as Plasdone™ K29/32 from ISP or Kollidon™M30 from BASF), vinyl pyrrolidone and vinyl acetate copolymer (or copovidone) (such as Plasdone™: S630 from ISP or Kollidon™VA 64 from BASF);
dextrose, pregelatinized starch, maltodextrin; and mixtures thereof.

Low molecular weight hydroxypropyl cellulose corresponds to grades of hydroxypropyl cellulose having a molecular weight of less than 800,000 g/mol, typically less than or equal to 400,000 g/mol, and in particular less than or equal to 100,000 g/mol. Low molecular weight hydroxypropyl methylcellulose (or hypromellose) corresponds to grades of hydroxypropyl methylcellulose the solution viscosity of which, for a 2% solution in water and at 20° C., is less than or equal to 1,000 mPa·s, typically less than or equal to 100 mPa's and in particular less than or equal to 15 mPa·s. Low molecular weight polyvinyl pyrrolidone (or povidone) corresponds to grades of polyvinyl pyrrolidone having a molecular weight of less than or equal to 1,000,000 g/mol, typically less than or equal to 800,000 g/mol, and in particular less than or equal to 100,000 g/mol.

Typically, the binding agent is chosen from low molecular weight polyvinylpyrrolidone or povidone (for example, Plasdone™ K29/32 from ISP), low molecular weight hydroxypropyl cellulose (for example, Klucel™EF from Aqualon-Hercules), low molecular weight hydroxypropyl methylcellulose or hypromellose (for example, Methocel™E3 or E5 from Dow) and mixtures thereof.

The preferred binder is povidone K30 or K29/32, especially Plasdone™ K29/32 from ISP. The binder can be present in an amount of 0 to 80%, 0 to 70%, 0 to 60%, 0 to 50%, 0 to 40%, 0 to 30%, 0 to 25%, 0 to 20%, 0 to 15%, 0 to 10%, or from 1 to 9%, most preferably 5% of binder based on the total weight of the immediate release coating.

The preferred amount of binder is 5% of binder over the total mass of gamma-hydroxybutyrate and binder.

The layer deposited on the core can represent at least 10% by weight, and even greater than 20, 25, 30, 35, 40, 45, 50, 55, 60, 65, 70, 75, 80, 85 or 90% by weight of the total weight of the immediate release particle of gamma-hydroxybutyrate. Most preferably, the layer deposited on the core represents about 85% of the weight of the immediate release particle of gamma-hydroxybutyrate.

According to a particular embodiment, the immediate-release particles comprise 80.75% w/w of gamma-hydroxybutyrate, 4.25% w/w of Povidone K30 and 15% of microcrystalline cellulose spheres.

According to a particular embodiment, the immediate-release particles comprise 80.75% w/w of gamma-hydroxybutyrate, 4.25% w/w of Povidone K30 and 15% of microcrystalline cellulose spheres with a volume mean diameter of about 95 microns to about 450 microns.

According to a particular embodiment, the immediate-release particles comprise 80.75% w/w of gamma-hydroxybutyrate, 4.25% w/w of Povidone K30 and 15% of microcrystalline cellulose spheres with a volume mean diameter of about 95 microns to about 170 microns.

According to a particular embodiment, the immediate-release particles comprise 80.75% w/w of sodium oxybate, 4.25% w/w of Povidone K30 and 15% of microcrystalline cellulose spheres.

According to another a particular embodiment, the immediate-release particles comprise 80.75% w/w of potassium salt of gamma-hydroxybutyric acid, 4.25% w/w of Povidone K30 and 15% of microcrystalline cellulose spheres.

According to another a particular embodiment, the immediate-release particles comprise 80.75% w/w of calcium salt of gamma-hydroxybutyric acid, 4.25% w/w of Povidone K30 and 15% of microcrystalline cellulose spheres.

According to another a particular embodiment, the immediate-release particles comprise 80.75% w/w of magnesium salt of gamma-hydroxybutyric acid, 4.25% w/w of Povidone K30 and 15% of microcrystalline cellulose spheres.

According to another embodiment, the immediate-release particles are manufactured by dissolving the gamma-hydroxybutyrate and the Povidone K30 in a mixture of water/ethanol 40/60 w/w and spraying the resulting solution onto the surface of the microcrystalline cellulose spheres.

### Other Characteristics of Modified Release Component

The modified release component is typically comprised of modified release particles obtained by coating immediate release particles of gamma-hydroxybutyrate with a coating (or coating film) that inhibits the immediate release of the gamma-hydroxybutyrate. In a particular subembodiment, there is no barrier coating between the gamma-hydroxybutyrate and the modified release coating. In one sub-embodiment the modified release component comprises particles comprising: (a) an inert core; (b) a coating; and (c) a layer comprising the gamma-hydroxybutyrate interposed between the core and the coating.

In a particular embodiment, the modified release component comprises a time-dependent release mechanism and a pH-dependent release mechanism, typically comprising a hydrophobic compound selected from hydrogenated vegetable oil and glyceryl tristearate and mixtures thereof and the mixture of methacrylic acid copolymers. The mixture of methacrylic acid copolymers are preferably substantially ionized at pH 7.5. The hydrophobic compound typically has a melting point equal or greater than 40° C. The hydrophobic compound and the mixture of methacrylic polymers typically constitute greater than 80%, 90%, 95%, or the entire weight of the coating.

A particularly suitable coating is composed of a mixture of hydrogenated vegetable oil and the mixture of methacrylic acid copolymers. The exact structure and amount of each component, and the amount of coating applied to the particle, controls the release rate and release triggers. Eudragit™ methacrylic acid copolymers, namely the methacrylic acid—methyl methacrylate copolymers and the methacrylic acid—ethyl acrylate copolymers, have a pH-dependent solubility: typically, the pH triggering the release of the active ingredient from the microparticles is set by the choice and mixture of appropriate Eudragit™ polymers. In the case of gamma-hydroxybutyrate modified release microparticles, the theoretical pH triggering the release is typically from 5.6 to 6.97 or 6.9, more preferably 6.5 up to 6.9. By “pH trigger” is meant the minimum pH above which dissolution of the polymer occurs.

In a particular subembodiment, the weight ratio of the hydrophobic compound to the mixture of methacrylic acid copolymers is from 0.67 to 2.33; most preferably about 1.5.

A particularly suitable coating is composed of a mixture of hydrogenated vegetable oil and methacrylic acid copolymers with a theoretical pH triggering the release from 6.5 up to 6.97 in a weight ratio from 0.67 to 2.33, most preferably of about 1.5.

The modified release particles of gamma-hydroxybutyrate typically have a volume mean diameter of from 100 to 1200 microns, from 100 to 500 microns, from 200 to 800 microns, and preferably of about 320 microns.

The coating can typically represent 10 to 50%, 15 to 45%, 20 to 40%, or 25 to 35% by weight of the total weight of the coated modified release particles. Preferably, the coating represents 25-30% by weight of the total weight of the modified release particles of gamma-hydroxybutyrate.

In a particular embodiment, the coating layer of the modified release particles of gamma-hydroxybutyrate is obtained by spraying, in particular in a fluidized bed apparatus, a solution, suspension or dispersion comprising the coating composition as defined previously onto the immediate release particles of gamma-hydroxybutyrate, in particular the immediate release particles of gamma-hydroxybutyrate as previously described. Typically, the coating is formed by spraying in a fluidized bed equipped with a Wurster or partition tube and according to an upward spray orientation or bottom spray a solution of the coating excipients in hot isopropyl alcohol.

According to a particular embodiment, the modified release particles of gamma-hydroxybutyrate consist of 10.5% w/w of microcrystalline cellulose spheres with a volume mean diameter of about 95 microns to about 450 microns, layered with 56.5% w/w of gamma-hydroxybutyrate mixed with 3% w/w of Povidone™ K30 and finally coated with a coating composition consisting of 18% w/w of hydrogenated vegetable oil (Lubritab™ or equivalent), 4% of Eudragit™ L100-55 (methacrylic acid and ethyl acrylate copolymer NF) and 8% Eudragit™ S100 (methacrylic acid and methyl methacrylate copolymer (1:2) NF), all percentages expressed based on the total weight of the final modified release particles of gamma-hydroxybutyrate.

According to a particular embodiment, the modified release particles of gamma-hydroxybutyrate consist of 10.5% w/w of microcrystalline cellulose spheres with a volume mean diameter of about 95 microns to about 170 microns, layered with 56.5% w/w of gamma-hydroxybutyrate mixed with 3% w/w of Povidone™ K30 and finally coated with a coating composition consisting of 18% w/w of hydrogenated vegetable oil (Lubritab™ or equivalent), 4% of Eudragit™ L100-55 (methacrylic acid and ethyl acrylate copolymer NF) and 8% Eudragit™ S100 (methacrylic acid and methyl methacrylate copolymer (1:2) NF), all percentages expressed based on the total weight of the final modified release particles of gamma-hydroxybutyrate.

According to a particular embodiment, the modified release particles of gamma-hydroxybutyrate consist of 10.5% w/w of microcrystalline cellulose spheres with a volume mean diameter of about 95 microns to about 450 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of Eudragit™ L100-55 (methacrylic acid and ethyl acrylate copolymer NF) and 8% Eudragit™ S100 (methacrylic acid and methyl methacrylate copolymer (1:2) NF), all percentages expressed based on the total weight of the final modified release particles of sodium oxybate.

According to a particular embodiment, the modified release particles of gamma-hydroxybutyrate consist of 10.5% w/w of microcrystalline cellulose spheres with a volume mean diameter of about 95 microns to about 170 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of Eudragit™ L100-55 (methacrylic acid and ethyl acrylate copolymer NF) and 8% Eudragit™ S100 (methacrylic acid and methyl methacrylate copolymer (1:2) NF), all percentages expressed based on the total weight of the final modified release particles of sodium oxybate.

According to another a particular embodiment, the modified release particles of gamma-hydroxybutyrate consist of 11.3% w/w of microcrystalline cellulose spheres with a volume mean diameter of about 95 microns to about 450 microns, layered with 60.5% w/w of gamma-hydroxybutyrate mixed with 3.2% w/w of Povidone™ K30 and finally coated with a coating composition consisting of 15% w/w of hydrogenated vegetable oil (Lubritab™ or equivalent), 0.75% of Eudragit™ L100-55 (methacrylic acid and ethyl acrylate copolymer NF) and 9.25% Eudragit™ S100 (methacrylic acid and methyl methacrylate copolymer (1:2) NF).

According to another a particular embodiment, the modified release particles of gamma-hydroxybutyrate consist of 11.3% w/w of microcrystalline cellulose spheres with a volume mean diameter of about 95 microns to about 170 microns, layered with 60.5% w/w of gamma-hydroxybutyrate mixed with 3.2% w/w of Povidone™ K30 and finally coated with a coating composition consisting of 15% w/w of hydrogenated vegetable oil (Lubritab™ or equivalent), 0.75% of Eudragit™ L100-55 (methacrylic acid and ethyl acrylate copolymer NF) and 9.25% Eudragit™ S100 (methacrylic acid and methyl methacrylate copolymer (1:2) NF).

According to another a particular embodiment, the modified release particles of gamma-hydroxybutyrate consist of 11.3% w/w of microcrystalline cellulose spheres with a volume mean diameter of about 95 microns to about 450 microns, layered with 60.5% w/w of sodium oxybate mixed with 3.2% w/w of Povidone™ K30 and finally coated with a coating composition consisting of 15% w/w of hydrogenated vegetable oil (Lubritab™ or equivalent), 0.75% of Eudragit™ L100-55 (methacrylic acid and ethyl acrylate copolymer NF) and 9.25% Eudragit™ S100 (methacrylic acid and methyl methacrylate copolymer (1:2) NF).

According to another a particular embodiment, the modified release particles of gamma-hydroxybutyrate consist of 11.3% w/w of microcrystalline cellulose spheres with a volume mean diameter of about 95 microns to about 170 microns, layered with 60.5% w/w of sodium oxybate mixed with 3.2% w/w of Povidone™ K30 and finally coated with a coating composition consisting of 15% w/w of hydrogenated vegetable oil (Lubritab™ or equivalent), 0.75% of Eudragit™ L100-55 (methacrylic acid and ethyl acrylate copolymer NF) and 9.25% Eudragit™ S100 (methacrylic acid and methyl methacrylate copolymer (1:2) NF).

### Dissolution Subembodiments

Additional subembodiments are defined based on the dissolution properties of the formulation. Thus, in one subembodiment (a) the composition releases at least 80% of its gamma-hydroxybutyrate at 3 hours when tested in a dissolution apparatus 2 according to USP 38<711> in 900 mL of 0.05M monobasic potassium phosphate buffer pH 6.8 at a temperature of 37° C. and a paddle speed of 75 rpm, (b) the composition releases from 10% to 65%, of its gamma-hydroxybutyrate at one hour and three hours when tested in a dissolution apparatus 2 according to USP 38 <711> in 900 mL of 0.1N hydrochloric acid at a temperature of 37° C. and a paddle speed of 75 rpm, and (c) the modified release component releases greater than 80% of its gamma-hydroxybutyrate at 3 hours in a dissolution test started in 750 ml of 0.1N hydrochloric acid for 2 hours then switched to 950 mL 0.05M monobasic potassium phosphate buffer adjusted to pH 6.8 at a temperature of 37° C. and a paddle speed of 75 rpm.

In another subembodiment (a) the immediate release component releases greater than 80% of its gamma-hydroxybutyrate at one hour when tested in a dissolution apparatus 2 according to USP 38<711> in 900 mL of 0.1N hydrochloric acid at a temperature of 37° C. and a paddle speed of 75 rpm; (b) the modified release component releases less than 20% of its gamma-hydroxybutyrate at one hour when tested in a dissolution apparatus 2 according to USP 38<711> in 900 mL of 0.1N hydrochloric acid at a temperature of 37° C. and a paddle speed of 75 rpm; (c) the modified release component releases greater than 80% of its gamma-hydroxybutyrate at three hours when tested in a dissolution apparatus 2 according to USP 38<711> in 900 mL of 0.05M monobasic potassium phosphate buffer pH 6.8 at a temperature of 37° C. and a paddle speed of 75 rpm; and (d) the modified release component releases greater than 80% of its gamma-hydroxybutyrate at 3 hours in a dissolution test started in 750 mL of 0.1N hydrochloric acid for 2 hours then switched to 950 mL 0.05M monobasic potassium phosphate buffer adjusted to pH 6.8 at a temperature of 37° C. and a paddle speed of 75 rpm.

In another subembodiment the modified release component releases greater than 80% of its gamma-hydroxybutyrate at one hour when tested in a dissolution apparatus 2 according to USP 38<711> in 900 mL of 0.05M monobasic potassium phosphate buffer pH 6.8 at a temperature of 37° C. and a paddle speed of 75 rpm.

In a preferred embodiment, the formulation of gamma-hydroxybutyrate according to the invention achieves an in vitro dissolution profile:
(a) measured in a dissolution apparatus 2 according to USP 38<711> in 900 mL of 0.1N hydrochloric acid at a temperature of 37° C. and a paddle speed of 75 rpm, characterized by the percentage of gamma-hydroxybutyrate dissolved being:
   (i) from 40% to 65% at 1 hour,
   (ii) from 40% to 65% at 3 hours,
   (iii) from 47% to 85% at 8 hours,
   (iv) greater or equal to 60% at 10 hours,
   (v) greater or equal to 80% at 16 hours, and
(b) measured in a dissolution apparatus 2 according to USP 38<711> in 900 mL of 0.05M monobasic potassium phosphate buffer pH 6.8 at a temperature of 37° C. and a paddle speed of 75 rpm, characterized by the percentage of gamma-hydroxybutyrate dissolved being:
   (i) from 43% to 94% at 0.25 hour,
   (ii) greater or equal to 65% at 0.5 hour, and
   (iii) greater or equal to 88% at 1 hour.

In a preferred embodiment, the formulation of gamma-hydroxybutyrate according to the invention achieves an in vitro dissolution profile:
(a) measured in a dissolution apparatus 2 according to USP 38<711> in 900 mL of 0.1N hydrochloric acid at a temperature of 37° C. and a paddle speed of 75 rpm, characterized by the percentage of gamma-hydroxybutyrate dissolved being:
   (i) from 40% to 65% at 1 hour,
   (ii) from 40% to 65% at 3 hours,
   (iii) greater or equal to 47% at 8 hours,
   (iv) greater or equal to 60% at 10 hours,
   (v) greater or equal to 80% at 16 hours, and
(b) measured in a dissolution apparatus 2 according to USP 38<711> in 900 mL of 0.05M monobasic potassium phosphate buffer pH 6.8 at a temperature of 37° C. and a paddle speed of 75 rpm, characterized by the percentage of gamma-hydroxybutyrate dissolved being:
   (i) from 43% to 94% at 0.25 hour,
   (ii) greater or equal to 65% at 0.5 hour, and (iii) greater or equal to 88% at 1 hour.

In another preferred embodiment, the formulation of gamma-hydroxybutyrate according to the invention achieves an in vitro dissolution profile:
(a) measured in a dissolution apparatus 2 according to USP 38<711> in 900 mL of 0.1N hydrochloric acid at a temperature of 37° C. and a paddle speed of 75 rpm, characterized by the percentage of gamma-hydroxybutyrate dissolved being:
   (i) from 40% to 65% at 1 hour,
   (ii) from 40% to 65% at 3 hours,
   (iii) from 47% to 85% at 8 hours,
   (iv) greater or equal to 60% at 10 hours,
   (v) greater or equal to 80% at 16 hours, and
(b) measured in a dissolution apparatus 2 according to USP 38<711> in 900 mL of 0.05M monobasic potassium phosphate buffer pH 6.8 at a temperature of 37° C. and a paddle speed of 75 rpm, characterized by the percentage of gamma-hydroxybutyrate dissolved being:
   (i) from 45% to 67% at 1 hour, and
   (ii) greater or equal to 65% at 3 hours.

In another preferred embodiment, the formulation of gamma-hydroxybutyrate according to the invention achieves an in vitro dissolution profile:
(a) measured in a dissolution apparatus 2 according to USP 38<711> in 900 mL of 0.1N hydrochloric acid at a temperature of 37° C. and a paddle speed of 75 rpm, characterized by the percentage of gamma-hydroxybutyrate dissolved being:
   (i) from 40% to 65% at 1 hour,
   (ii) from 40% to 65% at 3 hours,
   (iii) greater or equal to 47% at 8 hours,
   (iv) greater or equal to 60% at 10 hours,
   (v) greater or equal to 80% at 16 hours, and
(b) measured in a dissolution apparatus 2 according to USP 38<711> in 900 mL of 0.05M monobasic potassium phosphate buffer pH 6.8 at a temperature of 37° C. and a paddle speed of 75 rpm, characterized by the percentage of gamma-hydroxybutyrate dissolved being:
   (i) from 45% to 67% at 1 hour, and
   (ii) greater or equal to 65% at 3 hours.

In yet another subembodiment (a) the modified release component releases greater than 80% of its gamma-hydroxybutyrate at 3 hours in a dissolution test started in 750 mL of 0.1N hydrochloric acid for 2 hours then switched to 950 mL 0.05M monobasic potassium phosphate buffer adjusted to pH 6.8 at a temperature of 37° C. and a paddle speed of 75 rpm; and (b) the immediate release component releases greater than 80% of its gamma-hydroxybutyrate at one hour when tested in a dissolution apparatus 2 according to USP 38<711> in 900 mL of 0.1N hydrochloric acid at a temperature of 37° C. and a paddle speed of 75 rpm.

In another subembodiment (a) a 7.5 g dose of the composition has been shown to achieve a mean AUC_{inf} of greater than 340 hr·microgram/mL, and a mean Con that is less than 200% (optionally from 50% to 130%) of the mean C₈ₕ provided by an equal dose of an immediate release liquid solution of sodium oxybate administered at to and t₄ₕ in equally divided doses approximately two hours after a standardized evening meal, and (b) the composition releases (i) at least 80% of its gamma-hydroxybutyrate at 3 hours when tested in a dissolution apparatus 2 according to USP 38<711> in 900 mL of 0.05M monobasic potassium phosphate buffer pH 6.8 at a temperature of 37° C. and a paddle speed of 75 rpm, and (ii) from 10% to 65%, of its gamma-hydroxybutyrate at one hour and three hours when tested in a dissolution apparatus 2 according to USP 38<711> in 900 mL of 0.1N hydrochloric acid at a temperature of 37° C. and a paddle speed of 75 rpm, and (c) the modified release component releases greater than 80% of its gamma-hydroxybutyrate at 3 hours in a dissolution test started in 750 mL of 0.1N hydrochloric acid for 2 hours then switched to 950 mL 0.05M monobasic potassium phosphate buffer adjusted to pH 6.8 at a temperature of 37° C. and a paddle speed of 75 rpm.

In yet another subembodiment the composition comprises immediate release and modified release components, wherein (a) said immediate release component releases greater than 80% of its gamma-hydroxybutyrate at one hour when tested in a dissolution apparatus 2 according to USP 38<711> in 900 mL of 0.1N hydrochloric acid at a temperature of 37° C. and a paddle speed of 75 rpm; (b) said modified release component releases less than 20% of its gamma-hydroxybutyrate at one hour when tested in a dissolution apparatus 2 according to USP 38<711> in 900 mL of 0.1N hydrochloric acid at a temperature of 37° C. and a paddle speed of 75 rpm; (c) said modified release component releases greater than 80% of its gamma-hydroxybutyrate at three hours when tested in a dissolution apparatus 2 according to USP 38<711> in 900 mL of 0.05M monobasic potassium phosphate buffer pH 6.8 at a temperature of 37° C. and a paddle speed of 75 rpm; and (d) said modified release component releases greater than 80% of its gamma-hydroxybutyrate at 3 hours in a dissolution test started in 750 mL of 0.1N hydrochloric acid for 2 hours then switched to 950 mL 0.05M monobasic potassium phosphate buffer adjusted to pH 6.8 at a temperature of 37° C. and a paddle speed of 75 rpm.

In another subembodiment the composition releases (a) at least 80% of its gamma-hydroxybutyrate at three hours when tested in a dissolution apparatus 2 according to USP 38<711> in 900 mL of 0.05M monobasic potassium phosphate buffer pH 6.8 at a temperature of 37° C. and a paddle speed of 75 rpm, and (b) from 10% to 65%, of its gamma-hydroxybutyrate at one hour and three hours when tested in a dissolution apparatus 2 according to USP 38<711> in 900 mL of 0.1N hydrochloric acid at a temperature of 37° C. and a paddle speed of 75 rpm.

In another subembodiment the composition comprises immediate release and modified release components, wherein: (a) the composition releases at least 80% of its gamma-hydroxybutyrate at 3 hours when tested in a dissolution apparatus 2 according to USP 38<711> in 900 mL of 0.05M monobasic potassium phosphate buffer pH 6.8 at a temperature of 37° C. and a paddle speed of 75 rpm, (b) the composition releases 10% to 65% of its gamma-hydroxybutyrate at one hour and at three hours when tested in a dissolution apparatus 2 according to USP 38<711> in 900 mL of 0.1N hydrochloric acid at a temperature of 37° C. and a paddle speed of 75 rpm, (c) the composition releases greater than 60% of its gamma-hydroxybutyrate at 10 hours when tested in a dissolution apparatus 2 according to USP 38<711> in 900 mL of 0.1N hydrochloric acid at a temperature of 37° C. and a paddle speed of 75 rpm, and (d) the modified release component releases greater than 80% of its gamma-hydroxybutyrate at 3 hours in a dissolution test started in 750 ml of 0.1N hydrochloric acid for 2 hours then switched to 950 mL 0.05M monobasic potassium phosphate buffer adjusted to pH 6.8 at a temperature of 37° C. and a paddle speed of 75 rpm.

### Pharmacokinetic Embodiments and Subembodiments

The compositions of the present invention can also be defined in terms of pharmacokinetics, optionally in combination with any of the foregoing dissolution or structural characteristics. Thus, in one pharmacokinetic embodiment or subembodiment the invention provides a composition of gamma-hydroxybutyrate, wherein a 7.5 g dose of the formulation has been shown to achieve a mean AUC_{inf} of greater than 340 hr·microgram/mL, and a mean Con that is less than 200% of the mean C₈ₕ provided by an equal dose of immediate release liquid solution of sodium oxybate administered at t₀ and t₄ₕ in equally divided doses approximately two hours after a standardized evening meal.

In another pharmacokinetic embodiment or subembodiment the invention provides a composition of gamma-hydroxybutyrate, comprising immediate release and modified release portions, wherein (a) a 7.5 g dose of the formulation has been shown to achieve a mean AUC_{inf} of greater than 340 hr·microgram/mL, and a mean C₈ₕ that is less than 200%, of the mean C₈ₕ provided by an equal dose of an immediate release liquid solution of sodium oxybate administered at to and t₄ₕ in equally divided doses approximately two hours after a standardized evening meal, and (b) the formulation releases (i) at least 80% of its gamma-hydroxybutyrate at 3 hours when tested in a dissolution apparatus 2 according to USP 38<711> in 900 mL of 0.05M monobasic potassium phosphate buffer pH 6.8 at a temperature of 37° C. and a paddle speed of 75 rpm, and (ii) from 10% to 65%, of its gamma-hydroxybutyrate at one hour and three hours when tested in a dissolution apparatus 2 according to USP 38<711> in 900 mL of 0.1N hydrochloric acid at a temperature of 37° C. and a paddle speed of 75 rpm, and (c) the modified release portion releases greater than 80% of its gamma-hydroxybutyrate at 3 hours in a dissolution test started in 750 ml of 0.1N hydrochloric acid for 2 hours then switched to 950 mL 0.05M monobasic potassium phosphate buffer adjusted to pH 6.8 at a temperature of 37° C. and a paddle speed of 75 rpm.

In any of the embodiments of the invention a 7.5 g dose of the formulation has been shown to achieve a mean C₈ₕ that is less than 100%, 75%, 50%, or 45% of the mean C₈ₕ provided by an equal dose of an immediate release liquid solution of sodium oxybate administered at t₀ and t₄ₕ in equally divided doses approximately two hours after a standardized evening meal. Alternatively or in addition, a 4.5 g, 6 g, 7.5 g, or 9 g dose of the formulation has been shown to achieve a relative bioavailability (RBA) of greater than 80% when compared to an equal dose of an immediate release liquid solution of sodium oxybate administered at t₀ and t₄ₕ in equally divided doses, when administered approximately two hours after a standardized evening meal.

In another pharmacokinetic embodiment or subembodiment a 7.5 g dose of the composition has been shown to achieve a mean AUC_{inf} of greater than 340 hr·microgram/mL. In still another pharmacokinetic embodiment or subembodiment a 7.5 g dose of the composition has been shown to achieve a mean AUC_{inf} of greater than 340 hr·microgram/mL, and a mean C₈ₕ that is less than 130% of the mean C₈ₕ provided by an equal dose of immediate release liquid solution of sodium oxybate administered at t₀ and t₄ₕ in equally divided doses approximately two hours after a standardized evening meal. In yet another pharmacokinetic embodiment or subembodiment a 4.5 g and a 9 g dose of the composition has been shown to achieve a relative bioavailability (RBA) of greater than 80% when compared to an equal dose of an immediate release liquid solution of sodium oxybate administered at t₀ and t₄ₕ in equally divided doses, when administered approximately two hours after a standardized evening meal.

### Methods of Treatment

The invention further provides a method of treating a disorder treatable with gamma-hydroxybutyrate in a human subject in need thereof comprising orally administering a single bedtime daily dose to said human amounts of gamma-hydroxybutyrate equivalent to from 3.0 to 12.0 g of sodium oxybate in the formulation of the present invention. The invention further provides methods of treating narcolepsy, types 1 and/or 2, by orally administering at bedtime a therapeutically effective amount of a gamma-hydroxybutyrate formulation characterized by the novel gamma-hydroxybutyrate dissolution properties of the present invention. The formulation of the present invention is effective to treat narcolepsy Type 1 or Type 2, wherein said treatment of narcolepsy is defined as reducing excessive daytime sleepiness or reducing the frequency of cataplectic attacks. The therapeutically effective amount typically comprises equivalents from 3.0 to 12.0 g of sodium oxybate, more preferably from to 9.0 g of sodium oxybate, and most preferably 4.5, 6.0, 7.5 or 9.0 g of sodium oxybate.

In general, the method comprises opening the packaged solid composition, contacting the solid composition with a suitable liquid, mixing the solid composition and liquid to form a mixture (e.g., a suspension), and orally administering the mixture to an individual in need thereof. The solid composition may be added to a glass or other container containing the liquid, the solid composition may be added to a glass or other container and then liquid may be added to the glass or container, or the liquid may be added to the package comprising the solid composition. The solid composition and the liquid are then mixed to form a mixture, wherein the mixing comprises stirring, shaking, agitating, blending, inverting, or other suitable means for mixing the components. The liquid typically is water (i.e., tap water or drinking water, which can be still or bubbly, flavored or unflavored), but other liquids (e.g., fruit juice, carbonated soda, etc.) can be used. The amount of liquid mixed with the solid composition may vary. For example, the amount of liquid may range from about 30 mL to about 100 mL, or, for example, about 50 mL.

### EXAMPLES

### Example 1: Method of Manufacturing Formulations Used in the Succeeding Examples

The two formulations used in the succeeding examples and their manufacturing processes are given below. Test results from these two different formulations were practically indistinguishable.

### First Formulation

Tables 1a-1d provide the qualitative and quantitative compositions of sodium oxybate IR microparticles, MR microparticles, and mixtures of IR and MR microparticles, of the first formulation. The physical structure of the microparticles showing the qualitative and quantitative composition of the IR and MR microparticles is depicted in FIG. 1.

Briefly, sodium oxybate immediate release (IR) microparticles were prepared as follows: 1615.0 g of sodium oxybate and 85.0 g of polyvinylpyrrolidone (Povidone K30-Plasdone™ K29/32 from ISP) were solubilized in 1894.3 g of absolute ethyl alcohol and 1262.9 g of water. The solution was entirely sprayed onto 300 g of microcrystalline cellulose spheres (Cellets™ 127) in a fluid bed spray coater apparatus. IR Microparticles with volume mean diameter of about 270 microns were obtained.

Sodium oxybate modified release (MR) microparticles were prepared as follows: 22.8 g of Eudragit™ L100-55, 45.8 g of Eudragit™ S100, 102.9 g of hydrogenated cottonseed oil (Lubritab™), were dissolved in 1542.9 g of isopropanol at 78° C. The solution was sprayed entirely onto 400.0 g of the sodium oxybate IR microparticles described above in a fluid bed spray coater apparatus with an inlet temperature of 48° C., spraying rate around 11 g per min and atomization pressure of 1.3 bar. MR microparticles were dried for two hours with inlet temperature set to 56° C. MR microparticles with mean volume diameter of about 320 microns were obtained.

The finished composition, which contains a 50:50 mixture of MR and IR microparticles calculated on their sodium oxybate content, was prepared as follows: 353.36 g of the above IR microparticles, 504.80 g of the above MR microparticles, 14.27 g of malic acid (D/L malic acid), 6.34 g of xanthan gum (Xantural™ 75 from Kelco), 9.51 g of carrageenan gum (Viscarin™ PH209 from FMC Biopolymer), 9.51 g of hydroxyethylcellulose (Natrosol™ 250M from Ashland) and 4.51 g of magnesium stearate were mixed. Individual samples of 7.11 g (corresponding to a 4.5 g dose of sodium oxybate with half of the dose as immediate-release fraction and half of the dose as modified release fraction) were weighed.

TABLE 1a
Composition of IR Microparticles

| | | Quantity per |
|---|---|---|
| Component | Function | 2.25 g dose (g) |
| Sodium oxybate | Drug substance | 2.25 |
| Microcrystalline | Core | 0.418 |
| cellulose spheres | | |
| Povidone K30 | Binder and excipient | 0.118 |
| | in diffusion coating | |
| Ethyl alcohol | Solvent | Eliminated during |
| | | processing |
| Purified water | Solvent | Eliminated during |
| | | processing |
| Total | | 2.786 |

TABLE 1b
Composition of MR Microparticles

| | | Quantity per |
|---|---|---|
| Component | Function | 4.5 g dose (g) |
| IR Microparticles | Core of MR | 2.786 |
| | microparticles | |
| Hydrogenated | Coating excipient | 0.716 |
| Vegetable Oil | | |
| Eudragit ™ L100-55 | Coating excipient | 0.159 |
| Eudragit ™ S100 | Coating excipient | 0.318 |
| Isopropyl alcohol | Solvent | Eliminated during |
| | | processing |
| Total | | 3.981 |

TABLE 1c
Qualitative Finished Composition

| | | Quantity per |
|---|---|---|
| Component | Function | 4.5 g dose (g) |
| MR microparticles | Modified release fraction | 3.981 |
| | of sodium oxybate | |
| IR microparticles | Immediate release fraction | 2.786 |
| | of sodium oxybate | |
| Malic acid | Acidifying agent | 0.113 |
| Xanthan gum | Suspending agent | 0.050 |
| Hydroxyethylcellulose | Suspending agent | 0.075 |
| Carrageenan gum | Suspending agent | 0.075 |
| Magnesium stearate | Lubricant | 0.036 |
| Total | | 7.116 |

TABLE 1d
Quantitative finished composition

| | | Quantity per |
|---|---|---|
| Component | Function | 4.5 g dose (g) |
| Sodium oxybate | Drug substance | 4.5 |
| Microcrystalline | Core | 0.836 |
| cellulose spheres | | |
| Povidone K30 | Binder | 0.237 |
| Hydrogenated | Coating excipient | 0.716 |
| Vegetable Oil | | |
| Eudragit ™ L100-55 | Coating excipient | 0.159 |
| Eudragit ™ S100 | Coating excipient | 0.318 |
| Malic acid | Acidifying agent | 0.113 |
| Xanthan gum | Suspending agent | 0.050 |
| Hydroxyethylcellulose | Suspending agent | 0.075 |
| Carrageenan gum | Suspending agent | 0.075 |
| Magnesium stearate | Lubricant | 0.036 |
| Total | | 7.116 |

### Second Formulation

The second formulation and its manufacturing process is described as follows. Briefly, sodium oxybate immediate release (IR) microparticles were prepared by coating the IR microparticles of the first process with a top coat layer. Microparticles were prepared as follows: 170.0 of hydroxypropyl cellulose (Klucel™ EF Pharm from Hercules) were solubilized in 4080.0 g of acetone. The solution was entirely sprayed onto 1530.0 g of the IR microparticles of the first process in a fluid bed spray coater apparatus. IR Microparticles with volume mean diameter of about 298 microns were obtained (see Table 1e).

Sodium oxybate modified release (MR) microparticles were prepared as described in the first process (see Table 1b).

The finished composition, which contains a 50:50 mixture of MR and IR microparticles based on their sodium oxybate content, was prepared as follows: 412.22 g of the above IR microparticles, 530.00 g of the above MR microparticles, 29.96 g of malic acid (D/L malic acid), 4.96 g of xanthan gum (Xantural™ 75 from Kelco), 4.96 g of colloidal silicon dioxide (Aerosil™ 200 from Degussa) and 9.92 g of magnesium stearate were mixed. Individual samples of 7.45 g (corresponding to a 4.5 g dose of sodium oxybate with half of the dose in an immediate-release fraction and half of the dose in a modified release fraction) were weighed (see Tables 1f and 1e).

TABLE 1e
Composition of IR Microparticles

| | | Quantity per |
|---|---|---|
| Component | Function | 2.25 g dose (g) |
| Sodium oxybate | Drug substance | 2.25 |
| Microcrystalline | Core | 0.418 |
| cellulose spheres | | |
| Povidone K30 | Binder and excipient | 0.118 |
| | in diffusion coating | |
| Hydroxypropyl cellulose | Top coat | 0.310 |
| Ethyl alcohol | Solvent | Eliminated during |
| | | processing |
| Purified water | Solvent | Eliminated during |
| | | processing |
| Acetone | Solvent | Eliminated during |
| | | processing |
| Total | | 3.096 |

TABLE 1f
Qualitative Finished Composition

| | | Quantity per |
|---|---|---|
| Component | Function | 4.5 g dose (g) |
| MR microparticles | Modified release fraction | 3.981 |
| | of sodium oxybate | |
| IR microparticles | Immediate release | 3.096 |
| | fraction of sodium | |
| | oxybate | |
| Malic acid | Acidifying agent | 0.225 |
| Xanthan gum | Suspending agent | 0.037 |
| Colloidal silicon dioxide | Gliding agent | 0.037 |
| Magnesium stearate | Lubricant | 0.075 |
| Total | | 7.451 |

TABLE 1g
Quantitative finished composition

| | | Quantity per |
|---|---|---|
| Component | Function | 4.5 g dose (g) |
| Sodium oxybate | Drug substance | 4.5 |
| Microcrystalline | Core | 0.836 |
| cellulose spheres | | |
| Povidone K30 | Binder | 0.237 |
| Hydroxypropyl cellulose | Top coat | 0.310 |
| Hydrogenated | Coating excipient | 0.716 |
| Vegetable Oil | | |
| Eudragit ™ L100-55 | Coating excipient | 0.159 |
| Eudragit ™ S100 | Coating excipient | 0.318 |
| Malic acid | Acidifying agent | 0.225 |
| Xanthan gum | Suspending agent | 0.037 |
| Colloidal silicon dioxide | Gliding agent | 0.037 |
| Magnesium stearate | Lubricant | 0.075 |
| Total | | 7.451 |

Compared to the first formulation, the second formulation has the following characteristics: same MR microparticles, same IR microparticles but with a top coat, increased amount of malic acid, only one suspending agent (xanthan gum) and presence of a glidant.

### Example 2: Method of Evaluating Dissolution Stability of Exemplary Formulations

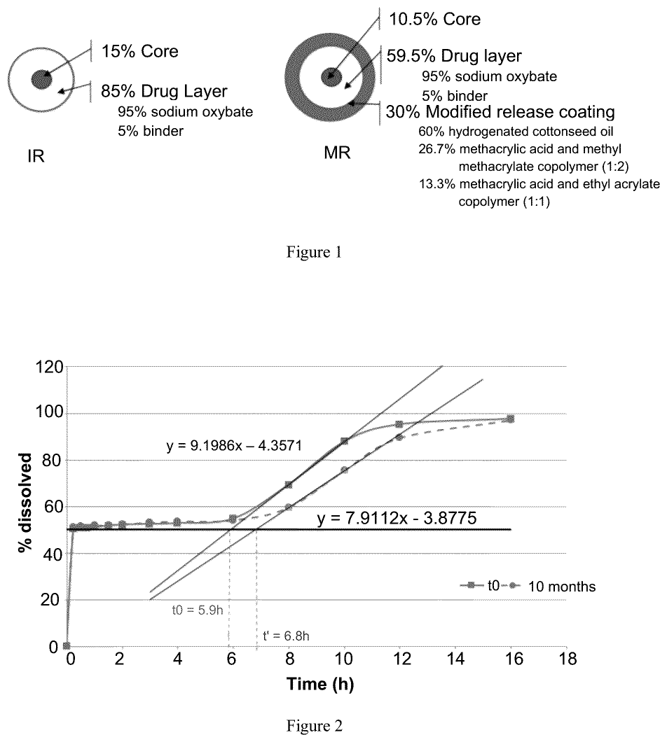

An analysis was undertaken to evaluate the dissolution stability of packaged formulations containing 50% of the sodium oxybate dose in immediate release particles and 50% of the sodium oxybate dose in modified release particles, corresponding to the second formulation in example 1. The formulation was packaged in DUMA™ bottles with 2 g silica gel desiccant. The formulation was tested in a dissolution apparatus 2 according to USP 38<711> in 0.1N hydrochloric acid at a temperature of 37° C. and a paddle speed of 75 rpm. Single dose units were poured into a container containing 50 mL of tap water and shaken to form a suspension. After 5 minutes, the suspension was poured into a dissolution vessel containing 840 mL of 0.1N HCl dissolution medium. 10 ml of water were then used to rinse the container and subsequently added to the dissolution vessel. A sample of the formulation was tested shortly after it was prepared at month 0, and subsequently tested one month later after storage at 40° C. and 75% relative humidity. The percent dissolved at various time points is reported in Tables 2A (month 0) and 2B (one month) and depicted in FIG. 2.

TABLE 2A

| | % dissolved |
|---|---|
| | in 0.1N |
| Time (h) | HCl at t0 |
| 0.0 | 0 |
| 0.3 | 50 |
| 0.5 | 51 |
| 1.0 | 51 |
| 1.5 | 52 |
| 2.0 | 52 |
| 3.0 | 52 |
| 4.0 | 53 |
| 6.0 | 55 |
| 8.0 | 69 |
| 10.0 | 88 |
| 12.0 | 95 |
| 16.0 | 97 |

TABLE 2B

| | % |
|---|---|
| | dissolved in |
| | 0.1N HCl |
| | after one |
| | month at |
| | 40° C./ |
| Time (h) | 75% RH |
| 0.0 | 0 |
| 0.3 | 51 |
| 0.5 | 51 |
| 1.0 | 52 |
| 1.5 | 52 |
| 2.0 | 52 |
| 3.0 | 53 |
| 4.0 | 54 |
| 6.0 | 54 |
| 8.0 | 59 |
| 10.0 | 75 |
| 12.0 | 89 |
| 16.0 | 97 |

For this and succeeding Examples, the formulation was considered stable on dissolution testing if the absolute value (t′−t₀) was less than 0.83 h (=50 min) and/or the difference of API dissolved at all dissolution sampling times was less than 10%. t₀ was determined by drawing a horizontal line across the y-axis at 50% dissolved, corresponding to the percentage of sodium oxybate dose present in the immediate release fraction. A first tangent was then drawn on the month 0 release profile between two time points two hours apart corresponding to the rate of greatest release. The intersection between the first tangent and the horizontal was assigned to. A second tangent was then drawn on the month 1 release profile between the two (2) time points separated by two hours corresponding to the rate of greatest release on the month 1 release profile. The intersection between the second tangent and the horizontal line was assigned t′.

In this example, after just one month, t′−t₀ equaled 0.9 h, which was greater than the 0.83 h (=50 min) pre-specified criteria. In addition, the difference in API dissolved at t₈ₕ and t₁₀ₕ was greater than 10%. As a consequence, the formulation in this packaging was considered unstable on dissolution testing.

While this formulation showed instability after just one month, more typically, evolution of the dissolution behavior after two months will be more probative of the long-term stability of the formulation. As a consequence, a formulation can also be defined as stable if, after at least 2 months at 40° C./75% RH: the absolute value (t′−t₀) is less than 0.83 h (=50 min) and/or the difference in active ingredient dissolved at any dissolution sampling times is less than 10%.

Most preferably, however, the formulation will be evaluated over its entire shelf life in real world storage conditions. Thus, a formulation is most preferably defined as stable if, after at least 18 or 24 months at 25° C./60% RH or 30° C./65% RH: the absolute value (t′−t₀) is less than 0.83 h (=50 min) and/or the difference in active ingredient dissolved at any dissolution sampling times is less than 10%.

### Example 3: Evaluation of Effect of Packaging Type on Dissolution Stability

In order to determine the effect of packaging type on the dissolution stability of the formulations of the present invention, a formulation manufactured according to Example 1 (first formulation) was packaged in various containers and evaluated via dissolution testing according to the method described in Example 2. The results of the testing are reported in Table 3:

TABLE 3

| | | | Max of |
|---|---|---|---|
| | | | Δ (% API |
| Supplier | | t’-t₀ | dissolved) as |
| (type of | Packaging | as described | described in |
| packaging) | references | in example 2 | example 2* |
| Bischof & Klein | PET/ALU/PE | 0.24 h (3 months | 2 (3 months |
| (sachets) | with 9 μm ALU foil | dissolution data | dissolution data |
| | | considered) | considered) |
| Constantia (stick-pack) | PET/adhesive | 0.1 h (3 months | 4 (3 months |
| | layer/ALU/ | dissolution data | dissolution data |
| | copolymer with | considered) | considered) |
| | 12 μm ALU foil | | |
| LOG ™ | Bottle: H2OO2: | −0.54 h (3 months | 4 (3 months |
| | 40 ml White Bot. | dissolution data | dissolution data |
| | 33/400 MBF 20 | considered) | considered) |
| | Cap: 33 mm White | | |
| | CR Cap + IHS | | |
| | liner | | |
| Gerresheimer/ | Bottle: | −2.02 h (2 months | 17 (2 months |
| DUMA ™ w/o | 035030-3000 | dissolution data | dissolution data |
| desiccant (bottles) | 30 ml bottle | considered) | considered) |
| | Cap: | | |
| | 02827D-3000 | | |
| Gerresheimer/ | Bottle: | 0.9 h (1 month | 12 (1 month |
| DUMA ™ with 2 g | 035030-3000 | dissolution data | dissolution data |
| desiccant (bottles) | 30 ml bottle | considered) | considered) |
| | Cap (without | | |
| | desiccant): | | |
| | 02827D-3000 | | |
| | Desiccant: 2*1 g | | |
| | silica gel Minipax | | |
| | OR | | |
| | Cap (with inserted | | |
| | desiccant): | | |
| | 02827T-300T | | |
| Gerresheimer/ | Bottle: | 0.88 (1 month | 11 (1 month |
| DUMA ™ with | 035030-3000 | dissolution data | dissolution data) |
| 2 × 1 g Minipax | 30 ml bottle | considered) | |
| Intelisorb ™ | Cap: | | |
| | 02827D-3000 | | |
| | Desiccant: | | |
| | Intelisorb - 1.0 g | | |
| | IntelliSorb ® | | |
| | MR-20 | | |

*corresponds to the maximum difference of API dissolved (in %) at a given dissolution sampling time at month 0 and during stability.

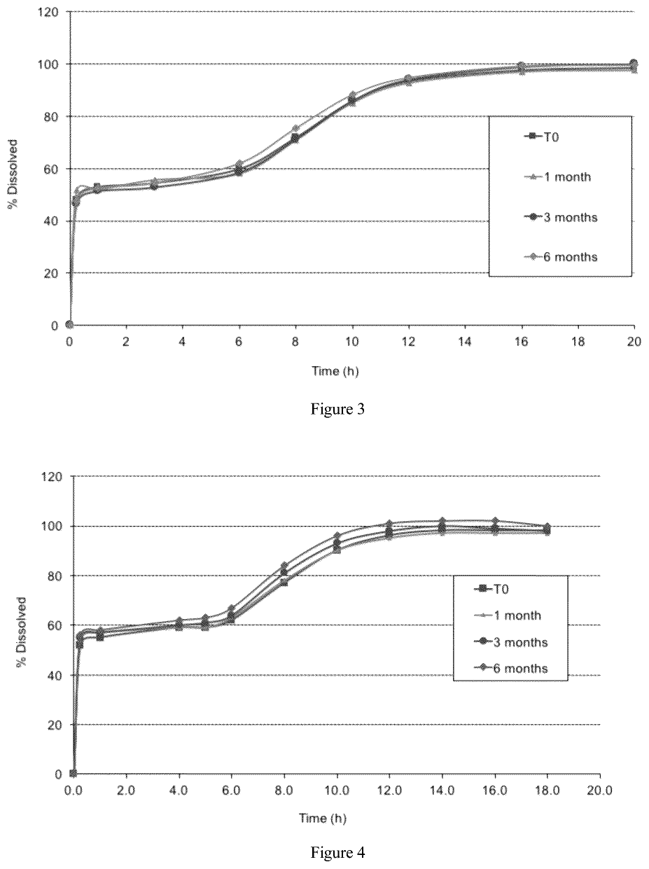

The dissolution profiles of packaged compositions from Bischof & Klein (Lengerich Germany) sachets, Constantia stick-packs and LOG bottles are respectively illustrated on FIGS. 3, 4 and 5. According to the dissolution criteria expressed in example 2 and the data listed in table 3, the three packaged compositions are stable. The dissolution profiles of packaged compositions comprising Gerresheimer Duma bottles with and without desiccant are illustrated based on additional experiments in Example 4.

### Example 4: Determination of Stabilizing Humidity Range

Based on early studies indicating that absorption of water by the formulation impacted the stability of the formulation's dissolution profile, a study was undertaken to determine whether the humidity at which the formulation was packaged would influence the dissolution stability. Drug units were packaged in sachets by sealing drug units in different conditions of relative humidity from dry to humid conditions. Regardless of the relative humidity at the time of sealing, no modification of the dissolution profile was observed after 2 months at 40° C./75% RH.

Based on these results, it was determined that the humidity inside the packaging, including any ingress or egress of humidity during storage, dictates the stability of the dissolution profile, and testing was undertaken to quantify the effect of humidity inside the packaging on the stability of the dissolution profile. Small temperature and humidity probes (Tomprobe™ from BioMerieux™) were used to assess relative humidity inside the different types of packaging.

Five stability studies were launched at 40° C./75% RH to provide RH data in the different types of packaging already investigated:
sealed aluminum sachet (PET/AI/PE) from BK (Bischof+Klein)
LOG™ H2OO2 bottle (with a barrier layer for low water vapor permeability) closed with a child proof cap
closed DUMA™ bottle with child-proof cap
closed DUMA™ bottle with 2 g desiccant (silica gel)
and closed DUMA™ bottle with 2 g INTELISORB™ Desiccant

In parallel, dissolution profiles were determined during the stability study to provide additional and complementary dissolution data to the data contained in Example 3.

### Dissolution Profile Test Results

Dissolution testing was assessed according to the method described in Example 2. The results of the testing are reported in Table 4.

LOG™ H2OO2 bottle

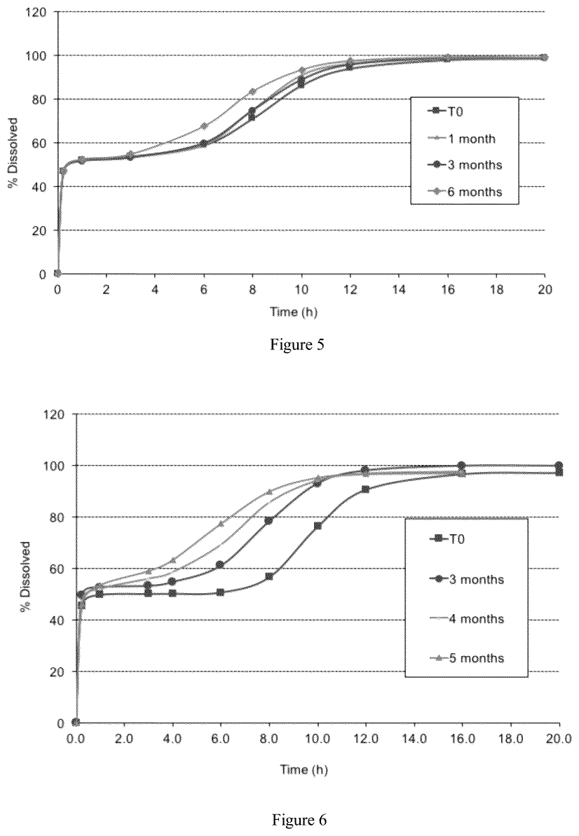

Because an unchanged dissolution profile was previously observed for this packaging after 3 months at 40° C./75% RH and an acceleration of the dissolution profile observed after 6 months (FIG. 5), it was decided to determine the dissolution profile and measure RH at 3, 4, 5 and 6 months to determine the upper RH threshold. As shown in the FIG. 6, an acceleration of the dissolution profile is unexpectedly observed after 3 months at 40° C./75% RH. The behavior after 3 months at 40° C./75% RH is therefore erratic and it was concluded that the RH at 3 months was too high to ensure formulation stability in a reproducible way.

DUMA™ Bottle without Desiccant

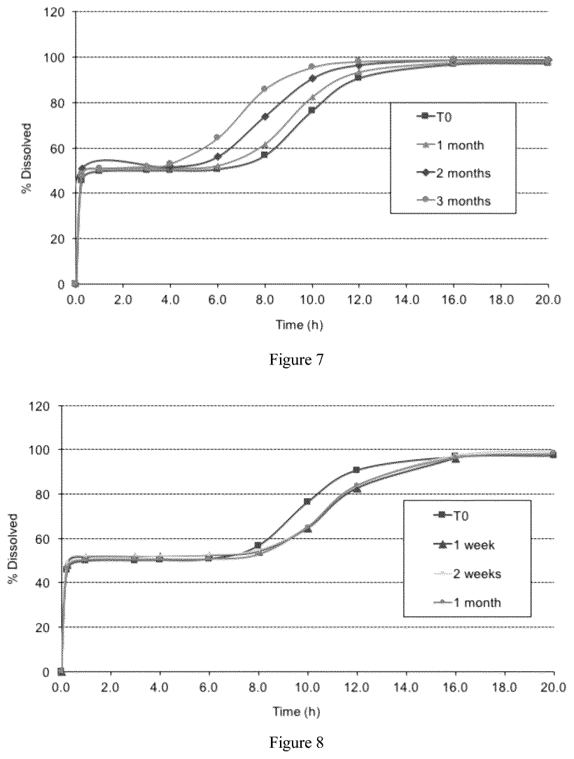

The dissolution profile in this packaging is stable after one month at 40° C./75% RH and unstable after 2 months (acceleration of the dissolution profile) as illustrated on FIG. 7. A comparison of the relative humidity in the packaging after one month and 2 months will help determine an upper limit for the RH.

DUMA™ Bottle (30 ml) with 2 g Silica Gel

As shown in FIG. 8, a slowdown of the dissolution profile was observed in this packaging after one week with no further evolution of the dissolution profile for up to one month. The dissolution profile was judged to be unstable based on the criteria in Example 2 after one week.

DUMA™ Bottle (30 ml) with 2 g Intelisorb

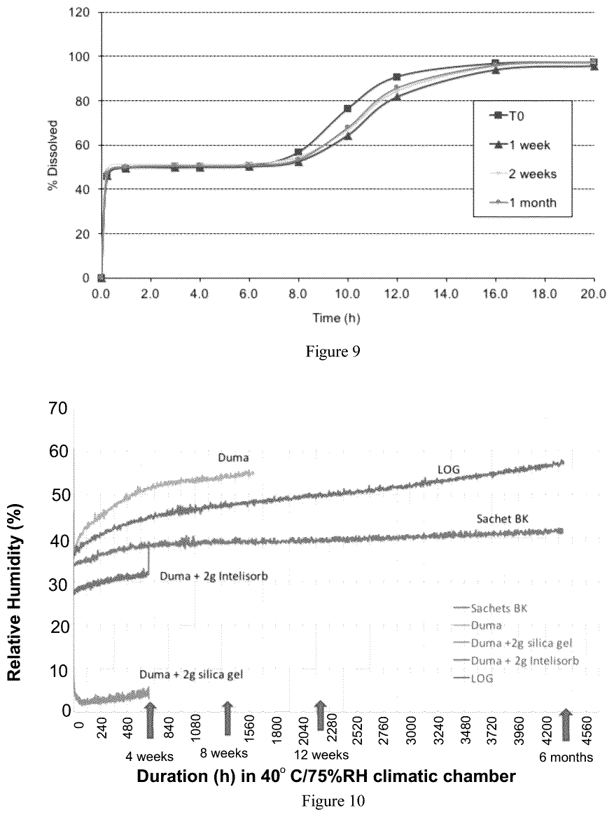

As shown in FIG. 9, a slowdown of the dissolution profile was observed in this packaging after one week with no further evolution of the dissolution profile for up to one month. The dissolution profile was judged to be unstable based on the criteria in Example 2 after one week.

TABLE 4

| | | | Max of |
|---|---|---|---|
| | | | Δ (% API |
| Supplier | | t’-t₀ | dissolved) as |
| (type of | Packaging | as described | described in |
| packaging) | references | in example 2 | example 2 |
| LOG ™ | Bottle: H2OO2: | −2.60 h (3 months | 22 (3 months |
| | 40 ml White Bot. | data considered) | considered) |
| | 33/400 MBF 20 | | |
| | Cap: 33 mm White | | |
| | CR Cap + IHS | | |
| | liner | | |
| Gerresheimer/ | Bottle: | −2.02 h (2 months | 17 (2 months |
| DUMA ™ w/o | 035030-3000 | dissolution data | dissolution data |
| desiccant (bottles) | 30 ml bottle | considered) | considered) |
| | Cap: | | |
| | 02827D-3000 | | |
| Gerresheimer/ | Bottle: | 1.06 h (1 week | 12 (1 week |
| DUMA ™ with 2 g | 035030-3000 | dissolution data | dissolution data |
| desiccant (bottles) | 30 ml bottle | considered) | considered) |
| | Cap (without | | |
| | desiccant): | | |
| | 02827D-3000 | | |
| | Desiccant: 2 * 1 g | | |
| | silica gel Minipax | | |
| | OR | | |
| | Cap (with inserted | | |
| | desiccant): | | |
| | 02827T-300T | | |
| Gerresheimer/ | Bottle: | 1.04 h (1 week | 12 (1 week |
| DUMA ™ with 2 × 1 g | 035030-3000 | dissolution data | dissolution data) |
| Minipax Intelisorb ™ | 30 ml bottle | considered) | |
| | Cap: | | |
| | 02827D-3000 | | |
| | Desiccant: | | |
| | Intelisorb - 1.0 g | | |
| | IntelliSorb ® MR- | | |
| | 20 | | |

### Measurement of RH by T/RH Probes

As shown in FIG. 10:
The relative humidity at 40° C./75% RH decreases rapidly in bottles with silica gel desiccant down to only a few % with a minor increase over one month.
In bottles with Intellisorb™ desiccants, the relative humidity is much higher and increases slightly over time.
In sachets, the relative humidity equilibrates at a value close to 40% over 6 months.
In bottles without desiccant, there is a progressive increase in relative humidity over time.

### Correlation Between Relative Humidity and Dissolution Profile Stability

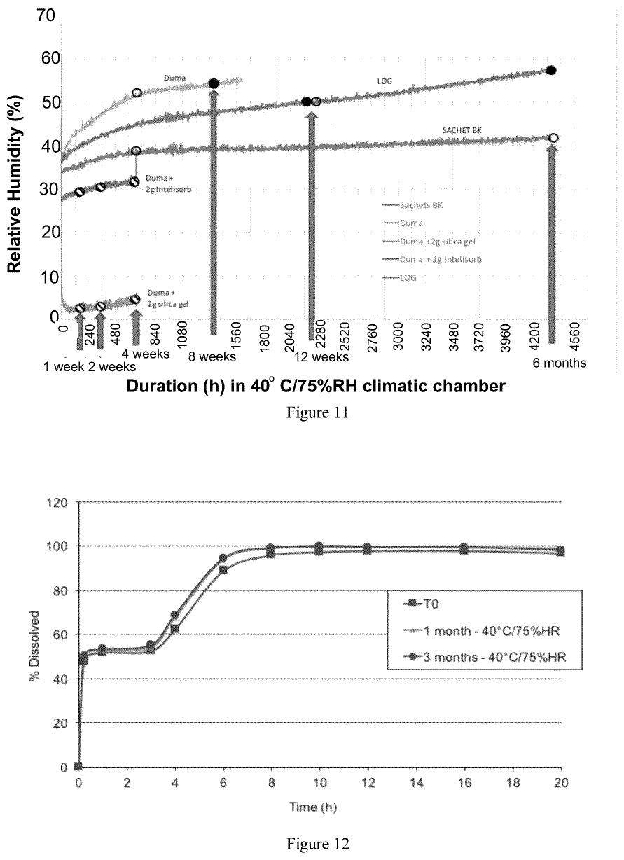

Conclusions regarding dissolution profile stability are plotted on the relative humidity values from FIG. 10 in FIG. 11.
Clear circles indicate time points at which the dissolution profile of the drug product is considered stable, using the criteria in Example 2.
Hatched circles indicate time points at which the dissolution profile of the drug product is considered unstable due to a slowdown of the dissolution profile.
Filled black circles indicate time points at which the dissolution profile of the drug product is unstable due to an acceleration of the dissolution profile.

For the LOG bottles, while an acceleration of the dissolution profile occurred after 3 months (FIG. 6), such an acceleration was not observed during the first stability study (FIG. 5).

Based on this analysis, it can be concluded under the conditions tested that the dissolution profile is unstable, and the packaging is not suitable, if:
after one week at 40° C./75% RH, RH is below 29%; or
before 2 two months at 40° C./75% RH, RH is higher than 54%

### Example 5: Correlation Between Water Vapor Permeability of Packaging and Stability of Dissolution Profile

Based on earlier studies demonstrating dissolution profile stability in some packages and instability in others, a decision was made to investigate the water vapor permeability of these packaging types and to correlate water vapor permeability with the stability of the drug's dissolution profile. We confirmed that:
If the packaging is impermeable enough, the dissolution profile remains stable and no desiccant is needed. Conversely,
If the water vapor transmission rate is too high, some water ingress occurs leading to an acceleration of the dissolution profile (in absence of desiccant).

Table 5 summarizes the water vapor transmission rates of the different package types investigated, and the stability of the dissolution profile in these packages as reported in Example 4. The data is based on a mixture of manufacturer information and applicant testing, and some limited assumptions based on comparability of packaging types. Test results are based on testing under USP 38<671>, or are expected to be produced by testing under USP 38<671>.

TABLE 5

| | | Water Vapor | Stability* of |
|---|---|---|---|
| Supplier | Item | Transmission Rate | Dissolution profile |
| Gerresheimer/ | Bottle: | 6.0-8.9 mg/day/l | Unstable |
| DUMA ™ w/o | 035030-3000 | average = 7.0 mg/d/l | |
| desiccant | 30 ml bottle | | |
| | Cap: | | |
| | 02827D-3000 | | |
| Log ™ | Bottle: H2OO2: | average = 1 mg/d/l | Stable |
| | 40 ml White Bot. | | |
| | 33/400 MBF 20 | | |
| | Cap: 33 mm White | | |
| | CR Cap + IHS | | |
| | liner | | |
| Bischof & Klein | PET/ALU/PE | average = 0.6 mg/d/l | Stable |
| (sachets) | with 9 μm ALU foil | | |
| Constantia (stick- | PET/adhesive | average = 0.3 mg/d/l | Stable |
| pack) | layer/ALU/ | | |
| | copolymer with | | |
| | 12 μm ALU foil | | |

*A packaged formulation was judged stable if, after 2 months at 40° C./75% RH, the absolute value (t’-t₀) was less than 0.83 h (=50 min) and/or the difference in active ingredient dissolved at all dissolution sampling times was less than 10% as explained in Example 2.

### Example 6: Relationship Between Coating Composition, Packaging/RH, and Stability of Dissolution Profile

The stability of alternative formulations was also investigated at 40° C./75% RH, using the method reported in Example 2:
in Gerresheimer DUMA™ bottles with desiccant (Bottle: 035030-3000 30 ml bottle/Cap (with inserted 2 g silica gel desiccant): 02827T-300T over 2 months to determine if a slowdown of the dissolution profile occurs; and
in Bischof & Klein sachets to check dissolution profile stability over 3 months.

Dissolution profiles were assessed for:
IR/MR oxybate formulations with MR composition comprising pH dependent polymers Eudragit™ L100-55/Eudragit™ S100 but with a different ratio compared to the formulations described in Example 1
IR/MR oxybate formulations with MR composition comprising a Lubritab™ amount in the coating other than 60% Lubritab™;
IR/MR oxybate formulations with an amount of IR oxybate lower than 50% of the dose.

Table 6 reports the results of the foregoing testing:

TABLE 6

| | | | |
|---|---|---|---|
| | | | Max of |
| Formulation composition | | | Δ (% API |
| differences compared to | | t’-t₀ | dissolved) as |
| formulations described in | | as described | described in |
| Example 1 | Packaging | in Example 2 | Example 2 |

Reference formulation: second formulation in Ex 1

| | | | |
|---|---|---|---|
| MR coating composition: | Bottle with | 1.5 h (1 | 13% (one |
| 60% Lubritab/40% | desiccant | month | month |
| [Eudragit L100-55/S100 | | dissolution | dissolution |
| 1:14] | | data | data |
| MR coating represents | | considered) | considered) |
| 25% by weight of the total | | | |
| weight of the MR particles | | | |
| of gamma- | | | |
| hydroxybutyrate | | | |

Reference formulation: first formulation in Ex 1

| | | | |
|---|---|---|---|
| MR coating composition: | Sachet | −0.05 h | 3% |
| 60% Lubritab/40% | | (3 months | (3 months |
| [Eudragit L100-55/S100 | | dissolution | dissolution |
| 1:14] | | data | data |
| MR coating represents | | considered) | considered) |
| 25% by weight of the total | Bottle with | 0.59 h (1 | 14% (1 |
| weight of the MR particles | desiccant | month | month |
| of gamma- | | dissolution | dissolution |
| hydroxybutyrate | | data | data |
| | | considered) | considered) |
| MR coating composition: | Sachet | 0.20 h | 3% |
| 60% Lubritab/40% | | (3 months | (3 months |
| [Eudragit L100-55/S100 | | dissolution | dissolution |
| 1:0.2] | | data | data |
| | | considered) | considered) |
| | Bottle with | 1.40 h (1 | 14% (1 |
| | desiccant | month | month |
| | | dissolution | dissolution |
| | | data | data |
| | | considered) | considered) |
| MR coating composition: | Sachet | 0.15 h | 6% |
| 60% Lubritab/40% | | (3 months | (3 months |
| [Eudragit L100-55/S100 | | dissolution | dissolution |
| 1:1.14] | | data | data |
| MR microparticles | | considered) | considered) |
| represent 70% of | Bottle with | 1.10 h (1 | 20% (1 |
| the dose | desiccant | month | month |
| | | dissolution | dissolution |
| | | data | data |
| | | considered) | considered) |
| MR coating composition: | Sachet | −0.55 h | 7% |
| 40% Lubritab/60% | | (3 months | (3 months |
| [Eudragit L100-55/S100 | | dissolution | dissolution |
| 1:2] | | data | data |
| MR coating represents | | considered) | considered) |
| 40% by weight of the total | Bottle with | 2.57 h (1 | 34% (1 |
| weight of the MR particles | desiccant | month | month |
| of gamma- | | dissolution | dissolution |
| hydroxybutyrate | | data | data |
| | | considered) | considered) |
| MR coating composition: | Sachet | −0.13 h | 5% |
| 70% Lubritab/30% | | (3 months | (3 months |
| [Eudragit L100-55/S100 | | dissolution | dissolution |
| 1:2] | | data | data |
| MR coating represents | | considered) | considered) |
| 25% by weight of the total | Bottle with | 1.30 h | 12% |
| weight of the MR particles | desiccant | (3 months | (3 months |
| of gamma- | | dissolution | dissolution |
| hydroxybutyrate | | data | data |
| | | considered) | considered) |

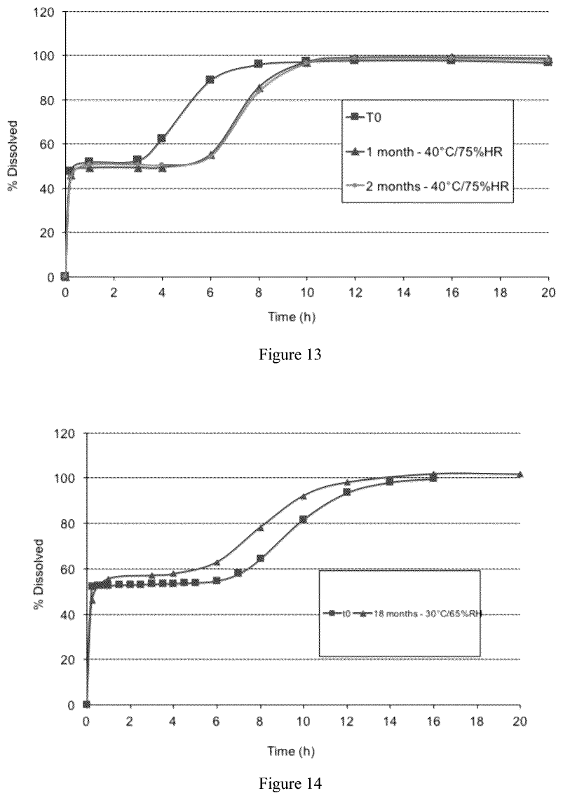

The dissolution profiles of the composition comprising MR coated microparticles with 40% Lubritab in the coating are illustrated respectively in FIGS. 12 and 13 for the composition packaged in sachets and bottles with desiccant. All the packaged compositions in sachets were stable, when evaluated by the criteria in Example 2, whereas none was stable in bottles with desiccant.

### Example 7: Chemical and Dissolution Profile Stability Based on Packaging Type

The chemical stability and dissolution profile stability for the first formulation of example 1 was investigated using various packaging types during three stability studies conducted at 30° C./65% RH:
DUMA™ bottle without desiccant
DUMA™ bottle with 2 g silica gel desiccant in cap
REXAM™ bottle heat sealed without desiccant (REXAM 30410 HDPE Blanc 60 ml/Cap: REXAM 28/400 FG PP BLANC Word FS M-1 Liner)

Each of the experiments evaluated the stability of a 4.50 g dose of the formulation. The initial formulation water content was 1.2%. Initial degradants were less than 0.05%. The results of the chemical stability testing are reported in Table 7a for the 30° C./65% RH condition:

TABLE 7a

| | T₀ | | | T_{12 months} | | | T_{18 months} | | |
|---|---|---|---|---|---|---|---|---|---|
| | Dose | Deg % | % water | Dose | Deg % | % water | Dose | Deg % | % water |
| DUMA ™ | 4.52 g | <0.05 | 1.2 | 4.11 g | 0.94 | 2.5 | 4.27 g | 1.17 | 3.8 |
| bottle without | | | | | | | | | |
| desiccant | | | | | | | | | |
| DUMA ™ | | | | 4.49 g | <0.05 | 0.9 | 4.49 g | <0.05 | 0.9 |
| bottle with 2 g | | | | | | | | | |
| silica gel | | | | | | | | | |
| desiccant in | | | | | | | | | |
| cap | | | | | | | | | |
| REXAM ™ | | | | 4.19 g | 0.79 | 2.5 | 4.15 g | 1.04 | 3.9 |
| bottle heat | | | | | | | | | |
| sealed | | | | | | | | | |
| without | | | | | | | | | |
| desiccant | | | | | | | | | |

As can be seen, the only packaged formulation that remained chemically stable in these experiments was the formulation in bottles with desiccant, when the relative humidity inside the bottle was kept the lowest.

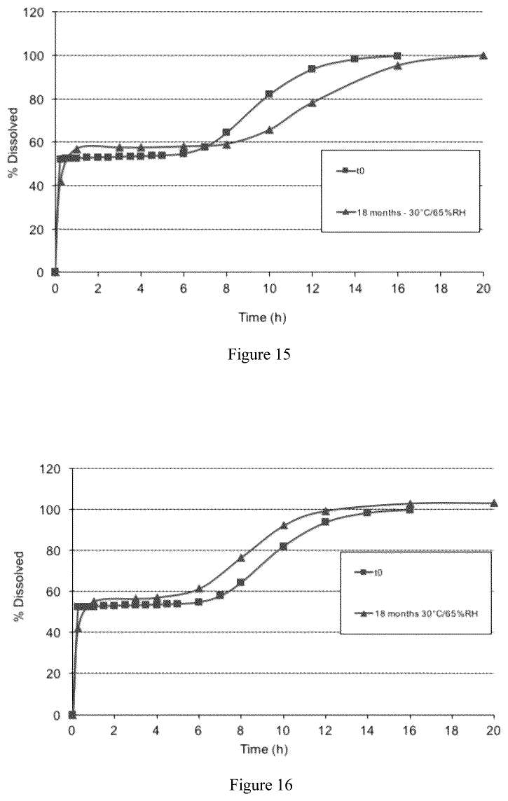

Results of the dissolution testing are depicted in FIGS. 14, 15, and 16, where one can observe:
An acceleration of the dissolution profile in the DUMA™ bottle without desiccant (FIG. 14);
A slowing of the dissolution profile for the DUMA™ bottle with desiccant in cap (FIG. 15); and
An acceleration of the dissolution profile for the REXAM™ bottle heat sealed without desiccant (FIG. 16).

None of the three packaged compositions has a stable dissolution profile after 18 months at 30° C./65% RH according to dissolution stability criteria described in example 2, as illustrated in Table 7b.

TABLE 7b

| | | | Max of |
|---|---|---|---|
| | | | Δ (% API |
| Supplier | | t’-t₀ | dissolved) as |
| (type of | Packaging | as described | described in |
| packaging) | references | in example 2 | example 2 |
| Gerresheimer/ | Bottle: | −2.1 h | 14 |
| DUMA ™ w/o | 035030-3000 | | |
| desiccant (bottles) | 30 ml bottle | | |
| | Cap: | | |
| | 02827D-3000 | | |
| Gerresheimer/ | Bottle: | 1.13 h | 16 |
| DUMA ™ with 2 g | 035030-3000 | | |
| desiccant (bottles) | 30 ml bottle | | |
| | Cap (with inserted | | |
| | desiccant): | | |
| | 02827T-300T | | |
| Rexam/REXAM ™ | Bottle: 30410 | −1.8 h | 12 |
| bottle heat sealed | HDPE Blanc 60 ml/ | | |
| w/o desiccant | Cap: 28/400 FG | | |
| | PP BLANC Word | | |
| | FS M-1 Liner) | | |

The stability of the first formulation of example 1 was previously investigated during two stability studies conducted at 40° C./75% RH in 2 of the 3 packages described above:
DUMA™ bottle without desiccant
DUMA™ bottle with desiccant in cap The dissolution profile stability of the 2 packaged compositions has already been discussed in example 4 (cf FIG. 7 for the DUMA™ bottle without desiccant and FIG. 8 DUMA™ bottle with desiccant in cap). Neither of the two packaged compositions has a stable dissolution profile after 2 months at 40° C./75% RH.

In parallel with the dissolution profile assessment, the chemical stability of the packaged formulations was also evaluated. In the DUMA™ bottle without desiccant, the amount of degradant formed after 2 months at 40° C./75% RH was 0.4%. In the DUMA™ bottle with desiccant in the cap, the amount of degradant formed after 2 months at 40° C./75% RH was less than 0.05%.

The chemical stability and dissolution profile stability for the first formulation of example 1 was also investigated in Bischof & Klein PET/ALU/PE sachets with 9 μm ALU foil for a 4.50 g dose of the formulation. The initial formulation water content was 1.0%. Initial degradants were less than 0.05%. After 18 months at 30° C./65% RH, the formulation water content is equal to 0.8% and the amount of degradation products is 0.1%.

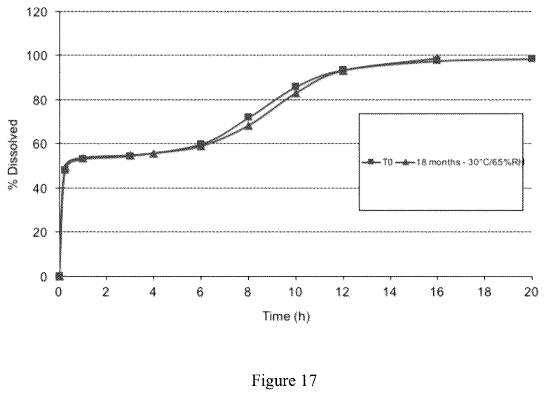

Results of the dissolution testing are depicted in FIG. 17 where one can observe that the packaged composition has a stable dissolution profile after 18 months at 30° C./65% RH according to dissolution criteria described in Example 2.

TABLE 7c

| | | | Max of |
|---|---|---|---|
| | | | Δ(% API |
| Supplier | | t′-t₀ | dissolved) as |
| (type of | Packaging | as described | described in |
| packaging) | references | in Example 2 | example E* |
| Bischof & Klein | PET/ALU/PE | 0.65 h | 4 |
| (sachets) | with 9 μm ALU foil | | |

### Example 8. In Vivo Pharmacokinetic Study of Second Formulation According to Example 1

Pharmacokinetic testing was undertaken in vivo in healthy human volunteers according to the principles described in FDA's March 2003 Guidance for Industry on BIOAVAILABILITY AND BIOEQUIVALENCE STUDIES FOR ORALLY ADMINISTERED DRUG PRODUCTS—GENERAL CONSIDERATIONS. All testing was performed in subjects two hours after eating a standardized dinner. XYREM® doses were administered in two equipotent doses four hours apart. All other tested doses were manufactured as described in the second formulation of Example 1. The standardized dinner consisted of 25.5% fat, 19.6% protein, and 54.9% carbohydrates.

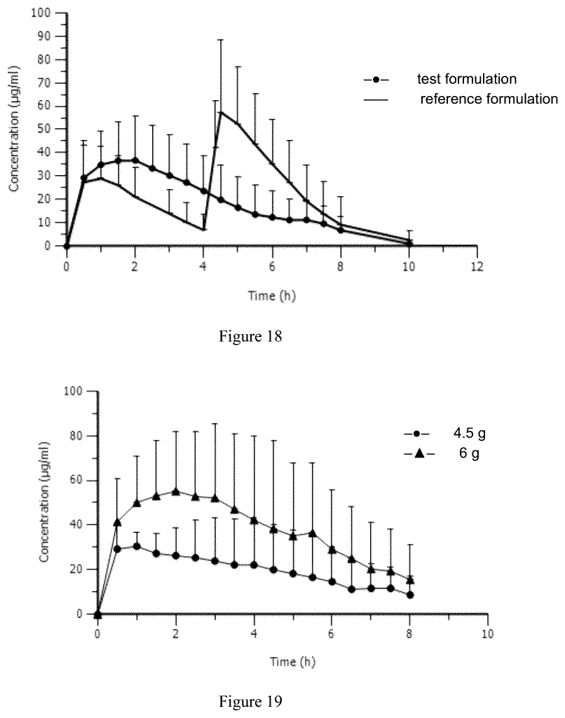

The second formulation of Example 1 given as a 4.5 g once-nightly dose rather than a standard XYREM® dosing twice (2×2.25 g) nightly 4 hours apart, produced a dramatically different pharmacokinetic profile than XYREM® as shown in FIG. 18. As summarized below (Tables 8a and 8b), 4.5 g nighttime doses of finished composition of the invention equivalent to twice-nightly doses of XYREM® (2×2.25 g) provided somewhat less total exposure to sodium oxybate with a later median Tₘₐₓ than the initial XYREM® dose. The relative bioavailability was about 88%. Composition according to the invention avoids the high second-dose peak concentration of XYREM® and therefore does not exhibit the substantial between-dose fluctuations in concentration, while achieving a comparable mean C₈ₕ.

TABLE 8a
Pharmacokinetic Parameters of finished composition
of second formulation vs. XYREM ®

| | Mean | Mean | Median Tmax |
|---|---|---|---|
| | Cmax | AUCinf | (hour) |
| | (μg/mL) (% CV) | (h*μg/mL) | (min-max) |
| Second | 44.35 (38) | 188.88 (44) | 1.5 (0.5-4) |
| formulation | | | |
| 4.5 g | | | |
| XYREM ® | 1st dose: 33.41 | 214.32 (48) | 1st dose: 1.00 (0.5-2) |
| 2 × 2.25 g | (41) | | 2nd dose: 4.50 (4.33-6.5) |
| | 2nd dose: 65.91 | | |
| | (40) | | |

TABLE 8b
Mean plasma concentration of gamma-hydroxybutyrate (microgram/mL)
versus time of second formulation and XYREM ®

| | Second | Second | | |
|---|---|---|---|---|
| | formulation | formulation | Second | |
| | 4.5 g | 6.0 g | formulation | XYREM ® |
| | (2 h after meal) | (2 h after meal) | 7.5 g | (2 × 2.25 g) |
| Time | pooled mean | pooled mean | (2 h after meal) | part I |
| (hour) | (N = 26) | (N = 19) | (N = 11) | (N = 15) |
| 0 | 0.00 | 0.00 | 0.00 | 0.00 |
| 0.5 | 29.31 | 36.44 | 43.19 | 27.44 |
| 1 | 34.93 | 49.97 | 63.32 | 28.97 |
| 1.5 | 36.63 | 54.66 | 73.40 | 26.12 |
| 2 | 36.78 | 54.82 | 67.96 | 21.11 |
| 2.5 | 33.35 | 53.05 | 66.59 | NA |
| 3 | 30.28 | 50.25 | 62.13 | 13.93 |
| 3.5 | 27.30 | 47.22 | 59.45 | 10.25 |
| 4 | 23.66 | 43.06 | 57.40 | 6.92 |
| 4.5 | 19.89 | 39.13 | 50.85 | 57.33 |
| 5 | 16.55 | 34.28 | 45.09 | 52.27 |
| 5.5 | 13.62 | 32.11 | 44.94 | 43.55 |
| 6 | 12.40 | 25.84 | 42.36 | 35.20 |
| 6.5 | 11.25 | 22.36 | 41.02 | 27.44 |
| 7 | 11.27 | 18.07 | 40.76 | 19.36 |
| 7.5 | 9.65 | 15.41 | 35.83 | 13.88 |
| 8 | 6.86 | 12.80 | 30.94 | 9.24 |
| 10 | 1.08 | 2.38 | 7.99 | 2.64 |
| 12 | NC | 0.52 | 1.47 | NC |

NC: Not Calculated

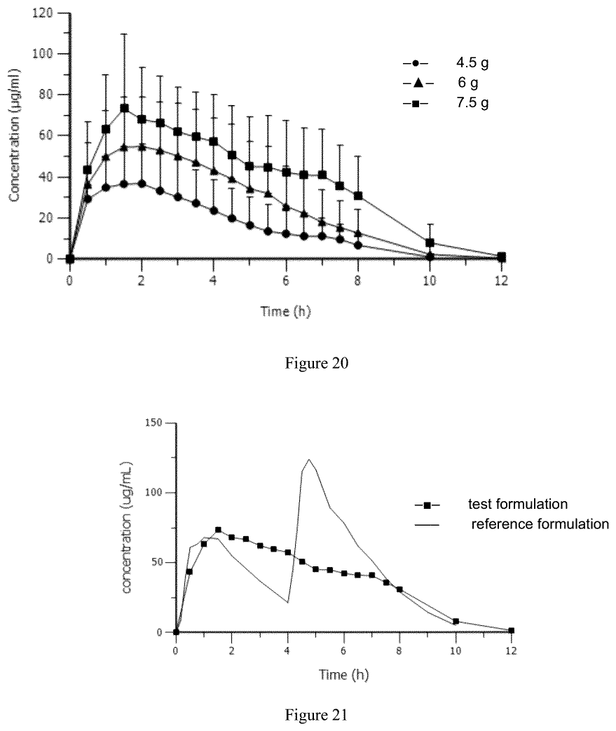

The pharmacokinetic profile of a single 6 g dose of the second formulation was also tested and found to have a similar pharmacokinetic profile as the 4.5 g dose. FIG. 19 provides a pharmacokinetic profile comparison of a single 4.5 g or 6 g dose of the second formulation in the same 7 subjects. The pharmacokinetic profile for a 7.5 g dose of the second formulation was also obtained. FIG. 20 and Table 8c provide data on a single 4.5 g, 6 g and 7.5 g dose, showing effects on Tmax, Cmax, C8h, AUC8h and AUCinf related to dose strength. The 7.5 g dose achieved a mean C8h equal to about 31 microgram/mL which represents approximately 128.5% of the C8h obtained for XYREM® dosed 2×3.75 g which was extrapolated to be approximately 24.07 microgram/mL from published data. The 7.5 g dose achieved a ratio of AUC8 h to AUCinf of about 0.89, whereas the ratio was 0.83 and 0.93 for the 4.5 g and 6 g doses respectively.

TABLE 8c
Pharmacokinetic Parameters of 4.5 g, 6 g, and 7.5 g of second formulation

| Second | Mean | Mean | Mean | Median | | Mean |
|---|---|---|---|---|---|---|
| formulation | Cₘₐₓ | AUC_{inf} | AUC_{8 h} | Tₘₐₓ | | C_{8 h} |
| 4.5 g | 44.35 (38) | 188.88 (47) | 174.68 (48) | 1.5 | (0.5-4) | 6.86 (84) |
| 6 g | 65.46 (35) | 307.34 (48) | 290.97 (47) | 3 | (0.5-5.5) | 12.8 (82) |
| 7.5 g | 88.21 (30) | 454.99 (34) | 404.88 (31) | 2 | (0.5-6) | 30.94 (34) |

FIG. 21 and table 8d compare the pharmacokinetic parameters AUC_{inf} and C₈ₕ obtained for 7.5 g of the second formulation to the same parameters calculated for 2×4.5 g, i.e. 9 g total dose of XYREM®. The data show that a 7.5 g dose of a formulation according to the invention given once nightly exhibits a similar PK profile to 9 g of XYREM® given in two separate equal doses.

TABLE 8d
Pharmacokinetic Parameters of 7.5 g of second formulation
compared to 2 × 4.5 g of XYREM ®

| | Mean | Mean | Ratio (%) AUC_{inf} | Ratio (%) C_{8 h} |
|---|---|---|---|---|
| | C_{8 h} | AUC_{inf} | composition to | composition to |
| | (μg/mL) | (μg/mL*h) | AUC_{inf} Xyrem ® | C_{8 h} Xyrem ® |
| XYREM ® | 28.9 | 518 | NA | NA |
| 2 × 4.5 g | | | | |
| Second | 30.9 | 455 | 88% | 107% |
| Formulation | | | | |
| 7.5 g | | | | |

### Example 9. In Vivo Comparison of Two Different Batch Sizes of First Formulation According to Example 1

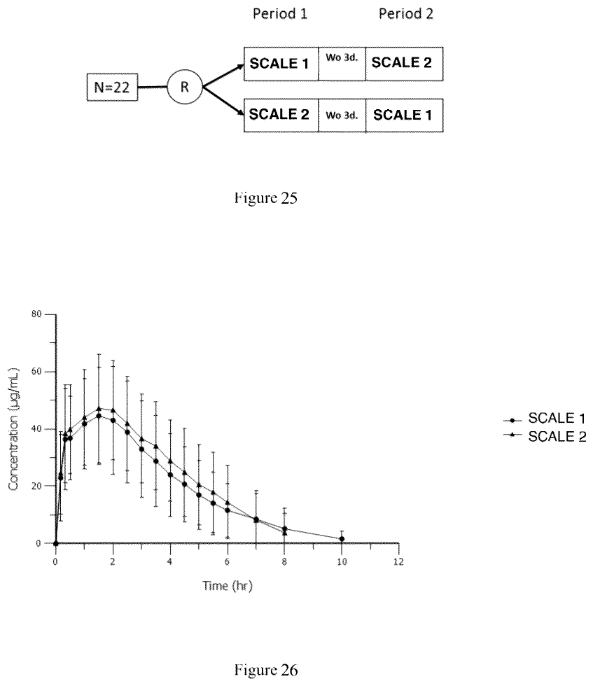

A comparative, open-label, randomized, single-dose, crossover study was performed to evaluate 2 different batch sizes (scale 1 and scale 2) of the first formulation manufactured as described in Example 1, at a dose of 4.5 g administered two hours post-evening meal in healthy volunteers. 22 subjects were randomized to a treatment sequence in a 1:1 ratio and were allocated to one of the following treatment sequences, as depicted in FIG. 25:
4.5 g of FT218 batch scale 1 (period 1) followed by 4.5 g of FT218 batch scale 2 (period 2) or
4.5 g of FT218 batch scale 2 (period 1) followed by 4.5 g of FT218 batch scale 1 (period 2)

There was a wash-out of a least 3 days between drug administrations. 22 and 21 healthy volunteers received scale 1 and scale 2 batches respectively (one subject stopped the study after the 1st period and received only FT218 scale 1). Blood sampling for pharmacokinetics of sodium oxybate in plasma were taken each period at pre-dose, 10 and 20 minutes post-dose, and 0.5, 1, 1.5, 2, 2.5, 3, 3.5, 4, 4.5, 5, 5.5, 6, 7, 8, 10, 12 and 14 hours post-dose PK parameters were calculated using non-compartmental analysis from the plasma concentration-time data for sodium oxybate.

The data shown below in Tables 9a and 9b (mean PK parameters and plasma concentration), as well as FIG. 26, demonstrates that scale 1 and scale 2 formulations according to the invention exhibit a similar PK profile.

TABLE 9a
Mean PK Parameters

| | Mean | Mean | Mean | Median | Mean |
|---|---|---|---|---|---|
| | Cₘₐₓ | AUC_{inf} | AUC_{0-8 h} | Tₘₐₓ | C_{8 h} |
| | (μg/mL) | (μg/mL*h) | (μg/mL*h) | (hour) | (μg/mL) |
| FT218 | (% CV) | (% CV) | (% CV) | (min-max) | (% CV) |
| 4.5 g | 47.9 | 200 | 194 | 1.5 | 5.1 |
| scale 1 | (37) | (45) | (44) | (0.33-3.5) | (140) |
| 4.5 g | 52.5 | 219 | 215 | 1.5 | 3.7 |
| scale 2 | (32) | (44) | (42) | (0.33-4.5) | (186) |

TABLE 9b
Mean plasma concentrations (microgram/mL)

| | scale 1 4.5 g | scale 2 4.5 g |
|---|---|---|
| | (2 h after meal) | (2 h after meal) |
| Time (hr) | (N = 22) | (N = 21) |
| 0 | 0 | 0 |
| 0.17 | 22.9 | 24.6 |
| 0.33 | 36.3 | 38.3 |
| 0.5 | 36.7 | 39.8 |
| 1 | 41.7 | 44.0 |
| 1.5 | 44.5 | 47.2 |
| 2 | 43.0 | 46.6 |
| 2.5 | 38.9 | 41.9 |
| 3 | 32.9 | 36.6 |
| 3.5 | 28.7 | 34.0 |
| 4 | 23.9 | 28.8 |
| 4.5 | 20.7 | 24.8 |
| 5 | 16.9 | 20.6 |
| 5.5 | 14.0 | 17.9 |
| 6 | 11.5 | 14.4 |
| 7 | 8.5 | 8.2 |
| 8 | 5.1 | 3.7 |
| 10 | 1.6 | NC |
| 12 | NC | NC |
| 14 | NC | NC |

NC: Not Calculated.

### Example 10: Storage Stability Testing of 4.5 g Batches of First Formulation According to Example 1

A study was performed to evaluate the stability of multiple batches of the first formulation manufactured as described in Example 1, at a dose of 4.5 g after storage for up to 72 months. The stability was measured after storage at a range of temperatures (25° C., 30° C., 40° C.) and at a range of relative humidities (RH) (60% RH, 65% RH, 75% RH).

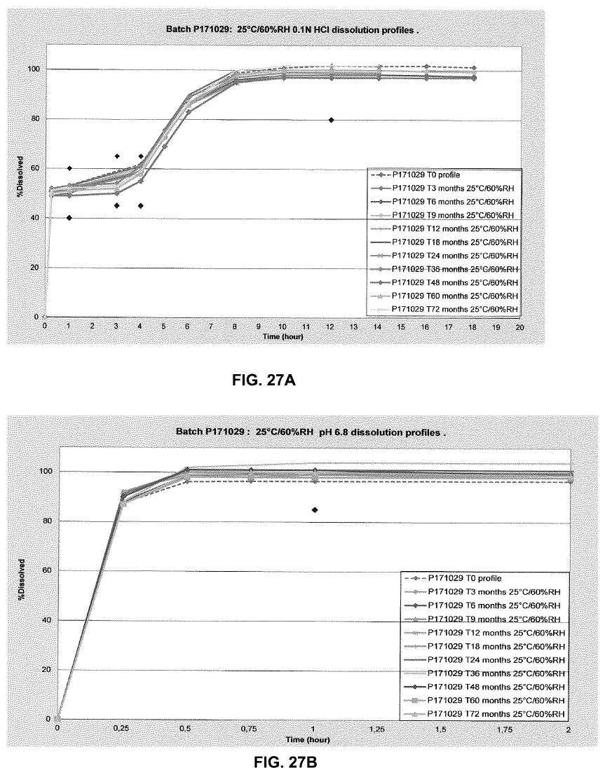

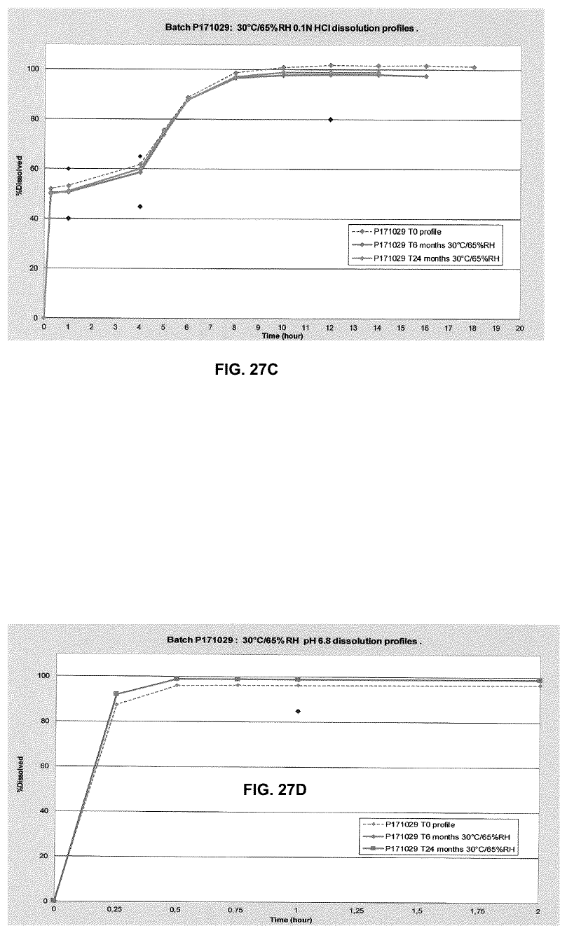

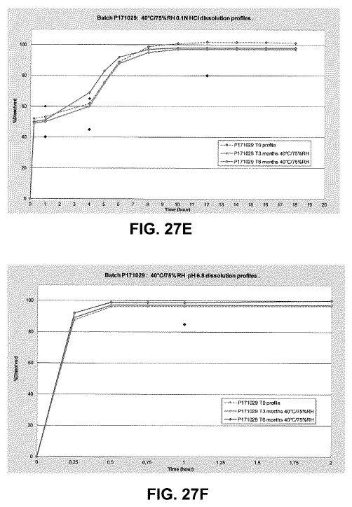

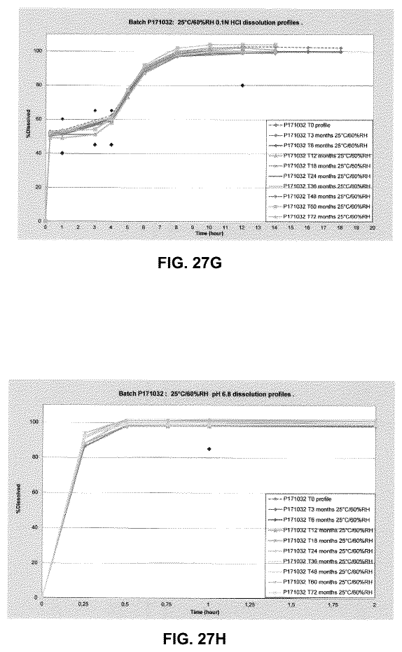

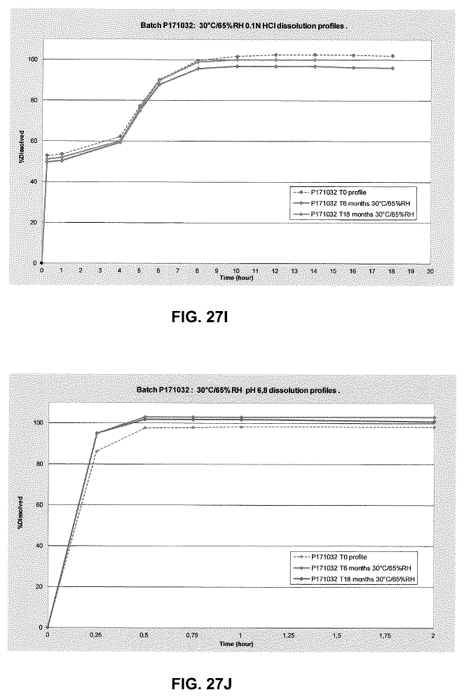

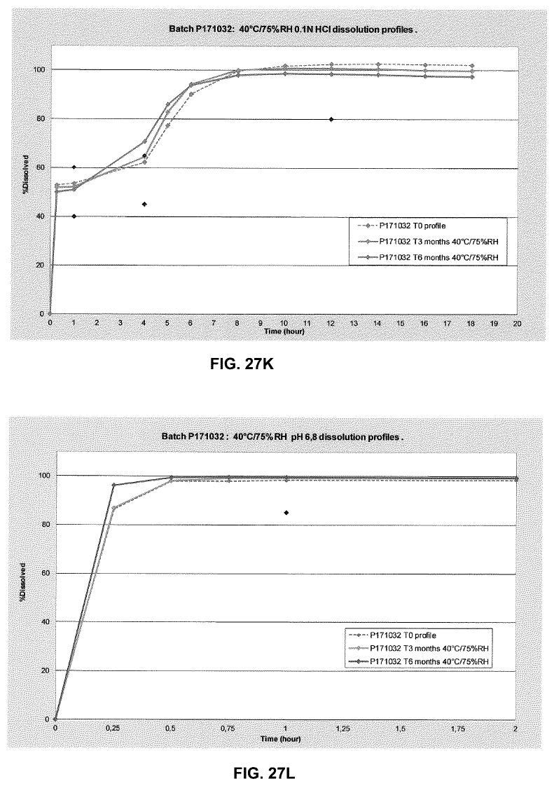

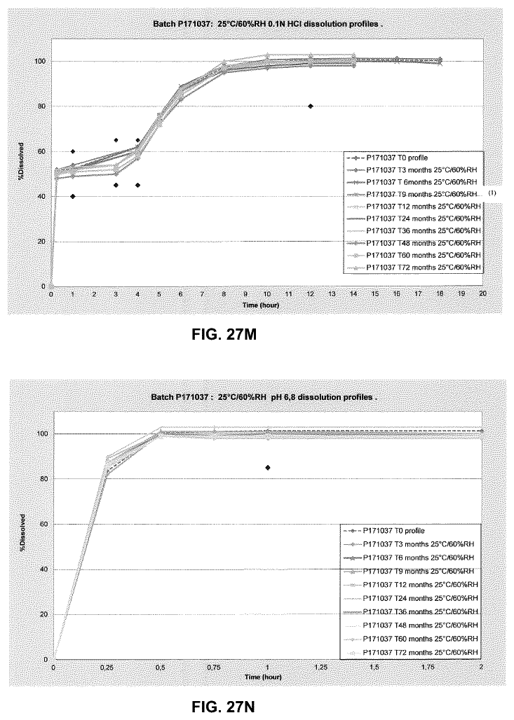

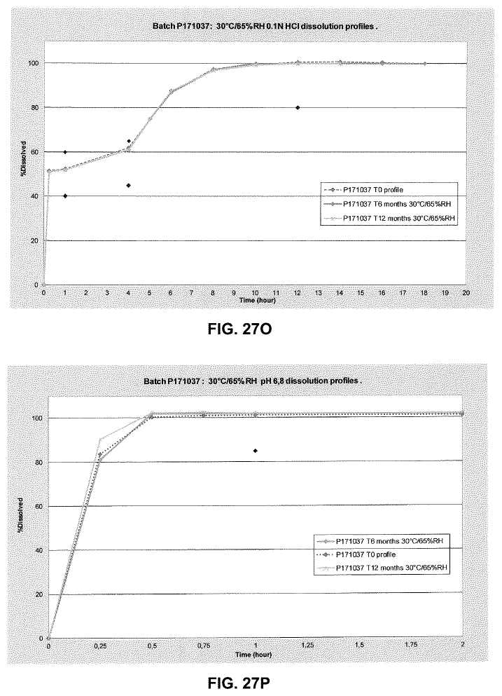

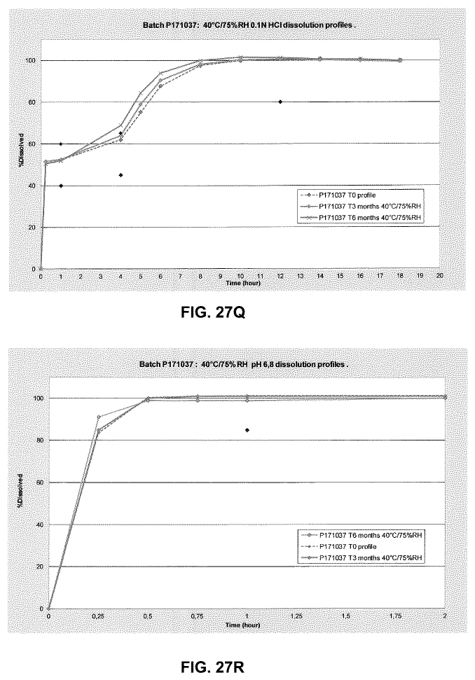

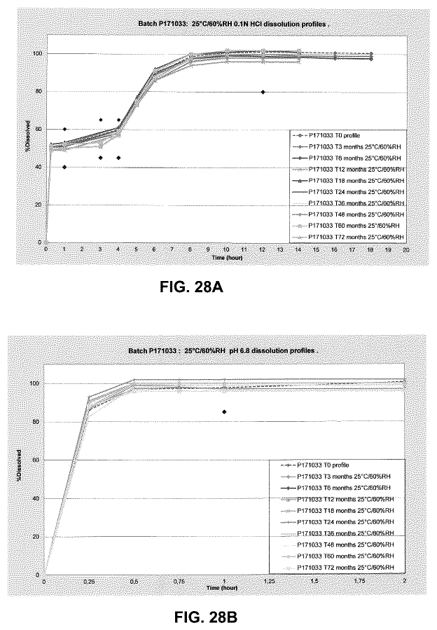

Tables 10a-10c include averages of pH after reconstitution, amount of sodium oxybate by HPLC assay, related substances by HPLC (% w/w), water content, and dissolution of multiple batches of the 4.5 g dose of the formulation. FIGS. 27A-27R show results for individual batches of the 4.5 g dose of the formulation. The data shown below in Tables 10a-10c and FIGS. 27A-27R show that the 4.5 g formulation is stable and suitable for administration when stored up to at least 72 months at 25° C./60% RH, up to at least 24 months when stored at 30° C./65% RH, and up to at least 6 months at 40° C./75% RH.

TABLE 10a
4.5 g dose stored at 25° C./60% RH

| Stability time point (months) | T0 | T3 | T6 | T9 | T12 | T18 | T24 | T36 | T48 | T60 | T72 |
|---|---|---|---|---|---|---|---|---|---|---|---|
| pH after reconstitution: | | | | | | | | | | | |
| ≤5.9 | 5.8 | 5.8 | 5.7 | 5.7 | 5.8 | 5.7 | 5.7 | 5.7 | 5.6 | 5.7 | 5.7 |
| Sodium oxybate HPLC Assay: | | | | | | | | | | | |
| 4.05 to 4.95 g/unit | 4.52 | 4.51 | 4.49 | 4.54 | 4.49 | 4.55 | 4.53 | 4.62 | 4.51 | 4.58 | 4.52 |
| 90.0 to 110.0% of label claim | 100.5 | 100.2 | 99.8 | 100.9 | 99.7 | 101.2 | 103.7 | 102.6 | 100.3 | 100.7 | 100.5 |
| Related substances by HPLC (% | | | | | | | | | | | |
| w/w): | | | | | | | | | | | |
| Gamma-butyrolactone: ≤0.5% w/w | ≤0.05 | ≤0.05 | ≤0.05 | ≤0.05 | ≤0.05 | ≤0.05 | ≤0.06 | 0.07 | 0.08 | 0.11 | 0.12 |
| Individual unspecified impurity: | ND | ND | ND | ND | ND | ND | ND | ND | ND | ND | ND |
| ≤0.10% w/w | | | | | | | | | | | |
| Total related substances: ≤0.5% | ≤0.05 | ≤0.05 | ≤0.05 | ≤0.05 | ≤0.05 | ≤0.05 | ≤0.05 | 0.1 | 0.1 | 0.1 | 0.1 |
| w/w | | | | | | | | | | | |
| Water content: | | | | | | | | | | | |
| ≤4.0% w/w | 0.40 | 0.70 | 0.67 | 0.60 | 0.60 | 0.55 | 0.67 | 0.63 | 0.57 | 0.53 | 0.60 |
| 0.1N HCl Dissolution test by | | | | | | | | | | | |
| HPLC: | | | | | | | | | | | |
| (% dissolved) | | | | | | | | | | | |
| 40% ≤ T1 hour ≤60% | 53 | 52 | 52 | 52 | 52 | 53 | 51 | 53 | 50 | 52 | 51 |
| 45% ≤ T3 hours ≤65% | | | | | | | | 54 | 50 | 53 | 53 |
| 45% ≤ T4 hour ≤65% | 62 | 60 | 60 | 60 | 60 | 60 | 59 | 61 | 57 | 59 | 59 |
| T12 hours ≥ 80% | 102 | 99 | 99 | 100 | 100 | 101 | 99 | 101 | 98 | 101 | 102 |
| pH 6.8 dissolution test by HPLC: | | | | | | | | | | | |
| (% dissolved) | | | | | | | | | | | |
| T1 hour ≥ 85% | 98 | 99 | 99 | 100 | 99 | 101 | 102 | 102 | 102 | 99 | 99 |

TABLE 10b
4.5 g dose stored at 30° C./65% RH

| Stability time point (months) | T0 | T6 | T15 | T18 | T24 |
|---|---|---|---|---|---|
| pH after reconstitution: | | | | | |
| ≤5.9 | 5.8 | 5.7 | 5.8 | 5.7 | 5.7 |
| Sodium oxybate HPLC Assay: | | | | | |
| 4.05 to 4.95 g/unit | 4.50 | 4.51 | 4.51 | 4.61 | 4.62 |
| 90.0 to 110.0% of label claim | 99.9 | 100.2 | 100.2 | 102.4 | 102.7 |
| Related substances by HPLC (% w/w): | | | | | |
| Gamma-butyrolactone: ≤0.5% w/w | ≤0.05 | ≤0.05 | ≤0.07 | ≤0.08 | ≤0.1 |
| Individual unspecified impurity: ≤0.10% | ND | ND | ND | ND | ND |
| w/w | | | | | |
| Total related substances: ≤0.5% w/w | ≤0.05 | ≤0.05 | ≤0.07 | ≤0.08 | 0.1 |
| Water content: | | | | | |
| ≤4.0% w/w | 0.47 | 0.47 | 0.8 | 0.8 | 0.6 |
| 0.1N HCl Dissolution test by HPLC | | | | | |
| (% dissolved) | | | | | |
| 40% ≤ T1 hour ≤ 60% | 53 | 51 | 52 | 52 | 51 |
| 45% ≤ T4 hour ≤ 65% | 62 | 60 | 61 | 60 | 60 |
| T12 hours ≥ 80% | 102 | 98.3 | 100 | 100 | 99 |
| pH 6.8 dissolution test by HPLC (% | | | | | |
| dissolved) | | | | | |
| T1 hour ≥ 85% | 99 | 101 | 120 | 120 | 100 |

TABLE 10c
4.5 g dose stored at 40° C./75% RH

| Stability time point (months) | T0 | T3 | T6 |
|---|---|---|---|
| pH after reconstitution: | | | |
| ≤5.9 | 5.8 | 5.8 | 537 |
| Sodium oxybate HPLC Assay: | | | |
| 4.05 to 4.95 g/unit | 4.52 | 4.49 | 4.46 |
| 90.0 to 110.0% of label claim | 100.5 | 99.7 | 99.2 |
| Related substances by HPLC (% w/w): | | | |
| Gamma-butyrolactone: ≤0.5% w/w | ≤0.05 | 0.08 | 0.12 |
| Individual unspecified impurity: ≤0.10% | ND | ND | ND |
| w/w | | | |
| Total related substances: ≤0.5% w/w | ≤0.05 | 0.10 | 0.10 |
| Water content: | | | |
| ≤4.0% w/w | 0.40 | 0.73 | 0.9 |
| 0.1N HCl Dissolution test by HPLC | | | |
| (% dissolved) | | | |
| 40% ≤ T1 hour ≤ 60% | 53 | 52 | 51 |
| 45% ≤ T4 hour ≤ 65% | 62 | 63 | 70 |
| T12 hours ≥ 80% | 102 | 99.3 | 99 |
| pH 6.8 dissolution test by HPLC (% | | | |
| dissolved) | | | |
| T1 hour ≥ 85% | 98.3 | 99 | 99.3 |

### Example 11: Storage Stability Testing of 6.0 g Batch of Formulation According to Example 1

A study was performed to evaluate the stability of a batch of the formulation manufactured as described in Example 1, at a dose of 6.0 g after storage for up to 72 months. The stability was measured after storage at a range of temperatures (25° C., 30° C., 40° C.) and at a range of relative humidities (RH) (60% RH, 65% RH, 75% RH).

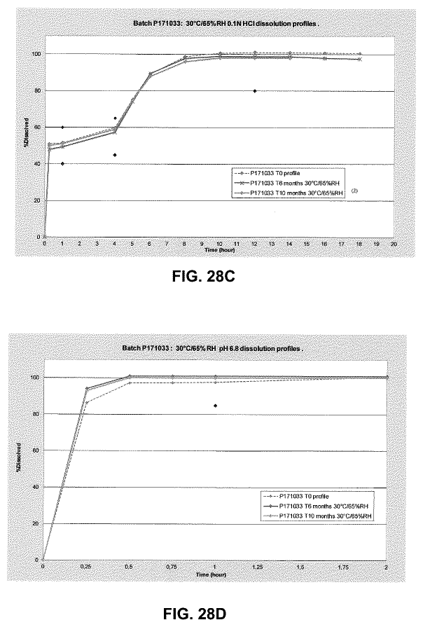

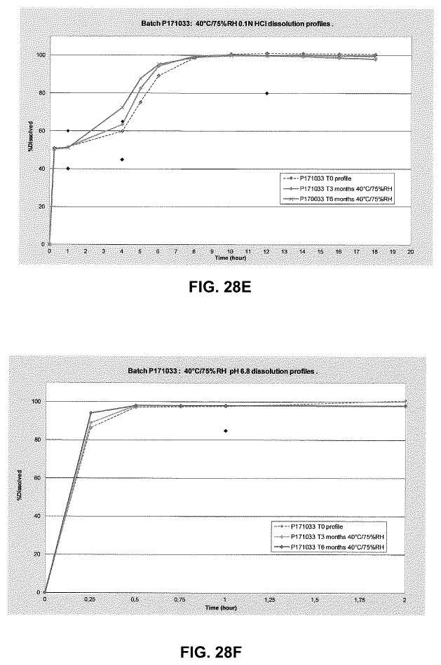

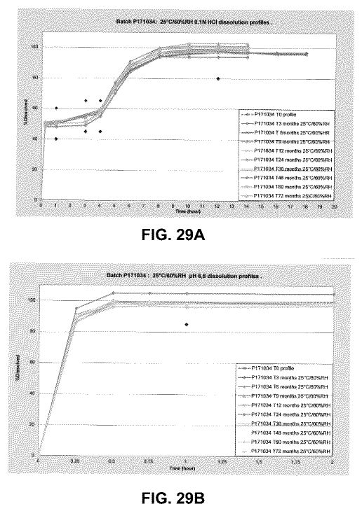

Tables 11a-11c include data for pH after reconstitution, amount of sodium oxybate by HPLC assay, related substances by HPLC (% w/w), water content, and dissolution of a batch of the 6.0 g dose of the formulation. FIGS. 28A-28F show results for a batch of the 6.0 g dose of the formulation. The data shown below in Tables 11a-11c and FIGS. 28A-28F show that the 6.0 g formulation is stable when stored up to at least 72 months at 25° C./60% RH, up to at least 24 months when stored at 30° C./65% RH, and up to at least 6 months at 40° C./75% RH.

TABLE 11a
6.0 g dose stored at 25° C./60% RH

| Stability time point (months) | T0 | T3 | T6 | T12 | T18 | T24 | T36 | T48 | T60 | T72 |
|---|---|---|---|---|---|---|---|---|---|---|
| pH after reconstitution: | | | | | | | | | | |
| ≤5.9 | 5.7 | 5.9 | 5.7 | 5.7 | 5.7 | 5.7 | 5.7 | 5.7 | 5.7 | 5.7 |
| Sodium oxybate HPLC Assay: | | | | | | | | | | |
| 5.40 to 6.60 g/unit | 5.96 | 5.97 | 5.96 | 6.02 | 5.99 | 6.22 | 6.21 | 6.09 | 6.02 | 5.97 |
| 90.0 to 110.0% of label claim | 99.3 | 99.5 | 99.3 | 100.3 | 99.8 | 103.7 | 103.5 | 101.6 | 100.3 | 99.6 |
| Related substances by HPLC (% | | | | | | | | | | |
| w/w): | | | | | | | | | | |
| Gamma-butyrolactone: ≤0.18% w/w | ≤0.05 | ≤0.05 | ≤0.05 | ≤0.05 | ≤0.05 | ≤0.05 | 0.10 | 0.08 | 0.10 | 0.10 |
| Individual unspecified impurity: | ND | ND | ND | ND | ND | ND | ND | ND | ND | ND |
| ≤0.10% w/w | | | | | | | | | | |
| Total related substances: ≤0.5% | ≤0.05 | ≤0.05 | ≤0.05 | ≤0.05 | ≤0.05 | ≤0.05 | 0.1 | 0.1 | 0.1 | 0.1 |
| w/w | | | | | | | | | | |
| Water content: | | | | | | | | | | |
| ≤4.0% w/w | 0.5 | 0.7 | 0.5 | 0.6 | 0.6 | 0.6 | 0.6 | 0.6 | 0.5 | 0.5 |
| 0.1N HCl Dissolution test by | | | | | | | | | | |
| HPLC: | | | | | | | | | | |
| (% dissolved) | | | | | | | | | | |
| 40% ≤ T1 hour ≤60% | 52 | 52 | 51 | 49 | 53 | 51 | 50 | 50 | 52 | 50 |
| 45% ≤ T3 hours ≤65% | | | | | | | 51 | 51 | 53 | 51 |
| 45% ≤ T4 hour ≤65% | 60 | 61 | 59 | 57 | 61 | 58 | 58 | 57 | 59 | 57 |
| T12 hours ≥ 80% | 101 | 100 | 99 | 96 | 102 | 98 | 98 | 98 | 102 | 100 |
| pH 6.8 dissolution test by HPLC: | | | | | | | | | | |
| (% dissolved) | | | | | | | | | | |
| T1 hour ≥ 85% | 98 | 97 | 100 | 100 | 99 | 102 | 100 | 99 | 100 | 96 |

ND: not detected
⁽⁴⁾ Modifications of specifications between T24 and T36 months

TABLE 11b
6.0 g dose stored at 30° C./65% RH

| Stability time point (months) | T0 | T6 | T24 |
|---|---|---|---|
| pH after reconstitution: | | | |
| ≤5.9⁽⁴⁾ | 5.7 | 5.7 | 5.7 |
| Sodium oxybate HPLC Assay: | | | |
| 5.40 to 6.60 g/unit | 5.96 | 6.02 | 6.02 |
| 90.0 to 110.0% of label claim | 99.3 | 100.3 | 100.3 |
| Related substances by HPLC (% w/w): | | | |
| Gamma-butyrolactone: ≤0.5% w/w | ≤0.05 | ≤0.05 | 0.06 |
| Individual unspecified impurity: ≤0.10% | ND | ND | ND |
| w/w | | | |
| Total related substances: ≤0.5% w/w | ≤0.05 | ≤0.05 | 0.1 |
| Water content: | | | |
| ≤4.0% w/w | 0.5 | 0.4 | 0.6 |
| 0.1N HCl Dissolution test by HPLC | | | |
| (% dissolved) | | | |
| 40% ≤ T1 hour ≤ 60% | 52 | 49 | 51 |
| 45% ≤ T4 hour ≤ 65% | 60 | 57 | 59 |
| T12 hours ≥ 80% | 101 | 99 | 98 |
| pH 6.8 dissolution test by HPLC (% | | | |
| dissolved) | | | |
| T1 hour ≥ 85% | 98 | 101 | 100 |

ND: not detected
⁽⁴⁾Modifications of specifications between T24 and T36 months

TABLE 11c
6.0 g dose stored at 40° C./75% RH

| Stability time point (months) | T0 | T3 | T6 |
|---|---|---|---|
| pH after reconstitution: | | | |
| ≤5.9⁽⁴⁾ | 5.7 | 5.9 | 5.8 |
| Sodium oxybate HPLC Assay: | | | |
| 5.40 to 6.60 g/unit | 5.96 | 6.25 | 2.95 |
| 90.0 to 110.0% of label claim | 99.3 | 104.2 | 99.2 |
| Related substances by HPLC (% w/w): | | | |
| Gamma-butyrolactone: ≤0.5% w/w | ≤0.05 | 0.08 | 0.14 |
| Individual unspecified impurity: ≤0.10% | ND | ND | ND |
| w/w | | | |
| Total related substances: ≤0.5% w/w | ≤0.05 | 0.10 | 0.10 |
| Water content: | | | |
| ≤4.0% w/w | 0.5 | 0.7 | 0.6 |
| 0.1N HCl Dissolution test by HPLC | | | |
| (% dissolved) | | | |
| 40% ≤ T1 hour ≤ 60% | 52 | 52 | 51 |
| 45% ≤ T4 hour ≤ 65% | 60 | 64 | 73* |
| T12 hours ≥ 80% | 101 | 100 | 100 |
| pH 6.8 dissolution test by HPLC (% | | | |
| dissolved) | | | |
| T1 hour ≥ 85% | 98 | 98 | 98 |

ND: not detected
*‘Out Of Specification’ value
⁽⁴⁾Modifications of specifications between T24 and T36 months

### Example 12: Storage Stability Testing of 7.5 g Batch of First Formulation According to Example 1

A study was performed to evaluate the stability of a batch of the first formulation manufactured as described in Example 1, at a dose of 7.5 g after storage for up to 72 months. The stability was measured after storage at a range of temperatures (25° C., 30° C., 40° C.) and at a range of relative humidities (RH) (60% RH, 65% RH, 75% RH).

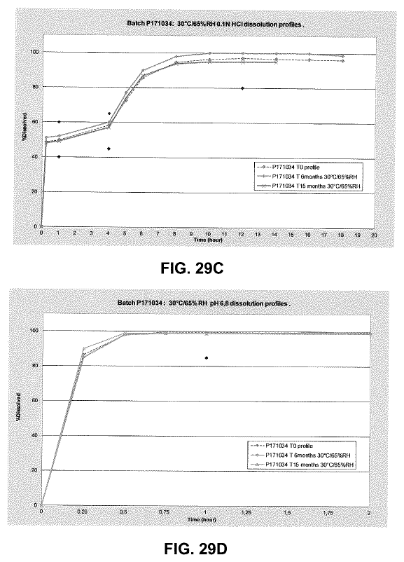

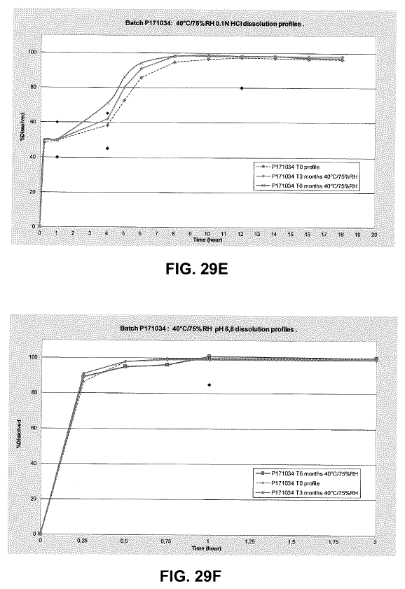

Tables 12a-12c include data for pH after reconstitution, amount of sodium oxybate by HPLC assay, related substances by HPLC (% w/w), water content, and dissolution of a batch of the 7.5 g dose of the formulation. FIGS. 29A-29F show results for a batch of the 7.5 g dose of the formulation. The data shown below in Tables 12a-12c and FIGS. 29A-29F show that the 7.5 g formulation is stable when stored up to at least 72 months at 25° C./60% RH, up to at least 15 months when stored at 30° C./65% RH, and up to at least 6 months at 40° C./75%.

TABLE 12a
7.5 g dose stored at 25° C./60% RH

| Stability time point (months) | T0 | T3 | T6 | T9 | T12 | T24 | T36 | T48 | T60 | T72 |
|---|---|---|---|---|---|---|---|---|---|---|
| pH after reconstitution: | | | | | | | | | | |
| ≤5.9⁽³⁾ | 5.7 | 5.7 | 5.7 | 5.7 | 5.7 | 5.7 | 5.7 | 5.7 | 5.7 | 5.7 |
| Sodium oxybate HPLC Assay: | | | | | | | | | | |
| 6.75 to 8.25 g/unit | 7.56 | 7.49 | 7.50 | 7.53 | 7.52 | 7.89 | 7.79 | 7.48 | 7.52 | 7.58 |
| 90.0 to 110.0% of label claim | 100.8 | 99.9 | 100.0 | 100.4 | 100.3 | 105.2 | 103.8 | 99.7 | 100.3 | 101.8 |
| Related substances by HPLC (% | | | | | | | | | | |
| w/w): | | | | | | | | | | |
| Gamma-butyrolactone: ≤0.18%⁽³⁾ w/w | ≤0.05 | ≤0.05 | ≤0.05 | ≤0.05 | ≤0.05 | ≤0.05 | 0.10 | 0.09 | 0.14 | 0.11 |
| Individual unspecified impurity: ≤0.10% | ND | ND | ND | ND | ND | ND | ND | ND | ND | ND |
| w/w | | | | | | | | | | |
| Total related substances: ≤0.5% w/w | ≤0.05 | ≤0.05 | ≤0.05 | ≤0.05 | ≤0.05 | ≤0.05 | 0.10 | 0.1 | 0.1 | 0.1 |
| Water content: | | | | | | | | | | |
| ≤4.0% w/w | 0.5 | 0.6 | 0.7 | 0.6 | 0.8 | 0.7 | 0.5 | 0.5 | 0.6 | 0.4 |
| 0.1N HCl Dissolution test by HPLC: | | | | | | | | | | |
| (% dissolved) | | | | | | | | | | |
| 40% ≤ T1 hour ≤60% | 50 | 51 | 50 | 51 | 51 | 51 | 50 | 48 | 50 | 51 |
| 45% ≤ T3 hours ≤65% | | | | | | | 51 | 49 | 51 | 52 |
| 45% ≤ T4 hour ≤65% | 58 | 58 | 57 | 59 | 59 | 59 | 57 | 55 | 58 | 59 |
| T12 hours ≥ 80% | 97 | 98 | 97 | 99 | 99 | 101 | 97 | 94 | 100 | 103 |
| pH 6.8 dissolution test by HPLC: | | | | | | | | | | |
| (% dissolved) | | | | | | | | | | |
| T1 hour ≥ 85% | 99 | 99 | 99 | 99 | 98 | 105 | 98 | 100 | 96 | 98 |

ND: not detected
⁽³⁾Modifications of specifications between T24 and T36 months

TABLE 12b
7.5 g dose stored at 30° C./65% RH

| Stability time point (months) | T0 | T6 | T15 |
|---|---|---|---|
| pH after reconstitution: | | | |
| ≤5.9⁽³⁾ | 5.7 | 5.7 | 5.7 |
| Sodium oxybate HPLC Assay: | | | |
| 6.75 to 8.25 g/unit | 7.56 | 7.46 | 7.78 |
| 90.0 to 110.0% of label claim | 100.8 | 99.5 | 103.7 |
| Related substances by HPLC (% w/w): | | | |
| Gamma-butyrolactone: ≤0.18% ⁽³⁾w/w | ≤0.05 | ≤0.05 | 0.06 |
| Individual unspecified impurity: ≤0.10% | ND | ND | ND |
| w/w | | | |
| Total related substances: ≤0.5% w/w | ≤0.05 | <0.05 | 0.1 |
| Water content: | | | |
| ≤4.0% w/w | 0.5 | 0.8 | 0.5 |
| 0.1N HCl Dissolution test by HPLC | | | |
| (% dissolved) | | | |
| 40% ≤ T1 hour ≤ 60% | 50 | 52 | 49 |
| 45% ≤ T4 hour ≤ 65% | 58 | 60 | 57 |
| T12 hours ≥ 80% | 97 | 100 | 95 |
| pH 6.8 dissolution test by HPLC (% | | | |
| dissolved) | | | |
| T1 hour ≥ 85% | 99 | 99 | 99 |

ND: not detected
⁽³⁾Modifications of specifications between T24 and T36 months

TABLE 12c
7.5 g dose stored at 40° C./75% RH

| Stability time point (months) | T0 | T3 | T6 |
|---|---|---|---|
| pH after reconstitution: | | | |
| ≤5.9⁽³⁾ | 5.7 | 5.7 | 5.7 |
| Sodium oxybate HPLC Assay: | | | |
| 6.75 to 8.25 g/unit | 7.56 | 7.41 | 7.43 |
| 90.0 to 110.0% of label claim | 100.8 | 98.8 | 99.1 |
| Related substances by HPLC (% w/w): | | | |
| Gamma-butyrolactone: ≤0.18% ⁽³⁾w/w | ≤0.05 | 0.07 | 0.13 |
| Individual unspecified impurity: ≤0.10% | ND | ND | ND |
| w/w | | | |
| Total related substances: ≤0.5% w/w | ≤0.05 | 0.10 | 0.10 |
| Water content: | | | |
| ≤4.0% w/w | 0.5 | 0.5 | 0.9 |
| 0.1N HCl Dissolution test by HPLC | | | |
| (% dissolved) | | | |
| 40% ≤ T1 hour ≤ 60% | 50 | 50 | 50 |
| 45% ≤ T4 hour ≤ 65% | 58 | 62 | 71* |
| T12 hours ≥ 80% | 97 | 98 | 98 |
| pH 6.8 dissolution test by HPLC (% | | | |
| dissolved) | | | |
| T1 hour ≥ 85% | 99 | 99 | 101 |

ND: not detected
*‘Out Of Specification’ value
⁽⁴⁾Modifications of specifications between T24 and T36 months

### Example 13: Storage Stability Testing of 9.0 g Batches of First Formulation According to Example 1

A study was performed to evaluate the stability of multiple batches of the first formulation manufactured as described in Example 1, at a dose of 9.0 g after storage for up to 72 months. The stability was measured after storage at a range of temperatures (25° C., 30° C., 40° C.) and at a range of relative humidities (RH) (60% RH, 65% RH, 75% RH).

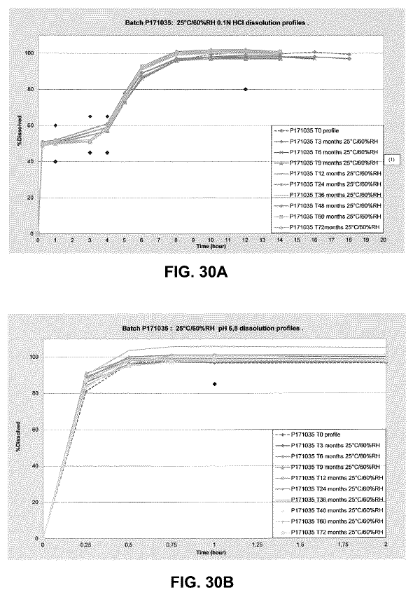

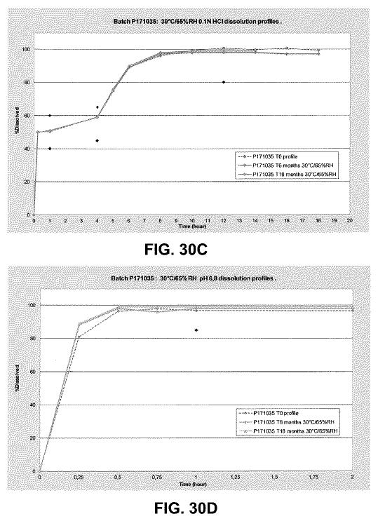

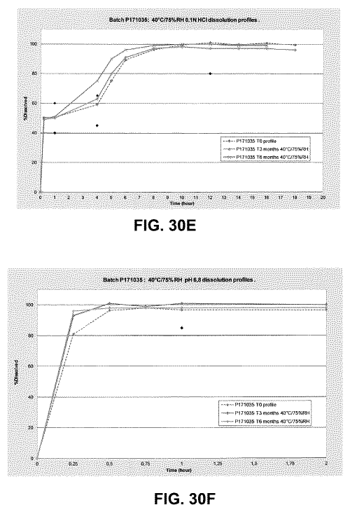

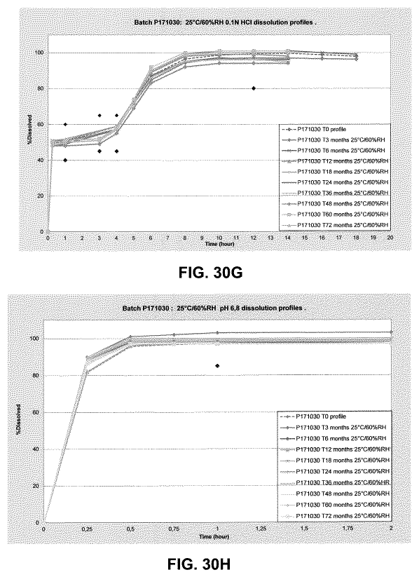

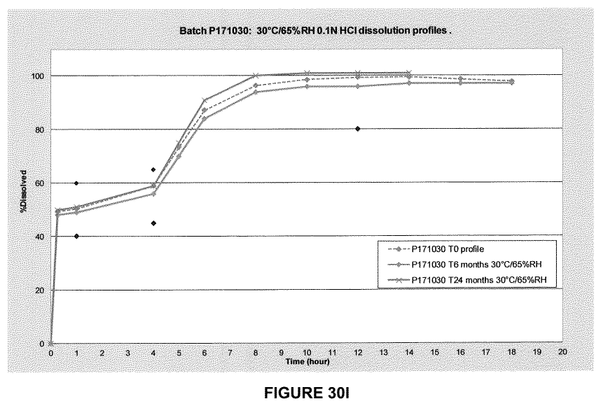

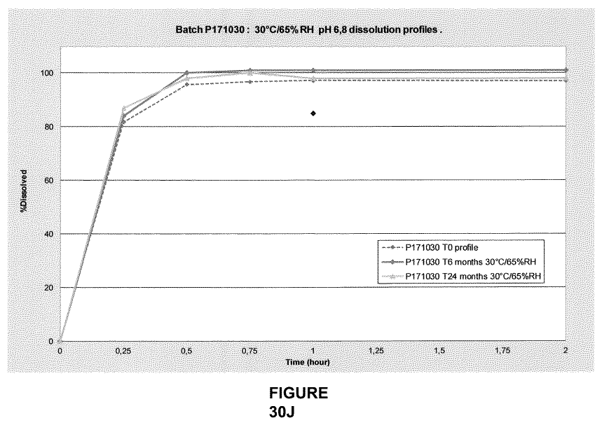

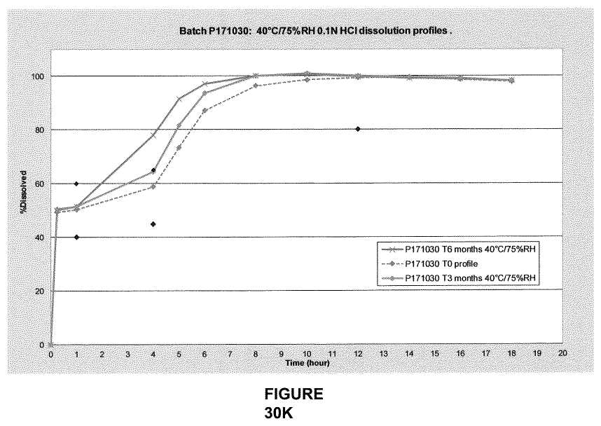

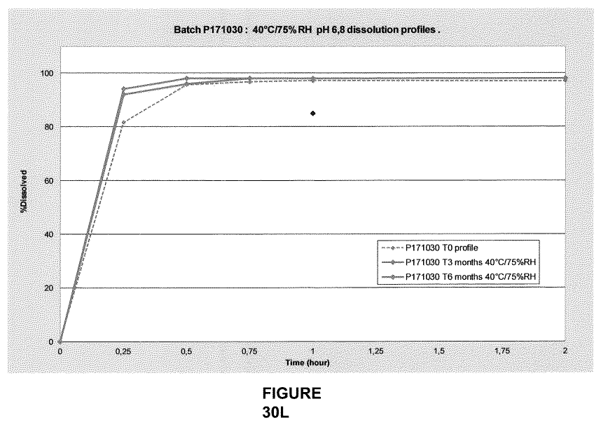

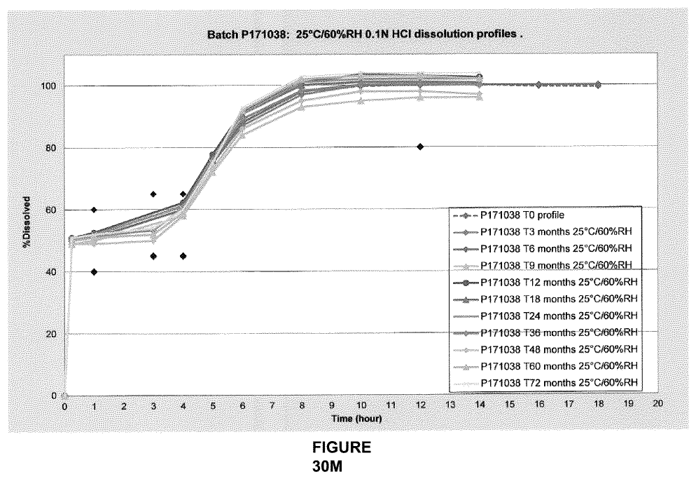

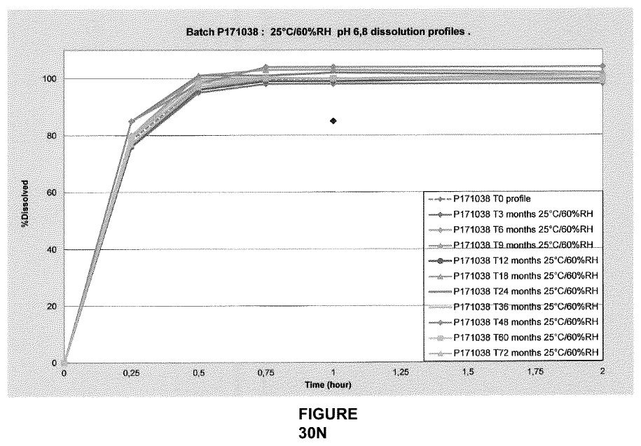

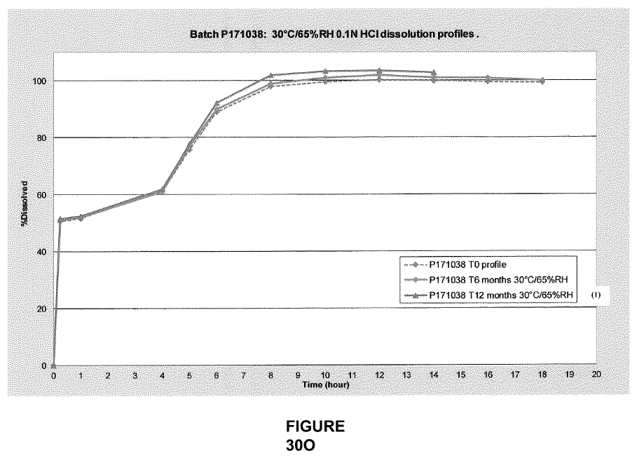

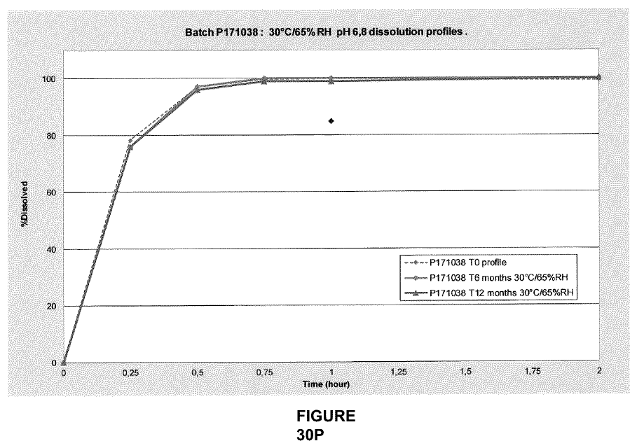

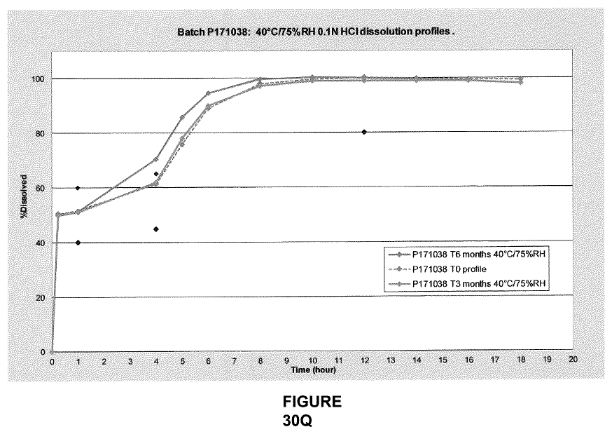

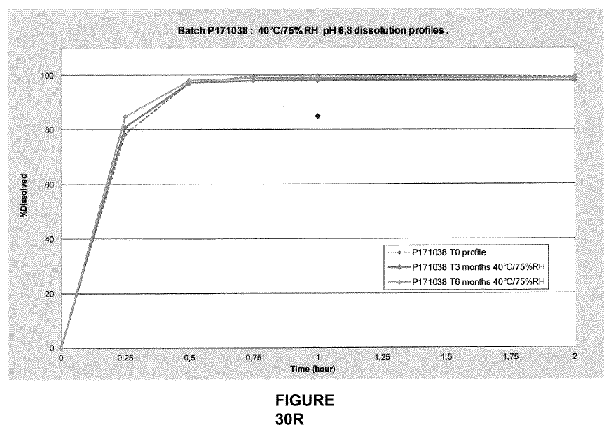

Tables 13a-13c include averages of pH after reconstitution, amount of sodium oxybate by HPLC assay, related substances by HPLC (% w/w), water content, and dissolution of multiple batches of the 9.0 g dose of the formulation. FIGS. 30A-30R show results for individual batches of the 9.0 g dose of the formulation. The data shown below in Tables 13a-13c and FIGS. 30A-30R show that the 9.0 g formulation is stable when stored up to at least 72 months at 25° C./60% RH, up to at least 24 months when stored at 30° C./65% RH, and up to at least 6 months at 40° C./75% RH.

TABLE 13a
9.0 g dose stored at 25° C./60% RH

| Stability time point (months) | T0 | T3 | T6 | T9 | T12 | T18 | T24 | T36 | T48 | T60 | T72 |
|---|---|---|---|---|---|---|---|---|---|---|---|
| pH after reconstitution: | | | | | | | | | | | |
| ≤5.9⁽³⁾ | 5.7 | 5.7 | 5.8 | 5.7 | 5.7 | 5.7 | 5.7 | 5.7 | 5.7 | 5.7 | 5.7 |
| Sodium oxybate HPLC Assay: | | | | | | | | | | | |
| 8.10 to 9.90 g/unit | 8.92 | 8.99 | 8.88 | 9.11 | 9.13 | 9.20 | 9.38 | 9.19 | 9.17 | 8.95 | 9.01 |
| 90.0 to 110.0% of label claim | 99.2 | 99.9 | 98.7 | 101.3 | 101.4 | 102.2 | 104.2 | 102.1 | 101.9 | 99.5 | 100.1 |
| Related substances by HPLC (% | | | | | | | | | | | |
| w/w): | | | | | | | | | | | |
| Gamma-butyrolactone: ≤0.18%⁽³⁾ | ≤0.05 | ≤0.05 | ≤0.05 | ≤0.05 | ≤0.05 | ≤0.05 | ≤0.05 | 0.08 | 0.08 | 0.11 | 0.11 |
| w/w | | | | | | | | | | | |
| Individual unspecified impurity: | ND | ND | ND | ND | ND | ND | ND | ND | ND | ND | ND |
| ≤0.10% w/w | | | | | | | | | | | |
| Total related substances: ≤0.5% | ≤0.05 | ≤0.05 | ≤0.05 | ≤0.05 | ≤0.05 | 0.06 | ≤0.05 | 0.10 | 0.1 | 0.1 | 0.1 |
| w/w | | | | | | | | | | | |
| Water content: | | | | | | | | | | | |
| ≤4.0% w/w | 0.73 | 0.63 | 0.67 | 0.65 | 0.87 | 0.55 | 0.43 | 0.63 | 0.53 | 0.63 | 0.60 |
| 0.1N HCl Dissolution test by | | | | | | | | | | | |
| HPLC: | | | | | | | | | | | |
| (% dissolved) | | | | | | | | | | | |
| 40% ≤ T1 hour ≤60% | 51 | 51 | 51 | 50 | 52 | 52 | 52.5 | 51 | 48 | 50 | 51 |
| 45% ≤ T3 hours ≤65% | | | | | | | | | 51 | 52 | 53 |
| 45% ≤ T4 hour ≤65% | 60 | 58 | 59 | 56 | 60 | 60 | 61 | 60 | 58 | 59 | 59 |
| T12 hours ≥ 80% | 98 | 100 | 99 | 97.5 | 100 | 100 | 84.3 | 100 | 82 | 101 | 85 |
| pH 6.8 dissolution test by HPLC: | | | | | | | | | | | |
| (% dissolved) | | | | | | | | | | | |
| T1 hour ≥ 85% | 100 | 98 | 100 | 99 | 99 | 103 | 100 | 102 | 100 | 98 | 98 |

ND: not detected
⁽³⁾Modifications of specifications

TABLE 13b
9.0 g dose stored at 30° C./65% RH

| Stability time point (months) | T0 | T6 | T12 | T18 | T24 |
|---|---|---|---|---|---|
| pH after reconstitution: | | | | | |
| ≤5.9⁽³⁾ | 5.7 | 5.8 | 5.7 | 5.7 | 5.7 |
| Sodium oxybate HPLC Assay: | | | | | |
| 8.10 to 9.90 g/unit | 8.97 | 9.10 | 9.00 | 9.16 | 9.05 |
| 90.0 to 110.0% of label claim | 99.6 | 101.1 | 100 | 101.8 | 100.5 |
| Related substances by HPLC (% w/w): | | | | | |
| Gamma-butyrolactone: ≤0.18% ⁽³⁾w/w | ≤0.05 | ≤0.05 | ≤0.05 | 0.08 | 0.11 |
| Individual unspecified impurity: ≤0.10% | ND | ND | ND | ND | ND |
| w/w | | | | | |
| Total related substances: ≤0.5% w/w | ≤0.05 | <0.05 | <0.05 | 0.1 | 0.1 |
| Water content: | | | | | |
| ≤4.0% w/w | 0.7 | 0.8 | 1.3 | 0.6 | 0.4 |
| 0.1N HCl Dissolution test by HPLC | | | | | |
| (% dissolved) | | | | | |
| 40% ≤ T1 hour ≤ 60% | 50 | 51 | 52 | 51 | 51 |
| 45% ≤ T4 hour ≤ 65% | 59 | 59 | 62 | 59 | 59 |
| T12 hours ≥ 80% | 99 | 99 | 104 | 99 | 101 |
| pH 6.8 dissolution test by HPLC (% | | | | | |
| dissolved) | | | | | |
| T1 hour ≥ 85% | 99 | 100 | 99 | 99 | 98 |

ND: not detected
⁽³⁾Modifications of specifications

TABLE 13c
9.0 g dose stored at 40° C./75% RH

| Stability time point (months) | T0 | T3 | T6 |
|---|---|---|---|
| pH after reconstitution: | | | |
| ≤5.9⁽³⁾ | 5.7 | 5.7 | 5.8 |
| Sodium oxybate HPLC Assay: | | | |
| 8.10 to 9.90 g/unit | 8.92 | 8.99 | 9.02 |
| 90.0 to 110.0% of label claim | 99.2 | 99.3 | 100.2 |
| Related substances by HPLC (% w/w): | | | |
| Gamma-butyrolactone: ≤0.18% ⁽³⁾w/w | ≤0.05 | 0.08 | 0.12 |
| Individual unspecified impurity: ≤0.10% | ND | ND | ND |
| w/w | | | |
| Total related substances: ≤0.5% w/w | ≤0.05 | 0.10 | 0.10 |
| Water content: | | | |
| ≤4.0% w/w | 0.7 | 0.7 | 0.7 |
| 0.1N HCl Dissolution test by HPLC | | | |
| (% dissolved) | | | |
| 40% ≤ T1 hour ≤ 60% | 51 | 51 | 51 |
| 45% ≤ T4 hour ≤ 65% | 60 | 63 | 74 |
| T12 hours ≥ 80% | 100 | 99 | 100 |
| pH 6.8 dissolution test by HPLC (% | | | |
| dissolved) | | | |
| T1 hour ≥ 85% | 98 | 99 | 98 |

ND: not detected
⁽³⁾Modifications of specifications between T24 and T36 months

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A pharmaceutical formulation stored within a package, the pharmaceutical formulation comprising: an immediate release portion comprising gamma-hydroxybutyrate or a pharmaceutically acceptable salt thereof; and a modified release portion comprising gamma-hydroxybutyrate or a pharmaceutically acceptable salt thereof, wherein the pharmaceutical formulation has a pH of less than 5.9 after the pharmaceutical formulation has been stored for up to at least 72 months at 25° C. to 40° C. and 60% to 75% relative humidity.

2. The pharmaceutical formulation of claim 1, wherein the pharmaceutical formulation has a pH of 5.6 to 5.9 after the pharmaceutical formulation has been stored for up to 72 months at 25° C. to 40° C. and 60% to 75% relative humidity.

3. The pharmaceutical formulation of claim 1, wherein the pharmaceutical formulation has a pH of 5.6 to 5.9 after the pharmaceutical formulation has been stored for up to 60 months at 25° C. to 40° C. and 60% to 75% relative humidity.

4. The pharmaceutical formulation of claim 1, wherein the pharmaceutical formulation has a pH of 5.6 to 5.9 after the pharmaceutical formulation has been stored for up to 48 months at 25° C. to 40° C. and 60% to 75% relative humidity.

5. The pharmaceutical formulation of claim 1, wherein the pharmaceutical formulation has a pH of 5.6 to 5.9 after the pharmaceutical formulation has been stored for up to 36 months at 25° C. to 40° C. and 60% to 75% relative humidity.

6. The pharmaceutical formulation of claim 1, wherein the pharmaceutical formulation has a pH of 5.6 to 5.9 after the pharmaceutical formulation has been stored for up to 24 months at 25° C. to 40° C. and 60% to 75% relative humidity.

7. The pharmaceutical formulation of claim 1, wherein the pharmaceutical formulation has a pH of 5.6 to 5.9 after the pharmaceutical formulation has been stored for up to 18 months at 25° C. to 40° C. and 60% to 75% relative humidity.

8. The pharmaceutical formulation of claim 1, wherein the pharmaceutical formulation has a pH of 5.6 to 5.9 after the pharmaceutical formulation has been stored for up to 12 months at 25° C. to 40° C. and 60% to 75% relative humidity.

9. The pharmaceutical formulation of claim 1, wherein the pharmaceutical formulation has a pH of 5.6 to 5.9 after the pharmaceutical formulation has been stored for up to 6 months at 25° C. to 40° C. and 60% to 75% relative humidity.

10. The pharmaceutical formulation of claim 1, wherein the pharmaceutical formulation has a pH of 5.6 to 5.9 after the pharmaceutical formulation has been stored for up to 3 months at 25° C. to 40° C. and 60% to 75% relative humidity.

11. The pharmaceutical formulation of claim 1, further comprising:
one or more suspending or viscosifying agents selected from the group consisting of xanthan gum, medium viscosity sodium carboxymethyl cellulose, mixtures of microcrystalline cellulose and sodium carboxymethyl cellulose, mixtures of microcrystalline cellulose and guar gum, medium viscosity hydroxyethyl cellulose, agar, sodium alginate, mixtures of sodium alginate and calcium alginate, gellan gum, carrageenan gum, medium viscosity hydroxypropylmethyl cellulose, and mixtures thereof; and
an acidifying agent selected from the group consisting of malic acid, citric acid, tartaric acid, adipic acid, boric acid, maleic acid, phosphoric acid, ascorbic acid, oleic acid, capric acid, caprylic acid, and benzoic acid,
wherein the suspending or viscosifying agent and the acidifying agent are separate and distinct from the immediate release portion and the modified release portion.

12. The pharmaceutical formulation of claim 11, wherein the suspending or viscosifying agent is present at 1% to 15% by weight of the formulation, and the acidifying agent is present at 1.2% to 15% by weight of the formulation.

13. The pharmaceutical formulation of claim 1, wherein the amount of gamma-hydroxybutyrate is equivalent to 4.5 g, 6.0 g, 7.5 g, or 9.0 g of sodium oxybate.

14. A pharmaceutical formulation stored within a package, the pharmaceutical formulation comprising: an immediate release portion comprising gamma-hydroxybutyrate or a pharmaceutically acceptable salt thereof; and a modified release portion comprising gamma-hydroxybutyrate or a pharmaceutically acceptable salt thereof, wherein less than 0.18% w/w gamma-butyrolactone is present in the pharmaceutical formulation after the pharmaceutical formulation has been stored for up to at least 72 months at 25° C. to 40° C. and 60% to 75% relative humidity.

15. The pharmaceutical formulation of claim 14, wherein less than 0.15% w/w gamma-butyrolactone is present in the pharmaceutical formulation after the pharmaceutical formulation has been stored for up to 72 months at 25° C. to 40° C. and 60% to 75% relative humidity.

16. The pharmaceutical formulation of claim 14, wherein less than 0.15% w/w gamma-butyrolactone is present in the pharmaceutical formulation after the pharmaceutical formulation has been stored for up to 60 months at 25° C. to 40° C. and 60% to 75% relative humidity.

17. The pharmaceutical formulation of claim 14, wherein less than 0.15% w/w gamma-butyrolactone is present in the pharmaceutical formulation after the pharmaceutical formulation has been stored for up to 48 months at 25° C. to 40° C. and 60% to 75% relative humidity.

18. The pharmaceutical formulation of claim 14, wherein less than 0.15% w/w gamma-butyrolactone is present in the pharmaceutical formulation after the pharmaceutical formulation has been stored for up to 36 months at 25° C. to 40° C. and 60% to 75% relative humidity.

19. The pharmaceutical formulation of claim 14, wherein less than 0.15% w/w gamma-butyrolactone is present in the pharmaceutical formulation after the pharmaceutical formulation has been stored for up to 24 months at 25° C. to 40° C. and 60% to 75% relative humidity.

20. The pharmaceutical formulation of claim 14, wherein less than 0.15% w/w gamma-butyrolactone is present in the pharmaceutical formulation after the pharmaceutical formulation has been stored for up to 18 months at 25° C. to 40° C. and 60% to 75% relative humidity.

21. The pharmaceutical formulation of claim 14, wherein less than 0.15% w/w gamma-butyrolactone is present in the pharmaceutical formulation after the pharmaceutical formulation has been stored for up to 12 months at 25° C. to 40° C. and 60% to 75% relative humidity.

22. The pharmaceutical formulation of claim 14, wherein less than 0.15% w/w gamma-butyrolactone is present in the pharmaceutical formulation after the pharmaceutical formulation has been stored for up to 6 months at 25° C. to 40° C. and 60% to 75% relative humidity.

23. The pharmaceutical formulation of claim 14, wherein less than 0.15% w/w gamma-butyrolactone is present in the pharmaceutical formulation after the pharmaceutical formulation has been stored for up to 3 months at 25° C. to 40° C. and 60% to 75% relative humidity.

24. The pharmaceutical formulation of claim 14, further comprising:
one or more suspending or viscosifying agents selected from the group consisting of xanthan gum, medium viscosity sodium carboxymethyl cellulose, mixtures of microcrystalline cellulose and sodium carboxymethyl cellulose, mixtures of microcrystalline cellulose and guar gum, medium viscosity hydroxyethyl cellulose, agar, sodium alginate, mixtures of sodium alginate and calcium alginate, gellan gum, carrageenan gum, medium viscosity hydroxypropylmethyl cellulose, and mixtures thereof; and
an acidifying agent selected from the group consisting of malic acid, citric acid, tartaric acid, adipic acid, boric acid, maleic acid, phosphoric acid, ascorbic acid, oleic acid, capric acid, caprylic acid, and benzoic acid,
wherein the suspending or viscosifying agent and the acidifying agent are separate and distinct from the immediate release portion and the modified release portion.

25. The pharmaceutical formulation of claim 24, wherein the suspending or viscosifying agent is present at 1% to 15% by weight of the formulation, and the acidifying agent is present at 1.2% to 15% by weight of the formulation.

26. The pharmaceutical formulation of claim 14, wherein the amount of gamma-hydroxybutyrate is equivalent to 4.5 g, 6.0 g, 7.5 g, or 9.0 g of sodium oxybate.

27. A pharmaceutical formulation stored within a package, the pharmaceutical formulation comprising: an immediate release portion comprising gamma-hydroxybutyrate or a pharmaceutically acceptable salt thereof; and a modified release portion comprising gamma-hydroxybutyrate or a pharmaceutically acceptable salt thereof, wherein the amount of gamma-hydroxybutyrate in the pharmaceutical formulation after the pharmaceutical formulation has been stored for up to at least 72 months at 25° C. to 40° C. and 60% to 75% relative humidity is 90.0% to 110.0% of the amount of gamma-hydroxybutyrate in the pharmaceutical formulation before storage.

28. The pharmaceutical formulation of claim 27, wherein the amount of gamma-hydroxybutyrate in the pharmaceutical formulation after the pharmaceutical formulation has been stored for up to 72 months at 25° C. to 40° C. and 60% to 75% relative humidity is 99.0% to 105.0% of the amount of gamma-hydroxybutyrate in the pharmaceutical formulation before storage.

29. The pharmaceutical formulation of claim 27, wherein the amount of gamma-hydroxybutyrate in the pharmaceutical formulation after the pharmaceutical formulation has been stored for up to 60 months at 25° C. to 40° C. and 60% to 75% relative humidity is 99.0% to 105.0% of the amount of gamma-hydroxybutyrate in the pharmaceutical formulation before storage.

30. The pharmaceutical formulation of claim 27, wherein the amount of gamma-hydroxybutyrate in the pharmaceutical formulation after the pharmaceutical formulation has been stored for up to 48 months at 25° C. to 40° C. and 60% to 75% relative humidity is 99.0% to 105.0% of the amount of gamma-hydroxybutyrate in the pharmaceutical formulation before storage.

31. The pharmaceutical formulation of claim 27, wherein the amount of gamma-hydroxybutyrate in the pharmaceutical formulation after the pharmaceutical formulation has been stored for up to 36 months at 25° C. to 40° C. and 60% to 75% relative humidity is 99.0% to 105.0% of the amount of gamma-hydroxybutyrate in the pharmaceutical formulation before storage.

32. The pharmaceutical formulation of claim 27, wherein the amount of gamma-hydroxybutyrate in the pharmaceutical formulation after the pharmaceutical formulation has been stored for up to 24 months at 25° C. to 40° C. and 60% to 75% relative humidity is 99.0% to 105.0% of the amount of gamma-hydroxybutyrate in the pharmaceutical formulation before storage.

33. The pharmaceutical formulation of claim 27, wherein the amount of gamma-hydroxybutyrate in the pharmaceutical formulation after the pharmaceutical formulation has been stored for up to 18 months at 25° C. to 40° C. and 60% to 75% relative humidity is 99.0% to 105.0% of the amount of gamma-hydroxybutyrate in the pharmaceutical formulation before storage.

34. The pharmaceutical formulation of claim 27, wherein the amount of gamma-hydroxybutyrate in the pharmaceutical formulation after the pharmaceutical formulation has been stored for up to 12 months at 25° C. to 40° C. and 60% to 75% relative humidity is 99.0% to 105.0% of the amount of gamma-hydroxybutyrate in the pharmaceutical formulation before storage.

35. The pharmaceutical formulation of claim 27, wherein the amount of gamma-hydroxybutyrate in the pharmaceutical formulation after the pharmaceutical formulation has been stored for up to 6 months at 25° C. to 40° C. and 60% to 75% relative humidity is 99.0% to 105.0% of the amount of gamma-hydroxybutyrate in the pharmaceutical formulation before storage.

36. The pharmaceutical formulation of claim 27, wherein the amount of gamma-hydroxybutyrate in the pharmaceutical formulation after the pharmaceutical formulation has been stored for up to 3 months at 25° C. to 40° C. and 60% to 75% relative humidity is 99.0% to 105.0% of the amount of gamma-hydroxybutyrate in the pharmaceutical formulation before storage.

37. The pharmaceutical formulation of claim 27, further comprising:
one or more suspending or viscosifying agents selected from the group consisting of xanthan gum, medium viscosity sodium carboxymethyl cellulose, mixtures of microcrystalline cellulose and sodium carboxymethyl cellulose, mixtures of microcrystalline cellulose and guar gum, medium viscosity hydroxyethyl cellulose, agar, sodium alginate, mixtures of sodium alginate and calcium alginate, gellan gum, carrageenan gum, medium viscosity hydroxypropylmethyl cellulose, and mixtures thereof; and
an acidifying agent selected from the group consisting of malic acid, citric acid, tartaric acid, adipic acid, boric acid, maleic acid, phosphoric acid, ascorbic acid, oleic acid, capric acid, caprylic acid, and benzoic acid,
wherein the suspending or viscosifying agent and the acidifying agent are separate and distinct from the immediate release portion and the modified release portion.

38. The pharmaceutical formulation of claim 37, wherein the suspending or viscosifying agent is present at 1% to 15% by weight of the formulation, and the acidifying agent is present at 1.2% to 15% by weight of the formulation.

39. The pharmaceutical formulation of claim 27, wherein the amount of gamma-hydroxybutyrate is equivalent to 4.5 g, 6.0 g, 7.5 g, or 9.0 g of sodium oxybate.

40. A pharmaceutical formulation stored within a package, the pharmaceutical formulation comprising: an immediate release portion comprising gamma-hydroxybutyrate or a pharmaceutically acceptable salt thereof; and a modified release portion comprising gamma-hydroxybutyrate or a pharmaceutically acceptable salt thereof, wherein the water content in the pharmaceutical formulation is ≤4.0% w/w after the pharmaceutical formulation has been stored for up to at least 72 months at 25° C. to 40° C. and 60% to 75% relative humidity.

41. The pharmaceutical formulation of claim 40, wherein the water content in the pharmaceutical formulation is ≤1.0% w/w after the pharmaceutical formulation has been stored for up to 72 months at 25° C. to 40° C. and 60% to 75% relative humidity.

42. The pharmaceutical formulation of claim 40, wherein the water content in the pharmaceutical formulation is ≤1.0% w/w after the pharmaceutical formulation has been stored for up to 60 months at 25° C. to 40° C. and 60% to 75% relative humidity.

43. The pharmaceutical formulation of claim 40, wherein the water content in the pharmaceutical formulation is ≤1.0% w/w after the pharmaceutical formulation has been stored for up to 48 months at 25° C. to 40° C. and 60% to 75% relative humidity.

44. The pharmaceutical formulation of claim 40, wherein the water content in the pharmaceutical formulation is ≤1.0% w/w after the pharmaceutical formulation has been stored for up to 36 months at 25° C. to 40° C. and 60% to 75% relative humidity.

45. The pharmaceutical formulation of claim 40, wherein the water content in the pharmaceutical formulation is ≤1.0% w/w after the pharmaceutical formulation has been stored for up to 24 months at 25° C. to 40° C. and 60% to 75% relative humidity.

46. The pharmaceutical formulation of claim 40, wherein the water content in the pharmaceutical formulation is ≤1.0% w/w after the pharmaceutical formulation has been stored for up to 18 months at 25° C. to 40° C. and 60% to 75% relative humidity.

47. The pharmaceutical formulation of claim 40, wherein the water content in the pharmaceutical formulation is ≤1.0% w/w after the pharmaceutical formulation has been stored for up to 12 months at 25° C. to 40° C. and 60% to 75% relative humidity.

48. The pharmaceutical formulation of claim 40, wherein the water content in the pharmaceutical formulation is ≤1.0% w/w after the pharmaceutical formulation has been stored for up to 6 months at 25° C. to 40° C. and 60% to 75% relative humidity.

49. The pharmaceutical formulation of claim 40, wherein the water content in the pharmaceutical formulation is ≤1.0% w/w after the pharmaceutical formulation has been stored for up to 3 months at 25° C. to 40° C. and 60% to 75% relative humidity.

50. The pharmaceutical formulation of claim 40, further comprising:
one or more suspending or viscosifying agents selected from the group consisting of xanthan gum, medium viscosity sodium carboxymethyl cellulose, mixtures of microcrystalline cellulose and sodium carboxymethyl cellulose, mixtures of microcrystalline cellulose and guar gum, medium viscosity hydroxyethyl cellulose, agar, sodium alginate, mixtures of sodium alginate and calcium alginate, gellan gum, carrageenan gum, medium viscosity hydroxypropylmethyl cellulose, and mixtures thereof; and
an acidifying agent selected from the group consisting of malic acid, citric acid, tartaric acid, adipic acid, boric acid, maleic acid, phosphoric acid, ascorbic acid, oleic acid, capric acid, caprylic acid, and benzoic acid,
wherein the suspending or viscosifying agent and the acidifying agent are separate and distinct from the immediate release portion and the modified release portion.

51. The pharmaceutical formulation of claim 50, wherein the suspending or viscosifying agent is present at 1% to 15% by weight of the formulation, and the acidifying agent is present at 1.2% to 15% by weight of the formulation.

52. The pharmaceutical formulation of claim 40, wherein the amount of gamma-hydroxybutyrate is equivalent to 4.5 g, 6.0 g, 7.5 g, or 9.0 g of sodium oxybate.

53. A pharmaceutical formulation stored within a package, the pharmaceutical formulation comprising: an immediate release portion comprising gamma-hydroxybutyrate or a pharmaceutically acceptable salt thereof; and a modified release portion comprising gamma-hydroxybutyrate or a pharmaceutically acceptable salt thereof, wherein after storage for up to at least 72 months at 25° C. to 40° C. and 60% to 75% RH, the pharmaceutical formulation has a dissolution of gamma-hydroxybutyrate that differs by less than 10% than the dissolution of gamma-hydroxybutyrate before the storage period when tested for at least three time points using a 0.1 N HCl dissolution test by HPLC or when tested after one hour using a pH 6.8 dissolution test by HPLC.

54. A pharmaceutical formulation stored within a package, the pharmaceutical formulation comprising: an immediate release portion comprising gamma-hydroxybutyrate or a pharmaceutically acceptable salt thereof; and a modified release portion comprising gamma-hydroxybutyrate or a pharmaceutically acceptable salt thereof, wherein after storage for up to at least 72 months at 25° C. to 40° C. and 60% to 75% RH, the pharmaceutical formulation has a dissolution of gamma-hydroxybutyrate of between about 40% and about 60% after 1 hour of dissolution in 0.1 N HCl, between about 45% and about 65% after 4 hours of dissolution in 0.1 N HCl, and greater than or equal to 80% dissolution after 12 hours of dissolution in 0.1 N HCl.

55. A pharmaceutical formulation stored within a package, the pharmaceutical formulation comprising: an immediate release portion comprising gamma-hydroxybutyrate or a pharmaceutically acceptable salt thereof; and a modified release portion comprising gamma-hydroxybutyrate or a pharmaceutically acceptable salt thereof, wherein after storage for up to at least 72 months at 25° C. to 40° C. and 60% to 75%, the pharmaceutical formulation has a dissolution of gamma-hydroxybutyrate of ≥85% after 1 hour in a pH 6.8 dissolution test by HPLC.
